SEQUENCE LISTING (80 sequences)

SEQ ID NO: 1 - protein, 17 aa, Homo sapiens
KKERKRLKEEKQKRKKE

SEQ ID NO: 2 - protein, 39 aa, Homo sapiens
KKERKRLKEEKQKRKKEARAAALAAAVAQDPAASGAPSS

SEQ ID NO: 3 - protein, 52 aa, Homo sapiens
PRSARFQELLQDLEKKERKLKEEKQKRKKEARAAALAAAVAQDPAASGAPSS

SEQ ID NO: 4 - protein, 222 aa, Homo sapiens
MAASVRQARSLLGVAATLAPGSRGYRARPPPRRRPGPRWPDPEDLLTPRWQLGPRYAAKQFARYGAASGVVPGSLWPSPEQLRELEAEEREWYPSLATMQESLRVKQLAEEQKRREREQHIAECMAKMPQMIVNWQQQQRENWEKAQADKERRARLQAEAQELLGYQVDPRSARFQELLQDLEKKERKRLKEEKQKRKKEARAAALAAAVAQDPAASGAPSS

SEQ ID NO: 5 - protein, 6 aa, Simian virus 40
KKKRKV

SEQ ID NO: 6 - protein, 17 aa, Homo sapiens
KRPAATKKAGQAKKKKL

SEQ ID NO: 7 - protein, 148 aa, Homo sapiens
EFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNSSPAPPQPAPGKVESGAGDAIGLADITQQLNQSELAVLLNLLQSQTDLSIP

SEQ ID NO: 8 - protein, 7 aa, Homo sapiens
YSPTSPS

SEQ ID NO: 9 - protein, 23 aa, Homo sapiens
EKKERKRLKEEKQKRKKEARAAA

SEQ ID NO: 10 - protein, 23 aa, Saccharomyces cerevisiae
ILLHRMEADRELHKRSKETSWYI

SEQ ID NO: 11 - protein, 16 aa, Homo sapiens
MECRNSPVTKTPPRDL

SEQ ID NO: 12 - protein, 4 aa, Artificial Sequence
  CDK consensus
XPXX

SEQ ID NO: 13 - DNA, 22 bp, Artificial Sequence
  Primer
tgtcatcaatggaaatcccatc

SEQ ID NO: 14 - DNA, 22 bp, Artificial Sequence
  Primer
taccgacctccagaactactgc

SEQ ID NO: 15 - DNA, 21 bp, Artificial Sequence
  Primer
tctcaaagctccgaaccatcc

SEQ ID NO: 16 - DNA, 24 bp, Artificial Sequence
  Primer
gaactttcaagaacactcagctcc

SEQ ID NO: 17 - DNA, 22 bp, Artificial Sequence
  Primer
ggatgccactgcttgttatgac

SEQ ID NO: 18 - DNA, 22 bp, Artificial Sequence
  Primer
tctggaatcagcctcttctctg

SEQ ID NO: 19 - DNA, 21 bp, Artificial Sequence
  Primer
gtgctcactcggttaaagcac

SEQ ID NO: 20 - DNA, 22 bp, Artificial Sequence
  Primer
gctaaaggaaacagggcatgtg

SEQ ID NO: 21 - DNA, 20 bp, Artificial Sequence
  Primer
gttcagcaagagaggcagac

SEQ ID NO: 22 - DNA, 22 bp, Artificial Sequence
  Primer
ttgcgtgtggagtatttggatg

SEQ ID NO: 23 - DNA, 20 bp, Artificial Sequence
  Primer
gtgaagaccatgacaggagg

SEQ ID NO: 24 - DNA, 21 bp, Artificial Sequence
  Primer
acttcaggtggtgtgtcagag

SEQ ID NO: 25 - DNA, 20 bp, Artificial Sequence
  Primer
agcgtggtatgagctactgc

SEQ ID NO: 26 - DNA, 20 bp, Artificial Sequence
  Primer
taagaagaccatcgcagagg

SEQ ID NO: 27 - DNA, 20 bp, Artificial Sequence
  Primer
cgaaggtggcaagaagaagc

SEQ ID NO: 28 - DNA, 22 bp, Artificial Sequence
  Primer
gcgctgcgtagaagtatcaatc

SEQ ID NO: 29 - DNA, 21 bp, Artificial Sequence
  Primer
cagaagtacaacgtgcgatcc

SEQ ID NO: 30 - DNA, 20 bp, Artificial Sequence
  Primer
ctacgcggctaagcagttcg

SEQ ID NO: 31 - DNA, 21 bp, Artificial Sequence
  Primer
taccaggcattgatctctcag

SEQ ID NO: 32 - DNA, 20 bp, Artificial Sequence
  Primer
acctcactgagcaccagaag

SEQ ID NO: 33 - DNA, 22 bp, Artificial Sequence
  Primer
gctgagaaactgatgaagctgc

SEQ ID NO: 34 - DNA, 20 bp, Artificial Sequence
  Primer
atcacgcgcttctgcaatcc

SEQ ID NO: 35 - DNA, 21 bp, Artificial Sequence
  Primer
ccagctaacagtaacaccaac

SEQ ID NO: 36 - DNA, 20 bp, Artificial Sequence
  Primer
tactggtgctcttgaaggac

SEQ ID NO: 37 - DNA, 20 bp, Artificial Sequence
  Primer
gccaaggaagctctggtctg

SEQ ID NO: 38 - DNA, 21 bp, Artificial Sequence
  Primer
gtcccactgacgagttctatg

SEQ ID NO: 39 - DNA, 21 bp, Artificial Sequence
  Primer
tacattggctgggataagcac

SEQ ID NO: 40 - DNA, 24 bp, Artificial Sequence
  Primer
ctggagaaagagaacgcctcaaag

SEQ ID NO: 41 - DNA, 22 bp, Artificial Sequence
  Primer
aaagttgtcatggatgaccttg

SEQ ID NO: 42 - DNA, 21 bp, Artificial Sequence
  Primer
taacatcaggccacacagctg

SEQ ID NO: 43 - DNA, 21 bp, Artificial Sequence
  Primer
caacggtggatgagtggtctc

SEQ ID NO: 44 - DNA, 22 bp, Artificial Sequence
  Primer
gctggcatccaacttcttgatc

SEQ ID NO: 45 - DNA, 21 bp, Artificial Sequence
  Primer
gctgcttccactctgtacgtg

SEQ ID NO: 46 - DNA, 23 bp, Artificial Sequence
  Primer
gtatcagtagtatgagcagcagc

SEQ ID NO: 47 - DNA, 21 bp, Artificial Sequence
  Primer
aggatgtcttgctgccatctg

SEQ ID NO: 48 - DNA, 22 bp, Artificial Sequence
  Primer
caagcacagcagtgagatgctc

SEQ ID NO: 49 - DNA, 22 bp, Artificial Sequence
  Primer
gcttctttcaggcgtttattcc

SEQ ID NO: 50 - DNA, 22 bp, Artificial Sequence
  Primer
agtgtgatgatggtgaggatgg

SEQ ID NO: 51 - DNA, 21 bp, Artificial Sequence
  Primer
tgtctccgcttggagtgtatc

SEQ ID NO: 52 - DNA, 22 bp, Artificial Sequence
  Primer
gctggtatcccataaggaaaga

SEQ ID NO: 53 - DNA, 20 bp, Artificial Sequence
  Primer
gcatccttagattcccaccg

SEQ ID NO: 54 - DNA, 24 bp, Artificial Sequence
  Primer
cctctgtattggacatcacgattc

SEQ ID NO: 55 - DNA, 23 bp, Artificial Sequence
  Primer
gatttcttaattccacctgtggc

SEQ ID NO: 56 - DNA, 20 bp, Artificial Sequence
  Primer
ctgaacccaacccaaacctc

SEQ ID NO: 57 - DNA, 20 bp, Artificial Sequence
  Primer
cctacgtggacagttgtgcc

SEQ ID NO: 58 - DNA, 20 bp, Artificial Sequence
  Primer
gtaccattcgcgttcttcgg

SEQ ID NO: 59 - DNA, 20 bp, Artificial Sequence
  Primer
ccacattctgcacatacgtg

SEQ ID NO: 60 - DNA, 20 bp, Artificial Sequence
  Primer
gaggtcttcgctggtagcaa

SEQ ID NO: 61 - DNA, 20 bp, Artificial Sequence
  Primer
ccagtttgtgcagttccagt

SEQ ID NO: 62 - DNA, 20 bp, Artificial Sequence
  Primer
ttgcttcctgggagaggtct

SEQ ID NO: 63 - DNA, 20 bp, Artificial Sequence
  Primer
ctcagggccaccaagttcat

SEQ ID NO: 64 - DNA, 20 bp, Artificial Sequence
  Primer
gattcctcagagtccaggcc

SEQ ID NO: 65 - DNA, 20 bp, Artificial Sequence
  Primer
catactccaccggactgcta

SEQ ID NO: 66 - DNA, 21 bp, Artificial Sequence
  Primer
catggcgtgcttctcctgaag

SEQ ID NO: 67 - DNA, 20 bp, Artificial Sequence
  Primer
caagtgctgacttcaaggtc

SEQ ID NO: 68 - DNA, 20 bp, Artificial Sequence
  Primer
ggagtgatttcagccaccaa

SEQ ID NO: 69 - DNA, 22 bp, Artificial Sequence
  Primer
tgtcatcaatggaaatcccatc

SEQ ID NO: 70 - DNA, 21 bp, Artificial Sequence
  Primer
aagaccgggctgaagtaaagc

SEQ ID NO: 71 - DNA, 22 bp, Artificial Sequence
  Primer
gtagatatgaagcgtgccgtag

SEQ ID NO: 72 - DNA, 20 bp, Artificial Sequence
  Primer
tgcaggctcaagcaatcctc

SEQ ID NO: 73 - DNA, 20 bp, Artificial Sequence
  Primer
agccactaggtgacactagc

SEQ ID NO: 74 - DNA, 20 bp, Artificial Sequence
  Primer
catgccctgctgtctacctg

SEQ ID NO: 75 - DNA, 20 bp, Artificial Sequence
  Primer
cacgcagtcaagtgtgtgtg

SEQ ID NO: 76 - DNA, 20 bp, Artificial Sequence
  Primer
aggcttcgaaagaactggcc

SEQ ID NO: 77 - DNA, 20 bp, Artificial Sequence
  Primer
gctctcctccactacagtac

SEQ ID NO: 78 - DNA, 20 bp, Artificial Sequence
  Primer
ctgggcaacatggtgagacc

SEQ ID NO: 79 - DNA, 20 bp, Artificial Sequence
  Primer
ctacacgctgcttccggttc

SEQ ID NO: 80 - DNA, 20 bp, Artificial Sequence
  Primer
gagtggcagacgattggtcg

(19) United States
(12) Patent Application Publication
(10) Pub. No.: US 2017/0157212 A1
(43) Pub. Date: Jun. 8, 2017

(54) CDK MODULATORS AND METHODS FOR THE TREATMENT OF CANCER

(71) Applicant: Salk Institute for Biological Studies, La Jolla, CA (US)

(72) Inventors: Katherine Jones, La Jolla, CA (US); Seung Hyuk Choi, La Jolla, CA (US)

(73) Assignee: Salk Institute for Biological Studies, La Jolla, CA (US)

(21) Appl. No.: 15/368,341

(22) Filed: Dec. 2, 2016

Related U.S. Application Data
(60) Provisional application No. 62/262,853, filed on Dec. 3, 2015.

(51) Int. Cl. A61K 38/17; A61K 45/06; C07K 14/47

(52) U.S. Cl. CPC A61K 38/1709; C07K 14/47; A61K 45/06; C12Y 207/11023; A61K 48/00

(57) ABSTRACT

Disclosed is a polypeptide that includes amino acids 183-222 of CRIF, wherein the polypeptide does not include the full length CRIF1 amino acid sequence. Also disclosed is a nucleic acid molecule encoding this polypeptide, vectors including this nucleic acid molecule, and host cells transformed with these vectors. In some embodiments, methods are disclosed for treating a subject with cancer, comprising administering to the subject a therapeutically effective amount of an inhibitor of CDK12/CRIF1 interaction, thereby treating the cancer in the subject. In specific non-limiting examples, these methods can utilize CRIF1 polypeptides, nucleic acids encoding these polypeptides, and vectors including these nucleic acids.

### PRIORITY CLAIM

This application claims the benefit of U.S. Provisional Application No. 62/262,853, filed Dec. 3, 2015, which is herein incorporated by reference in its entirety.

### ACKNOWLEDGMENT OF GOVERNMENT SUPPORT

This invention was made with government support under Grant Number 5 R01 CA 125535 awarded by NIH/NCI. The government has certain rights in the invention.

### FIELD

This application relates to the field of cancer, specifically to the use of agents, specifically Growth Arrest and DNA Damage-Inducible Proteins-Interacting Protein 1 (known as GADD45GIP1 or CRIF1) polypeptides, that inhibit the activity of cyclin dependent kinase (Cdk)12, and their use for the treatment of cancer.

### BACKGROUND

The C-terminal domain of human RNA polymerase II (RNAPII) contains 52 repeats of a consensus heptad (Y₁S₂P₃T₄S₅P₆S₇, SEQ ID NO: 8) that are differentially phosphorylated during the transcription cycle (Bowman and Kelly, Nucleus 5, 224-236, 2014; Jonkers and Lis, Nat Rev Mol Cell Biol 16, 167-177, 2015). Phosphorylation at the Ser2 position in the CTD in metazoans is predominantly mediated by CDK12:CCNK (Bartkowiak et al, Genes Dev 24, 2303-2316, 2010), which stimulates productive transcription elongation, cotranscriptional splicing, and mRNA 3′-end processing to control steady-state mRNA levels for a small set (2-3%) of protein coding genes (Bartkowiak and Greenleaf, J Biol Chem 290, 1786-1795, 2012; Blazek et al., Genes Dev 25, 2158-2172, 2011; Cheng et al., Mol Cell Biol 32, 4691-4704, 2012; Davidson et al., Genes Dev 28, 342-356, 2014; Eifler et al., Mol Cell Biol 35, 468-478, 2015; Liang et al., Mol Cell Biol 35, 928-938, 2015). In contrast, the closely-related positive elongation factor, P-TEFb (CDK9:CCNT1) controls early transcription elongation at a much larger set of target genes. RNAPII CTD-Ser2P most frequently peaks at the 3′ end of active genes, but is also found at the 5′ end of a subset of genes (Schwartz et al. Genes Dev 26, 2690-2695, 2012). Many of the CDK12 target genes are required for homologous recombination DNA repair, including BRCA1, ATR, FANC1, and FANCD2, and consequently CDK12 is important for genome stability (Blazek et al, Genes Dev 25, 2158-2172, 2011). In addition, CDK12 is required for the induction of c-FOS transcription in growth factor signaling cells (Eifler et al., Mol Cell Biol 35, 468-478, 2015) activation of NRF2-dependent genes by oxidative stress (Li et al., Sci Rep 6, 21455, 2016), and genes required for embryonic development (Juan et al., Cell Death Differ 23, 1038-1048, 2016) and self-renewal of human embryonic stem cells (Dai et al., J Biol Chem 287, 25344-25352, 2012).

Consistent with its role in genome stability, CDK12 is a tumor suppressor, and also enables cancer cells to resist chemotherapy-induced cell death (Chilà et al., Cancer Treat Rev 50, 83-88, 2016). Mutations that disrupt the stability or kinase activity of the CDK12 complex are commonly found in serous ovarian cancers (Ekumi et al., Nucleic Acids Res 43, 2575-2589, 2015), and CDK12 is often highly expressed in HER2-positive breast cancers, as part of the ERBB2/HER2 amplicon (Mertins et al., Nature 534, 55-62, 2016). Inhibition or loss of CDK12 kinase activity strongly sensitizes cancer cells to undergo apoptosis in response to PARP1/2 inhibitors or drugs that induce DNA damage (Bajrami et al., Cancer Res 74, 287-297., 2014; Blazek et al., Genes Dev 25, 2158-21722011). Loss of CDK12 results in defects in homologous recombination similar to those seen in cells lacking BRCA1, as well as other chromosomal alterations, such as massive tandem DNA duplications, which can further disrupt genome stability (Popova et al., Cancer Res 76, 1882-18912016). Thus, CDK12 is a target for the development of anti-cancer drugs. Screens for such compounds have identified redox-sensitive compounds that inhibit CDK12 and block tumor growth in pre-clinical studies (Chipumuro et al., Cell 159, 1126-1139, 2014; Christensen et al., Cancer Cell 26, 909-922, 2014; Wang et al., Cell 163, 174-186, 2015, Zhang et al., Nat Chem Biol 12, 876-884, 2016). However, a need remains for agents that inhibit the activity of CDK12, which can be used to prevent chemoresistance and treat cancer.

### SUMMARY

It is disclosed herein that CRIF1 polypeptides inhibit CDK12 kinase activity and/or nuclear localization, and that these CRIF1 polypeptides can be used to treat cancer. These polypeptides include the amino acid sequence set forth as SEQ ID NO: 1.

In some embodiments, a polypeptide is disclosed that includes, or consists of, the amino acid sequence set forth as SEQ ID NO::2. Also disclosed is a nucleic acid molecule encoding this polypeptides, vectors including this nucleic acid molecule, and host cells transformed with these vectors.

In some embodiments, methods are disclosed for treating a subject with cancer, comprising administering to the subject a therapeutically effective amount of an inhibitor of CDK12 kinase activity, such as a CRIF1 polypeptide (examples include, but are not limited to, SEQ ID NO: 1, 2 or 3), thereby treating the cancer in the subject. In specific non-limiting examples, these methods can utilize CRIF1 polypeptides, nucleic acids encoding these polypeptides, and vectors including these nucleic acids.

The foregoing and other objects and features of the disclosure will become more apparent from the following detailed description, which proceeds with reference to the accompanying figures.

### BRIEF DESCRIPTION OF THE DRAWINGS

FIGS. 1A-1G. CDK12 Selectively Regulates the Translation of CHK1 mRNA. (A) Analysis of the effects of CDK12 on CHK1 expression and activation of p53 in response to DNA damage. U2OS cells were transfected with a control siRNA, or siRNAs targeting CDK12 or CCNK, as indicated. After 48 hr, cells were either untreated or treated with Etoposide for 12 hr to monitor induction of p53. Expression of p53, total and activated (S317P) CHK1, and total HDM2, was monitored by immunoblot. Note that depletion of either CDK12 or CCNK was sufficient to destabilize both subunits of the CDK12 complex in vivo. (B) Schematic of the role of CHK1 in p53 induction, mediated through its effects on CHK1 expression. (C) Immunoblot as in (A), except that DNA damage was induced by Hydroxyurea (HU). (D) Transcription of CHK1 is unaffected by knockdown of CDK12. CHK1 mRNA levels were analyzed by qRT-PCR in U2OS cells depleted of CDK12 or CCNK, as described in (A), and normalized to GAPDH expression. (E) CHK1 protein stability is not affected in CDK12-depleted cells. U2OS cells were treated with cycloheximide to block de novo protein synthesis, and steady-state CHK1 protein levels were examined by immunoblot in cells transfected with control or CDK12-targeted siRNAs (FIG. 8D). The graph shows relative CHK1 protein levels at different timepoints in untreated or CDK12-depleted cells. (F) CDK12 is required for biosynthesis of CHK1 protein. U2OS cells treated with the indicated siRNAs were subjected to pulse-chase analysis with AHA-Met to measure de novo Chk1 protein production as measured by SDS-PAGE analysis of anti-CHK1 immunoprecipitates. (G) CDK12 is selectively required for the loading of CHK1 mRNA onto polysomes. U2OS cells were treated with control or CDK12-specific siRNAs, as indicated, and extracts were fractionated in sucrose gradients to monitor polyribosome-associated CHK1 and GAPDH mRNAs by qRT-PCR.

FIGS. 2A-2E. CDK12 Selectively Regulates the Association of eIF4G with CHK1 mRNA. (A) The left panel shows a schematic of the phosphorylation-mediated exchange of 4E-BP1 and eIF4G on target mRNAs required for translation initiation. Phosphorylation of 4E-BP1 is mediated by mTORC1 (T37, T46) together with a Ser-Pro kinase (S65, T70). RNA immunoprecipitation experiments shown on the right side monitor the binding of CHK1 and GAPDH mRNAs to eIF4G and eIF4E in cells treated with control or CDK12-specific siRNAs. Protein:mRNA complexes from U2OS cells were isolated by immunoprecipitation using the indicated antisera, and associated RNAs were measured by qRT-PCR. (B) List of the top 50 mRNAs that associate with eIF4G in a CDK12-dependent manner, as identified by RIP-seq analysis from (A). Changes of RNA abundance are shown in log 2 scale. The total RNA changes of the top 50 genes are also shown in the left lanes. (C) The mRNAs enriched in anti-eIF4G immunoprecipitates from U2OS cells treated with control or CDK12-specific (siCDK12) siRNAs, or with Rapamycin, were examined by RIP-seq. Following exclusion of the transcriptional targets of CDK12 (FIG. 9C), the overlap between primary translation targets of CDK12 and Rapamycin is summarized using a Venn diagram. (D) Summary of the extent to which the top-fifty mRNAs that depend on both CDK12 also require mTORC1 (Rapamycin-sensitive) for binding to eIF4G, and visa-versa. (E) Validation of the RIP-seq data. Shown are mRNAs that bind eIF4G in a manner that requires Cdk12 and mTORC1, individually or together. Target genes tested were selected from top-fifty mRNAs identified in the RIP-seq dataset (B).

FIGS. 3A-3D. CDK12, but not CDK9/P-TEFb, Phosphorylates 4E-BP1 at S65 and T70 In Vivo and In Vitro. (A) Global 4E-BP1 phosphorylation at S65 and T70 is lost in U2OS cells depleted of CDK12 or CCNK. Shown is an immunoblot using phospho-specific antisera to monitor 4E-BP1 phosphorylation at S65, T70, or T37/46. (B) 4E-BP1 is selectively phosphorylated by CDK12, but not by CDK9/P-TEFb, in vivo. Immunoblot analysis was as described in (A). (C) Affinity-purified Raptor (mTORC1) and CDK12 kinase complexes co-operate to phosphorylate 4E-BP1 in vitro. The Raptor (mTORC1) and CDK12 kinase complexes were incubated, alone and together, with purified recombinant 4E-BP1 in cell-free kinase assays and analyzed for site-specific phosphorylation as in (A). (D) Site-specific 4E-BP1 phosphorylation at T37 and T46 by mTORC1 primes CDK12 phosphorylation at S65 and T70 in vitro. Recombinant wild-type and point mutant 4E-BP1 proteins were incubated with affinity-purified Raptor (mTORC1) and CDK12 in vitro, as in (C). The schematic at the bottom illustrates how mTORC1 and CDK12 cooperate to phosphorylate independent sites in 4E-BP1. SEQ ID NO: 11 (4EBP1) and SEQ ID NO: 12 (CDK consensus) are shown.

FIGS. 4A-4B. CDK12 Promotes Cotranscriptional Loading of 4E-BP1 onto Nascent mRNAs at Target Gene Promoters In Vivo. (A) CDK12 regulates the release of 4E-BP1 at the CHK1 gene promoter. ChIP analysis of CDK12, RNAPII CTD-Ser2P, and 4E-BP1 and eIF4E at the CHK1 gene in U2OS cells treated with control (siCTL; black bars) or CDK12-specific (siCDK12; white bars) siRNAs. ChIP experiments used the specific antisera listed above each graph. The ratio of P-T70 of 4E-BP1/total 4E-BP1 is shown in the last panel. The bottom schematic indicates the genomic location of the CHK1 gene primers in this assay. The schematic at the right bottom illustrates the phosphorylation and release of 4E-BP1 from nascent mRNAs at CDK12 target genes. (B) Phosphorylation of 4E-BP1 at the CHK1 and other CDK12 target gene promoters (RPL26, TMA7) is sensitive to the mTORC1 inhibitor, Rapamycin. The ChIP experiment is as described in (A).

FIGS. 5A-5I. The Mitochondrial OX-PHOS Regulator, CRIF1, is a Novel Subunit of the Human CDK12 Kinase Complex. (A) Identification of CRIF1 as a near-stoichiometric subunit of the native CDK12 kinase complex. MudPIT analysis of CDK12 complexes from HEK293 cells that stably express HA-tagged full-length human CDK12. The HA-CDK12 complex was affinity-purified using anti-HA antibody and visualized by SDS-PAGE and silver-stain. (B) List of the identified proteins in the HA-CDK12 complex from (A). (C) Endogenous CRIF1 co-immunoprecipitates with native CDK12 in U2OS cell extracts. Shown is an immunoblot analysis of CRIF1, CCNK, and the chaperone proteins CDC37 and HSP90 in an anti-CDK12 immunoprecipitate. (D) CRIF1 is a specific subunit of the CDK12 complex. Native CRIF1 was immunoprecipitated from U2OS cells using an anti-CRIF1 antibody and analyzed by co-immunoprecipitation for association with various CDKs, as indicated. (E) CRIF1 binds to regulatory loop region in CDK12. HEK293 cells were co-transfected with HA-CDK12 fragments with full-length FLAG-CRIF1. Expressed HA-CDK12 fragments were co-immunoprecipitated and tested for binding to FLAG-CRIF1 by immunoblot using the indicated antibodies. The domain in CDK12 that binds to CRIF1 is illustrated at the bottom. (F) The C-terminal motif of CRIF1 mediates binding to CDK12. FLAG-CRIF1 proteins containing different C-terminal truncations were co-expressed with HA-CDK12 (aa 985-1490) and analyzed for binding by co-immunoprecipitation and immunoblot. The schematic at the bottom illustrates that CDK12 binds to a region overlapping the CRIF1 nuclear localization sequence (NLS). The bottom diagram shows that the amino acid sequence of this region of CRIF1 (SEQ ID NO: 9) is related to a domain in the yeast CTK3 protein (SEQ ID NO: 10). (G) CRIF1 is essential for cell survival in galactose media. U2OS cells were transfected with CDK12 or CRIF1-specific siRNAs, and grown in either glucose or galactose as sole energy source. Cell viability was analyzed after 72 hr. (H) CRIF1 inhibits global RNAPII CTD Ser2 phosphorylation levels. Immunoblot analysis of the effect of siRNAs targeted to CCNK, CDK12, or CRIF1 on total RNAPII or RNAPII CTD-Ser2P and CTD-Ser5P, as indicated, in U2OS cells. Expression of the mitochondrial MTCO-1 (COX1) protein was assessed using two different antisera. (I) Analysis of CRIF1 and CDK12 subcellular localization following fractionation of U2OS cells. U2OS cells were treated with control (CTL) or CRIF1-specific siRNA, and extracts were subjected to subcellular fractionation. Each fraction (T; total, C:cytosolic, M:membrane/organelle, and N:nuclear) was analyzed by immunoblot using the indicated antibodies.

FIGS. 6A-6H. CRIF1 Inhibits CDK12 Kinase Activity In Vivo and In Vitro.

(A) CRIF1 and CDK12 have opposite effects on global RNAPII CTD-Ser2P levels in vivo. U2OS cells were transfected with indicated siRNAs for 48 hr, and RNAPII phosphorylation was monitored by immunoblot using phospho-specific CTD (Ser2P and Ser5P) antibodies. (B) Knockdown of CRIF1 up-regulates CHK1 expression and induction of p53. U2OS cells were transfected with control (CTL) or CRIF1-specific siRNAs, in the presence or absence of etoposide to induce DNA damage, and analyzed by immunoblot using the indicated antisera. (C) Analysis of steady-state mRNA levels by qRT-PCR in extracts from U2OS cells exposed to control (CTL) or CRIF1-specific siRNAs, as indicated. (D) Depletion of CRIF1 enhances 4E-BP1 phosphorylation at S65 and T70 in vivo, as assessed by immunoblot using phospho-specific antibodies. (E) CRIF1 inhibits RNAPII CTD-Ser2 phosphorylation by CDK12 complexes in vitro. The human GST-CTD (52 repeat) protein was incubated in vitro with the affinity-purified HA-CDK12 kinase complex in the absence or presence of increasing levels of purified recombinant CRIF1, and analyzed by immunoblot with the indicated antisera. (F) Recombinant CRIF1 does not inhibit the activity of the CDK9/P-TEFb kinase in vitro. Analysis of GST-CTD Ser2 phosphorylation was carried out as described in (E), using the affinity-purified FLAG-CDK9 kinase. (G) CRIF1 inhibits 4E-BP1 phosphorylation by CDK12 in vitro. GST-CTD Ser2 phosphorylation was monitored by immunoblot in the absence or in the presence of different levels of affinity-purified recombinant CRIF1. (H) Depletion of CRIF1 selectively enhances the association of eIF4G with CDK12 target mRNAs (CHK1, RPL26, TWAT), but not GAPDH mRNA. RNA immunoprecipitation and qRT-PCR measurements were carried out in U2OS cells transfected with control (CTL) or CRIF1-specific siRNAs. RNA immunoprecipitation with IgG was performed in parallel as control. The bottom panel shows that knockdown of CRIF1 does not affect total mRNA levels of these transcripts as measured by qRT-PCR.

FIGS. 7A-7F. Oxidative Stress Blocks the Binding of CRIF1 to CDK12 and Up-Regulates DNA Damage and Cell Stress Survival. (A) PARP activity is strongly enhanced in cells by depleted of CDK12. U2OS cells were transfected with control (CTL) and CDK12-specific siRNA (48 hr) and the PARP inhibitor, Olaparib (12 hr) at different concentrations, as indicated. Total PARylation and auto-PARylation of PARP1 were monitored in total lysates by immunoblot using anti-PARP1 antibody. (B) PARP1 activity is modestly enhanced in cells depleted of CHK1. U2OS cells were transfected with control (CTL) and CHK1-specific siRNAs as described in (A). Total lysates were analyzed by immunoblot, using the indicated antibodies. (C) CRIF1 regulates global levels of PARP activity in a manner opposite to CDK12. U2OS cells were transfected with control (CTL) or CRIF1-specific siRNA for 48 hr, and total lysates were analyzed by immunoblot with the indicated antisera. (D) Schematic diagram illustrates the role of CDK12 and CRIF in distinct DNA damage pathways at the left. At the right, knockdown of CDK12 sensitizes U2OS cells to the PARP inhibitor, Olaparib. Following siRNA transfection, U2OS cells were treated with Olaparib (Ola) 1 μM or Doxorubicin (Dox) 0.1 μM, or both, for 12 hr before measuring cell viability. (E) Oxidative stress disrupts the binding of CRIF1 to CDK12. Top panel: U2OS cells were treated with tBHQ (tert-Butylhydroquinone) 100 μM for 4 h, and cell extracts were analyzed by co-immunoprecipitation with anti-CDK12 antisera for association of CRIF1 or CCNK. Bottom panel: Oxidative stress, but not EGFR signaling, impairs the CRIF1-CDK12 interaction in U2OS cells. (F) Oxidative stress activates the CDK12 pathway. The total cell lysate from (E) was analyzed by immunoblot, using the indicated antibodies. NRF2 and GAPDH levels were monitored as a marker for oxidative stress and as a loading control, respectively.

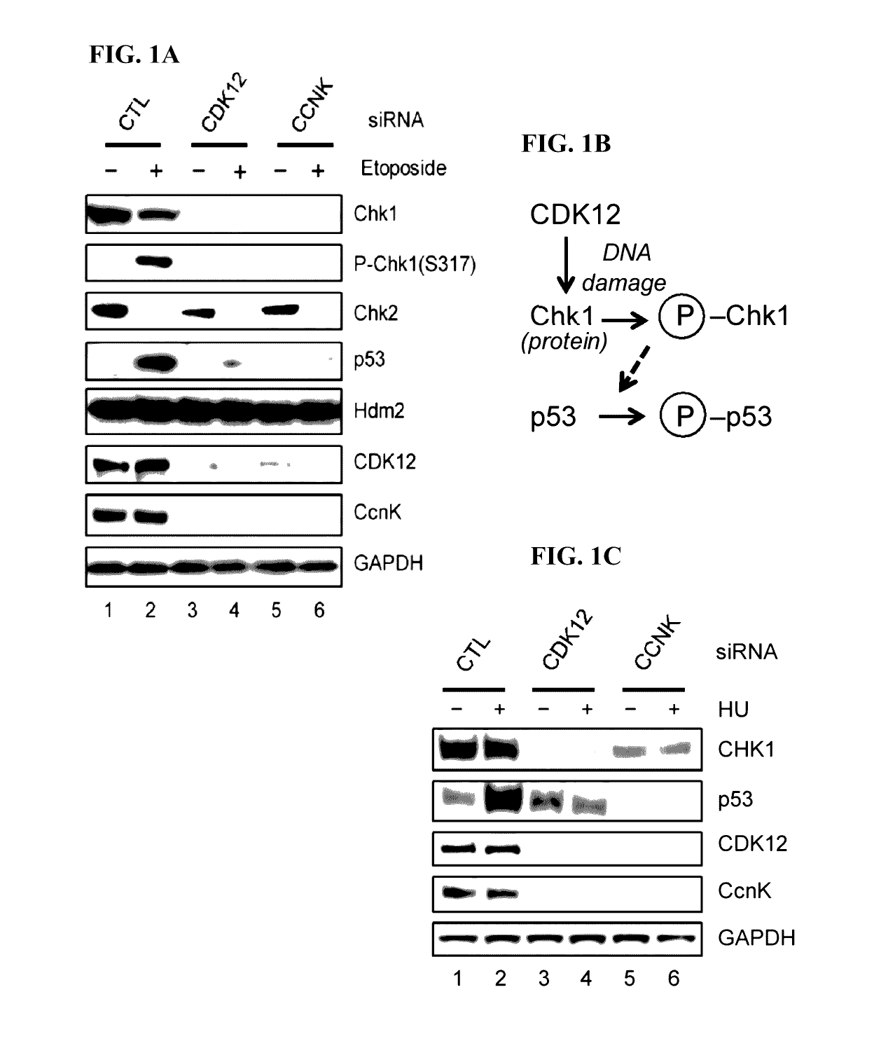

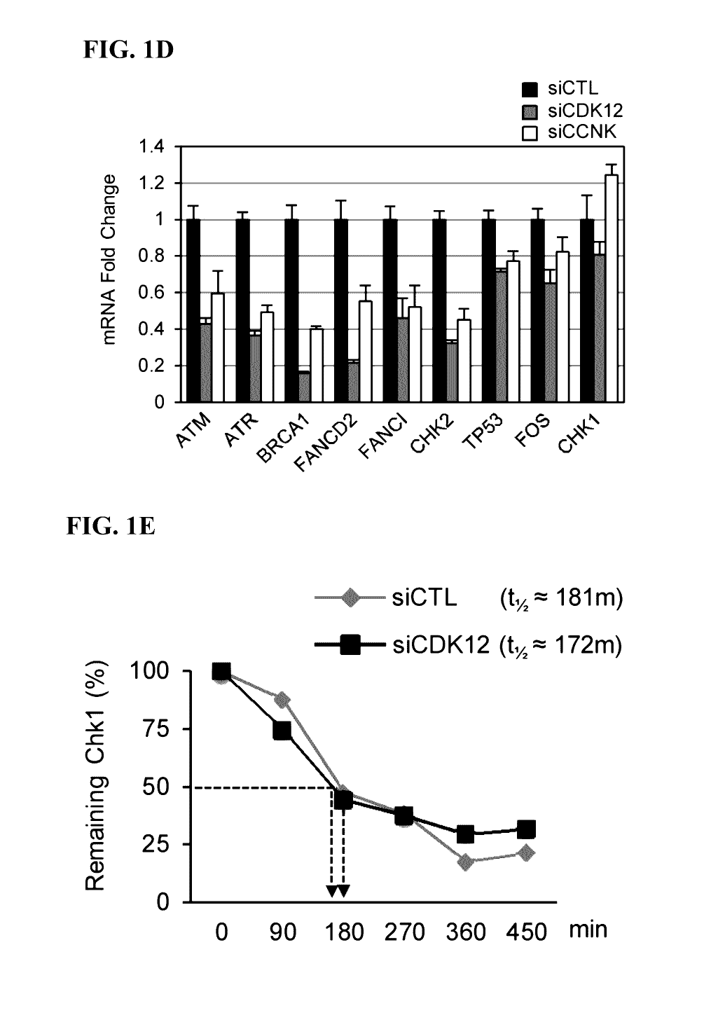

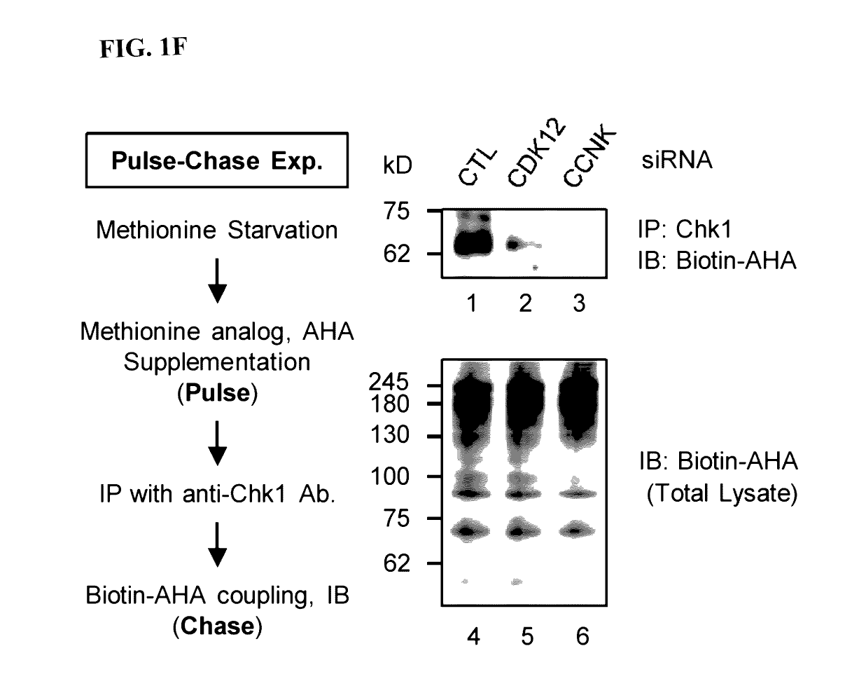

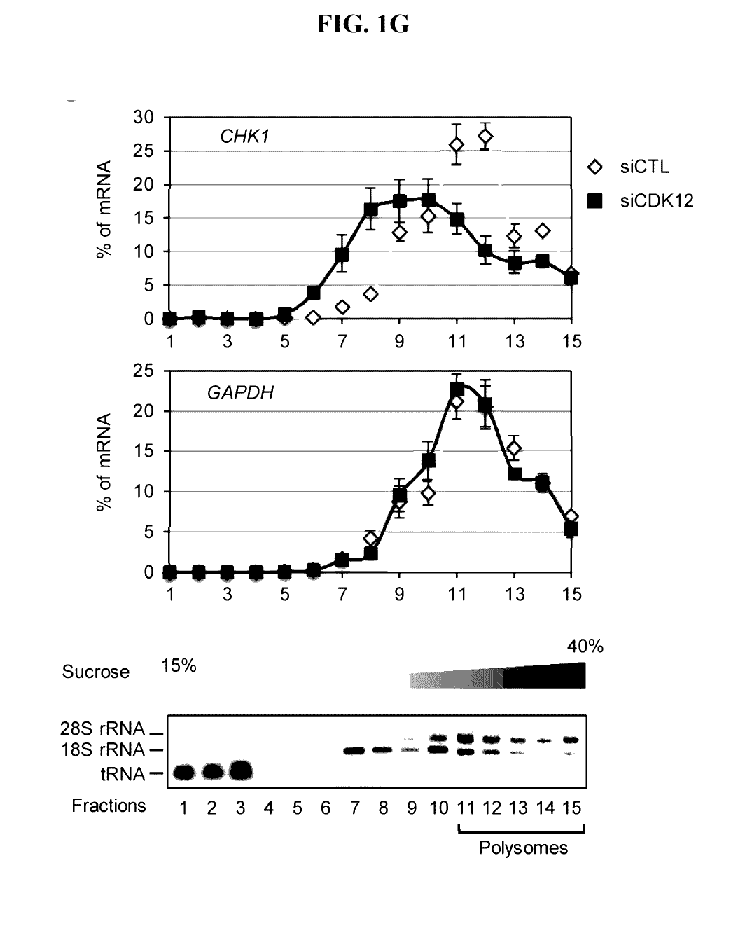

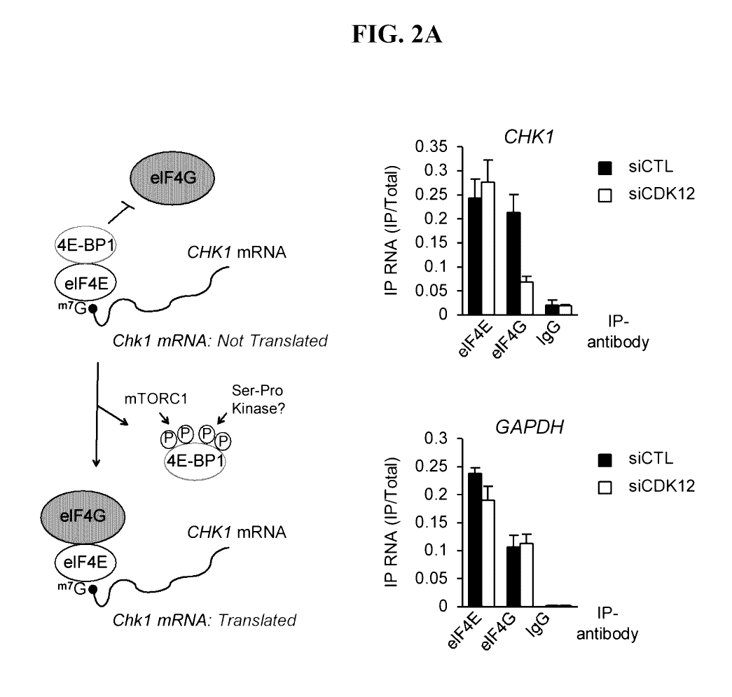

FIGS. 8A-8F. CDK12 Selectively Regulates the Biosynthesis of CHK1 Protein, (A) Transcription of CHK1 under genotoxic stress is unaffected by CDK12 depletion. Shown is a qRT-PCR analysis of U2OS cells treated with control (siCTL), CDK12, or CCNK-specific siRNAs. Total RNA was extracted and subjected to qRT-PCR analysis with gene specific primers, and normalized to GAPDH expression. (B) CHK1 is necessary for the stabilization of p53 by genotoxic stress. Immunoblot analysis of U2OS cells after treatment with Etoposide or DMSO control. Two different siRNAs targeting CHK1 were tested, as indicated. (C) Failure of p53 to be stabilized by DNA damage in cells depleted of CDK12. Shown is an analysis of the stability of the p53 protein in U2OS cells depleted of CDK12. After control or CDK12-specific siRNA transfection, cells were treated with cycloheximide to block protein expression. Levels of p53 protein were measured from total cell lysate by immunoblot using p53-specific antibody (left panel). Quantification of signal intensity corresponding to p53 protein was analyzed and charted in the graph (right panel). The half-life of p53 protein was plotted from the graph. (D) CHK1 protein stability is unaffected by CDK12 depletion. CHK1 protein stability was measured as in (C). These data are plotted in FIG. 1E. (E) The efficiency of CHK1 mRNA termination is unchanged in CDK12 knockdown cells. A schematic represents 3′ end of the CHK1 gene (gDNA), and its pre-, or mature mRNA. The polyA (AATAAA) and cleavage (CA) signals were targeted by gene-specific primers (GSPs). At the bottom, total RNAs extracted from U2OS cells that were depleted of CDK12 or CCNK, and analyzed by reverse transcription with each of the indicated gene-specific primer. qPCR analysis was carried out to determine the Pass-Through (PT) ratio, as shown in the graphs. (F) CDK12 does not affect the cytosolic transport of CHK1 mRNA in vivo. Shown is a qRT-PCR analysis of cytosolic mRNA fractions from U2OS cells transfected with control, CDK12, and CCNK-specific siRNAs, as indicated.

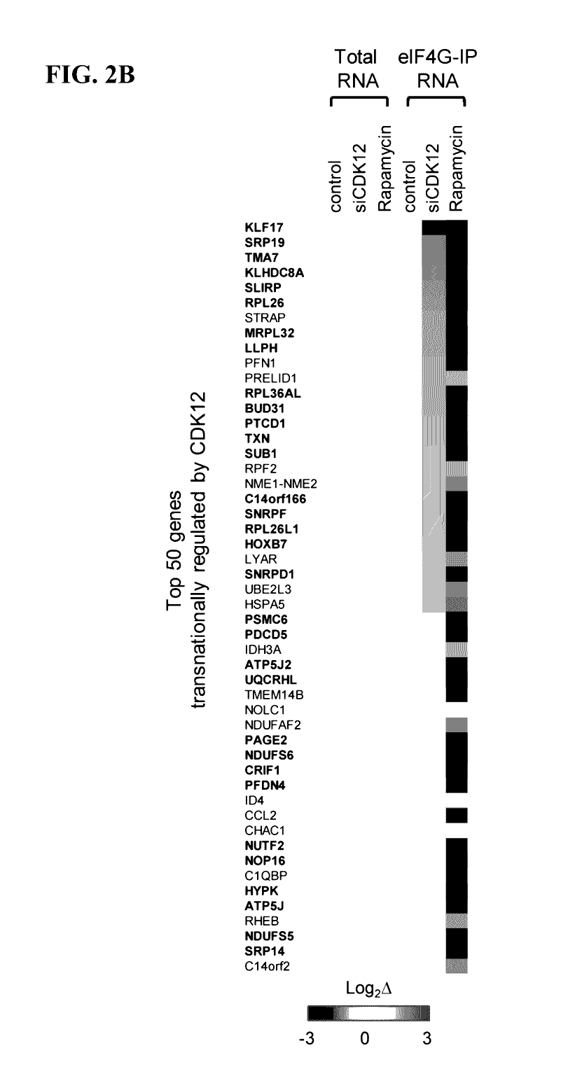

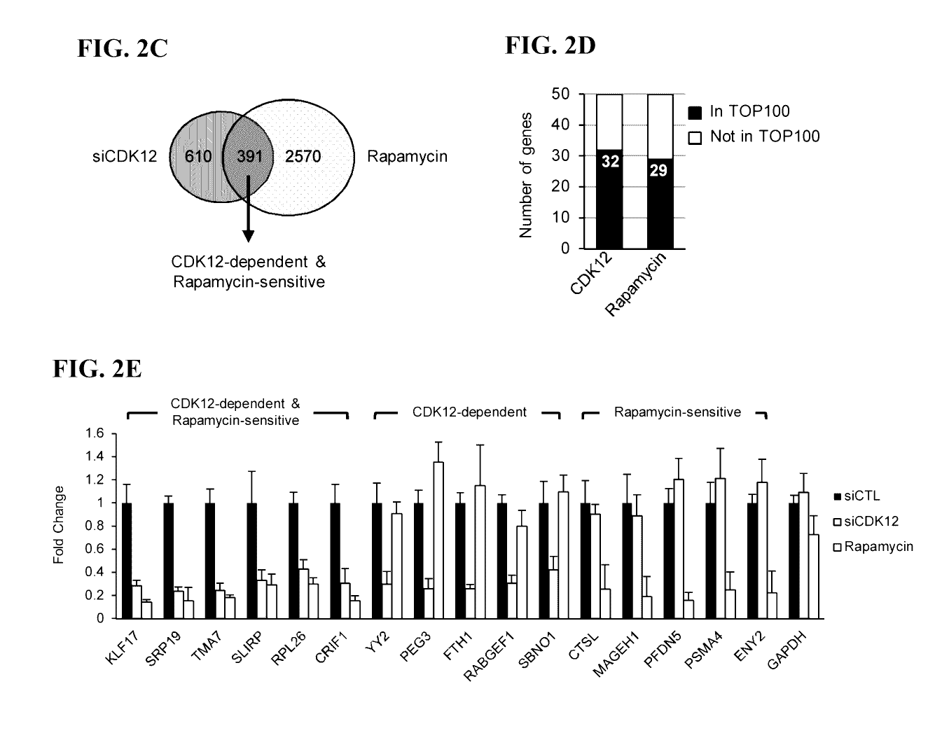

FIGS. 9A-9E. Cdk12 Regulates the Binding of eIF4G to CHK1 mRNA. (A) Knockdown of CDK12 or CCNK in U2OS cells was carried out using gene-specific siRNAs (si a; black) or control siRNA (CTL), and protein levels were analyzed by immunoblot of total cell lysates using the indicated antibodies. (B) List of GO categories of the top CDK12 transcriptional gene targets in U2OS cells. (C) The top diagram shows the results of RNA immunoprecipitation (RIP-) and total RNA (RNA-seq) analyses. The genes (n=113) which showed changes in both eIF4G-binding (SD; n=4) and total RNA levels (SD; n=2) in CDK12-depleted U2OS cells were excluded from further analysis, in order to focus on genes that are solely regulated at the level of translation. The remaining 1001 genes were considered for further analysis. The same analysis was applied to Rapamycin-sensitive genes in U2OS cells, as indicated at the bottom, and identified 2961 genes that could be further characterized as translation targets. (D) List of the top 50 mRNAs that associate with eIF4G in a Rapamycin-responsive manner, as identified by RIP-seq analysis from FIG. 2B. Changes in RNA abundance are shown in Log 2 scale. Total RNA changes of those top 50 genes are also shown in parallel. (E) Grouping of mRNA targets from the RIP-seq analysis. The top five genes were selected as representative of each category. The average mRNA abundance change in either total RNA or eIF4G-bound mRNA is shown in Log 2 scale. (F) List of GO categories for top genes that are translationally-regulated by either CDK12, Rapamycin, or both, in U2OS cells.

FIGS. 10A-10D. CDK12 Promotes Cotranscriptional Loading of 4E-BP1 onto Nascent mRNAs at Target Gene Promoters In Vivo, (A) RNA synthesis is required to recruit both eIF4E and 4E-BP1, but not CDK12 or RNAPII CTD-Ser2, to chromatin at target gene promoters. U2OS cells were treated with α-amanitin for 12 hr to block nascent transcription, and CUP experiments were carried out using the indicated antisera. The genomic positions of the CHK1 gene for q-PCR assays are shown in the graph. (B) The tested CDK12 target genes contain detectable RNAPII CTD-Ser2P occupancy at the promoter. Several putative CDK12 target genes identified by ChIP-seq analysis of U2OS cells were analyzed, using the indicated antisera. (C) CDK12 is required for loading of eIF4G onto target mRNAs. RNA immunoprecipitation and qRT-PCR analyses were carried out with extracts from control or CDK12-depleted U2OS cells. (D) Immunoblot analysis for CDK12 target genes identified in (C).

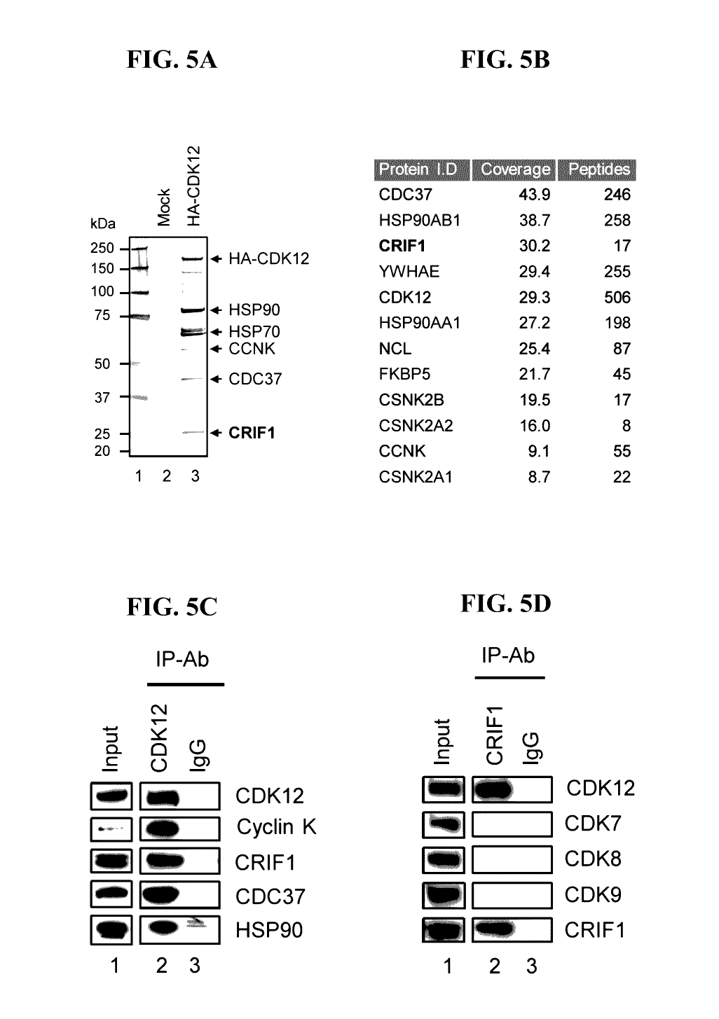

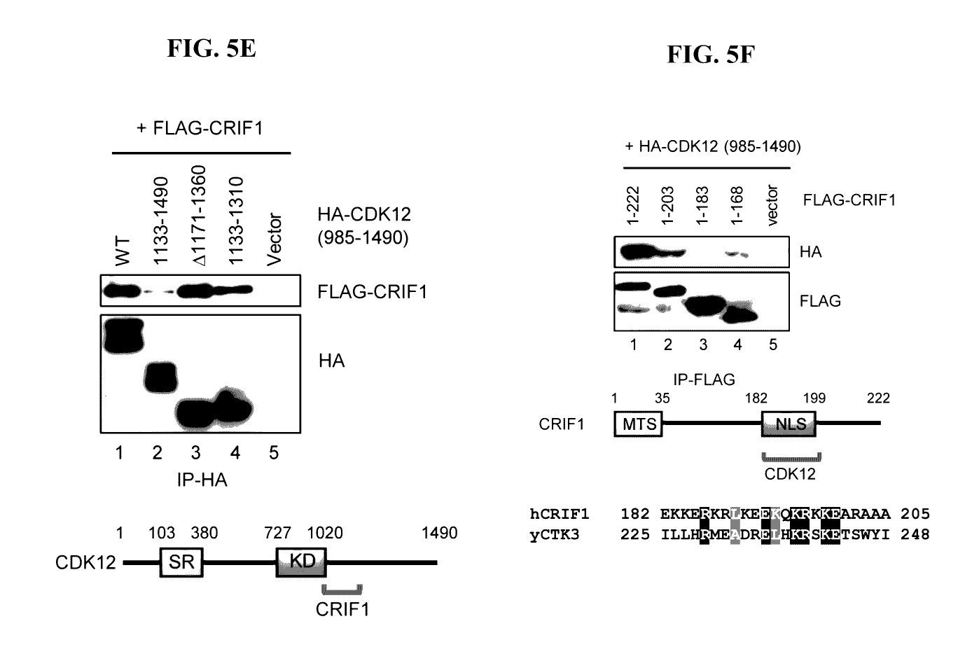

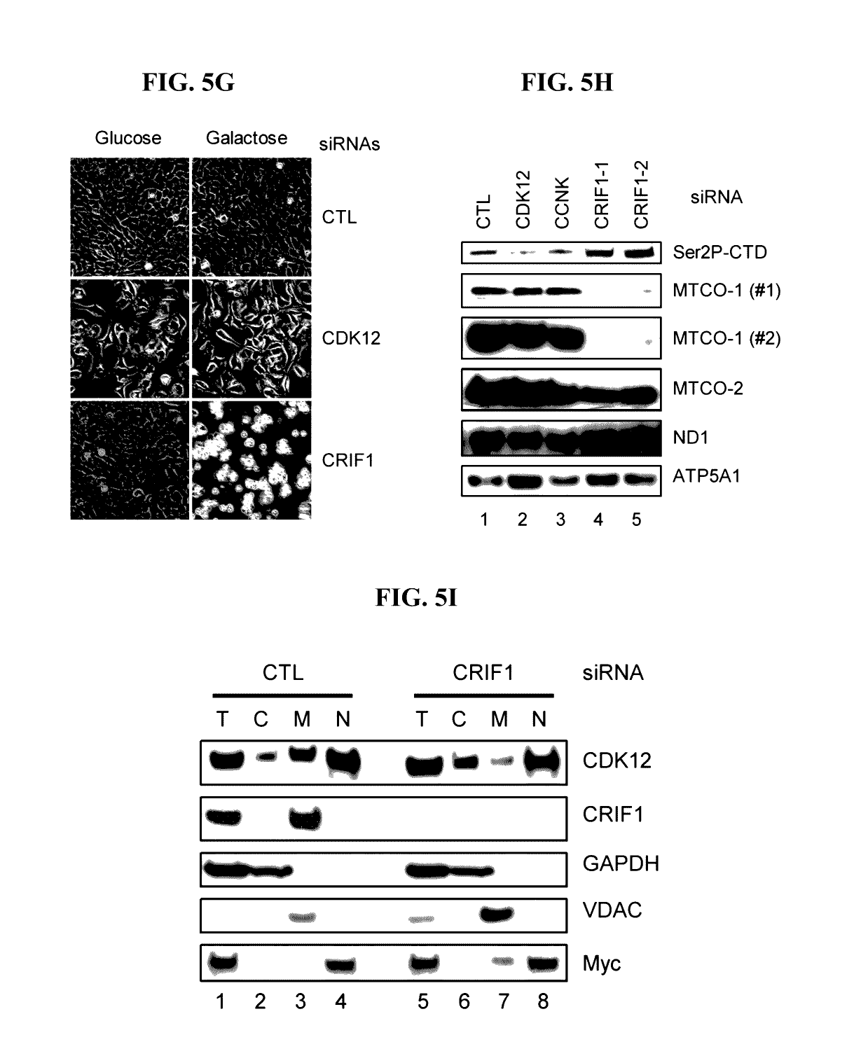

FIGS. 11A-11D. The Mitochondrial OX-PHOS Regulator, CRIF1, is a Subunit of the Human CDK12 Kinase Complex. (A) CRIF1 binds specifically to the CDK12 kinase, and not other nuclear CDKs. The N-terminal mitochondria-targeting sequence (MTS) can be bypassed by the addition of an N-terminal FLAG-tag on the CRIF1 protein, and induces high levels of CRIF1 to be localized to the nucleus. The ability of nuclear FLAG-CRIF1 to associate with various transcription-associated CDKs was analyzed by co-immunoprecipitation and immunoblot analysis, using the indicated CDK-specific antibodies. (B) CRIF1 binds to the C-terminal domain of CDK12. The indicated HA-CDK12 fragments were expressed together with full-length FLAG-CRIF1 in HEK293 cells. Co-immunoprecipitation and immunoblot analysis was carried out using the indicated antibodies. (C) CRIF1 is necessary for ATP production from oxidative phosphorylation. U2OS cells depleted of CCNK in parallel with control siRNA transfection was shown with indicted media (see also FIG. 5G). (D) A schematic model indicating that the binding to CDK12 can mask the CRIF1 nuclear localization sequence (NLS). The N-terminal MTS (mitochondria-targeting sequence) of CRIF1 can then localize CDK12 to the mitochondria.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [7158-96173-01_Sequence_Listing, Dec. 2, 2016, ASCII text file 18.0 KB], which is incorporated by reference herein. In the accompanying sequence listing:
SEQ ID NOs: 1-3 are CRIF1 polypeptides.
SEQ ID NO: 4 is a full length CRIF1 protein.
SEQ ID NOs: 5 and 6 are nuclear localization sequences.
SEQ ID NO: 7 is a CDK12 polypeptide.
SEQ ID NO: 8 is a human RNA polymerase II polypeptide.
SEQ ID NO: 9 is a CRIF1 polypeptide.
SEQ ID NO: 10 is a CTK3 polypeptide.
SEQ ID NO: 11 is a 4EBP1 polypeptide.
SEQ ID NO: 12 is a CDK consensus polypeptide.
SEQ ID NOs: 13-80 are primer sequences.

### DETAILED DESCRIPTION

Existing small molecule inhibitors of transcription-associated CTD kinases, such as flavopiridol, which predominantly targets Cdk9, or THZ1, which is directed against Cdk7, have also been shown to modestly inhibit Cdk12 kinase activity. However highly potent and specific small molecule inhibitors of Cdk12 are not commercially available. The novel interaction between CRIF1 and Cdk12 disclosed herein provides an effective and selective approach to inhibit Cdk12 kinase activity in vivo. The peptide sequence of the Cdk12-interacting domain within CRIF1 (aa184-222; or derivatives thereof) can be used to combat resistance to many types of cancer chemotherapy and immunotherapy. The CRIF polypeptides interact specifically with Cdk12 and potently inhibit its kinase activity. Moreover, CRIF1 does not bind to other Cdks, including Cdk7, Cdk8, Cdk9, nor was binding to Cdk2 detected. Consequently CRIF1 provides a new approach to target endogenous Cdk12, and block its role in mTORC1-dependent translation and coupled expression of DNA repair proteins.

By virtue of its role in DNA repair and homologous recombination, Cdk12 has been directly implicated in enabling cancers to survive exposure to PARP inhibitors. Thus, loss of Cdk12 protein, or inactivation of Cdk12 kinase activity, leads to genome instability and renders cells more sensitive to genotoxic insults. In addition to BRCA1, other Cdk12 targets, such as ATR and histones, are involved in multiple DNA repair pathways, including mismatch repair. Importantly, defects in mismatch repair have been found to confer sensitivity of cells to cancer immunotherapy. Consequently, CRIF1-based inhibitors of Cdk12 could be used to increase the overall effectiveness of cancer immunotherapy treatments, such as the use of PD-1 and PD-L1 monoclonal antibodies. Consistent with its antagonistic role to Cdk12, CRIF1 levels dropped in paclitaxel-resistant ovarian cancer cells, providing another route to upregulate Cdk12 kinase activity. The data disclosed herein provide evidence that over-expression of CRIF1 or peptides corresponding to the region of CRIF1 that binds Cdk12 (for example, aa 183-222 or aa 184-222), or small molecules that mimic the CRIF1:Cdk12 interaction, selectively inhibit Cdk12 activity in cancer cells and render these cells more responsive to chemo- and immunotherapies. Moreover, selective inhibition of Cdk12 enables PARP inhibitors to function in otherwise non-responsive cancers that express wild-type BRCA1 and/or BRCA2 proteins, thereby significantly expanding the utility of these drugs for multiple cancer types. The surprising discovery that Cdk12 cooperates with mTORC1 to phosphorylate 4EBP1 and control translation of target mRNAs, including Chk1, provides evidence that that Cdk12 inhibitors, such as the polypeptides disclosed herein, could augment the activity of existing mTORC1 and Chk1 inhibitors that are currently used or in clinical trials for a variety of different cancers.

### TERMS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided, along with particular examples:

Administration:

To provide or give a subject an agent, for example, a composition that includes or encodes a polypeptide disclosed herein, by any effective route. Exemplary routes of administration include, but are not limited to, oral, injection (such as subcutaneous, intramuscular, intradermal, intraperitoneal, intratumoral, and intravenous) and transdermal (e.g., topical).

Agent:

Any substance or any combination of substances that is useful for achieving an end or result; for example, a substance or combination of substances useful for decreasing or reducing a tumor in a subject. In some embodiments, the agent is a chemotherapeutic agent, toxin or an agent that reduces or mimics the interaction of CRIF1 and Cdk12. The skilled artisan will understand that particular agents may be useful to achieve more than one result.

Aptamer:

Single stranded nucleic acid molecules (such as DNA or RNA) that bind a specific target agent (such as a protein or small organic molecule) with high affinity and specificity (e.g., as high as 10⁻¹⁴ M), and upon binding to the target, the ss nucleic acid molecule undergoes a conformational change and forms a tertiary structure. They are typically around 15 to 60 nt in length, but some are longer (e.g., over 200 nt). Thus, in some examples, aptamers are at least 15 nt, at least 20 nt, at least 25 nt, at least 30 nt, at least 50 nt, at least 60 nt, at least 75 nt, at least 100 nt, at least 150 nt, at least 200 nt, such as 15 to 250 nt, 15 to 200 nt, or 20 to 50 nt.

Aptamers are known in the art and have been obtained through a combinatorial selection process called systematic evolution of ligands by exponential enrichment (SELEX) (see for example Ellington et al., Nature 1990, 346, 818-822; Tuerk and Gold Science 1990, 249, 505-510; Liu et al., Chem. Rev. 2009, 109, 1948-1998; Shamah et al., Acc. Chem. Res. 2008, 41, 130-138; Famulok, et al., Chem. Rev. 2007, 107, 3715-3743; Manimala et al., Recent Dev. Nucleic Acids Res. 2004, 1, 207-231; Famulok et al., Acc. Chem. Res. 2000, 33, 591-599; Hesselberth, et al., Rev. Mol. Biotech. 2000, 74, 15-25; Wilson et al., Annu. Rev. Biochem. 1999, 68, 611-647; Morris et al., Proc. Natl. Acad. Sci. U.S.A. 1998, 95, 2902-2907). In such a process, DNA or RNA molecules that are capable of binding a target molecule of interest are selected from a nucleic acid library consisting of 10¹⁴-10¹⁵ different sequences through iterative steps of selection, amplification and mutation. Aptamers that are specific to a wide range of targets from small organic molecules such as adenosine, to proteins such as thrombin, and even viruses and cells have been identified (Liu et al., Chem. Rev. 2009, 109, 1948-1998; Lee et al., Nucleic Acids Res. 2004, 32, D95-D100; Navani and Li, Curr. Opin. Chem. Biol. 2006, 10, 272-281; Song et al., TrAC, Trends Anal. Chem. 2008, 27, 108-117). The affinity of the aptamers towards their targets can rival that of antibodies, with dissociation constants in as low as the picomolar range (Morris et al., Proc. Natl. Acad. Sci. U.S.A. 1998, 95, 2902-2907; Green et al., Biochemistry 1996, 35, 14413-14424).

Breast Cancer:

A neoplastic condition of breast tissue that can be benign or malignant. The most common type of breast cancer is ductal carcinoma. Ductal carcinoma in situ is a non-invasive neoplastic condition of the ducts. Lobular carcinoma is not an invasive disease but is an indicator that a carcinoma may develop. Infiltrating (malignant) carcinoma of the breast can be divided into stages (I, IIA, IIB, IIIA, IIIB, and IV).

Breast carcinomas lose the typical histology and architecture of normal breast glands. Generally, carcinoma cells overgrow the normal cells and lose their ability to differentiate into glandular like structures. The degree of loss of differentiation in general is related to the aggressiveness of the tumor. For example, “in situ” carcinoma by definition retains the basement membrane intact, whereas as it progresses to “invasive”, the tumor shows breakout of basement membranes. Thus one would not expect to see, within breast carcinomas, staining of a discrete layer of basal cells as seen in normal breast tissue. For a discussion of the physiology and histology of normal breast and breast carcinoma, see Ronnov-Jessen, L., Petersen, 0. W. & Bissell, M. J. Cellular changes involved in conversion of normal to malignant breast: importance of the stromal reaction (see, for example, Physiol Rev 76, 69-125, 1996).

Breast cancers can be divided into groups based on their expression profiles. Basal-type carcinomas usually are negative for expression of estrogen receptor (ER) and negative for expression of HER2 (erbB2) and progesterone receptor (PR), and thus are referred to as “triple-negative breast cancers” or “TNBC.” This type of breast cancer is also denoted ER⁻/HER2⁻/PR⁻ and represents about 15-20% of all breast cancer, and generally cannot be treated using Her2 targeted or estrogen targeted therapies. It is believed that the aggressive nature of this cancer is correlated with an enrichment for cancer stem cells (CSC) with a CD44⁺CD24^{-/lo} phenotype. In some embodiments, basal carcinomas are negative for expression of progesterone receptor (PR), positive for expression of epidermal growth factor receptor (EGFR), and positive for expression of cytokeratin 5 (CK5). This phenotype is denoted as follows: ER⁻/PR⁻/HER2⁻/CK5⁺/EGFR⁺.

Cancer:

A malignant tumor that has undergone characteristic anaplasia with loss of differentiation, increase rate of growth, invasion of surrounding tissue, and is capable of metastasis. For example, thyroid cancer is a malignant tumor that arises in or from thyroid tissue, and breast cancer is a malignant tumor that arises in or from breast tissue (such as a ductal carcinoma). Residual cancer is cancer that remains in a subject after any form of treatment given to the subject to reduce or eradicate the cancer. Metastatic cancer is a tumor at one or more sites in the body other than the site of origin of the original (primary) cancer from which the metastatic cancer is derived. Cancer includes, but is not limited to, solid tumors.

Cyclin Dependent Kinase (Cdk):

A family of serine-threonine protein kinases that bind cyclin and are present in eukaryotes. The consensus sequence for the phosphorylation site in the amino acid sequence of a CDK substrate is [S/T]PX[K/R] (SEQ ID NO: 6), where S/T* is the phosphorylated serine or threonine, P is proline, X is any amino acid, K is lysine, and R is arginine. Most of the known cyclin-CDK complexes regulate the progression through the cell cycle.

Cdk12 was initially identified in cDNA screens for cell cycle regulators, and was initially named CRKRS. Human Cdk12 is a 1490-amino acid protein, with a conserved central CTD kinase domain and degenerate RS domains in the N- and C-terminal regions. Cdk12 was phosphorylates CTD of RNAPII. Based on the interaction of Cdk12 with overexpressed Cyclin L (CycL), CycL was reported to be its regulatory subunit. However, other studies have reported that the endogenous Drosophila Cdk12 and human Cdk12 do not associate with CycL, but rather with CycK. In humans (and likely in other higher organisms), CycK binds Cdk12 in two separate complexes.

The CycK/Cdk12 complex phosphorylates Ser2 in the CTD of RNAPII RNAPII directs the transcription of protein coding genes. The transcription process includes several stages, including preinitiation complex formation, promoter clearance, pausing, productive elongation, and termination. This transcription cycle is tightly linked to the co-transcriptional maturation of nascent transcripts, including pre-mRNA splicing and polyadenylation. RNAPII contains an unstructured CTD with repeats of the evolutionarily conserved heptapeptide, Y1S2P3T4S5P6S7, where individual serines (Ser2, 5, and 7), threonine, and tyrosine can be phosphorylated.

Exemplary Cdk12 sequences are disclosed in: GENBANK® ACCESSION No. NM_016507, VERSION NM_016507.3 GI:568599828; ACCESSION No. NP_057591, VERSION NP_057591.2 GI:157817023, ACCESSION No. NM_015083 VERSION NM_015083.2 GI:568599813, and ACCESSION No. NP_055898, VERSION NP_055898.1 GI:157817073, all incorporated by reference herein as available on Dec. 3, 2015.

Chemotherapeutic Agent:

Any chemical agent with therapeutic usefulness in the treatment of diseases characterized by abnormal cell growth. For example, chemotherapeutic agents are useful for the treatment of cancer, including ovarian and breast cancer. In one embodiment, a chemotherapeutic agent is a radioactive compound. In one embodiment, a chemotherapeutic agent is a biologic, such as a monoclonal antibody.

In particular examples, such chemotherapeutic agents are administered in combination with an inhibitor of the interaction of CR1F1 and Cdk12. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993; Chabner and Longo, Cancer Chemotherapy and Biotherapy: Principles and Practice (4th ed.). Philadelphia: Lippincott Willians & Wilkins, 2005; Skeel, Handbook of Cancer Chemotherapy (6th ed.). Lippincott Williams & Wilkins, 2003). Combination chemotherapy is the administration of more than one agent to treat cancer.

Consists of:

With regard to a polypeptide, a polypeptide that consists of a specified amino acid sequence does not include any additional amino acid residues, nor does it include additional non-peptide components, such as lipids, sugars or labels.

Conservative Variants:

“Conservative” amino acid substitutions are those substitutions that do not substantially affect or decrease an activity or antigenicity of an antigenic epitope of CR1F1 or CDK12. Specific, non-limiting examples of a conservative substitution include the following examples:

| Original Residue | Conservative Substitutions |
|---|---|
| Al | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variant also includes the use of a substituted amino acid in place of an unsubstituted parent amino acid, provided that antibodies raised to the substituted polypeptide also immunoreact with the unsubstituted polypeptide, and/or that the substituted polypeptide retains the function of the unsubstituted polypeptide. Non-conservative substitutions are those that reduce an activity or antigenicity.

Contacting:

Placement in direct physical association, for example solid, liquid or gaseous forms. Contacting includes, for example, direct physical association of fully- and partially-solvated molecules.

Decrease or Reduce:

To reduce the quality, amount, or strength of something; for example a reduction in tumor burden. In one example, a therapy reduces a tumor (such as the size or volume of a tumor, the number of tumors, the metastasis of a tumor, or combinations thereof), or one or more symptoms associated with a tumor, for example as compared to the response in the absence of the therapy. In a particular example, a therapy decreases the size or volume of a tumor, the number of tumors, the metastasis of a tumor, or combinations thereof, subsequent to the therapy, such as a decrease of at least 10%, at least 20%, at least 30%, at least 40%, at least 50%, at least 60%, at least 70%, at least 80%, or at least 90% (for example relative to an amount before the therapy, or in the absence of therapy). Such decreases can be measured using the methods disclosed herein.

Degenerate Variant:

A polynucleotide encoding a polypeptide that includes a sequence that is degenerate as a result of the genetic code. There are 20 natural amino acids, most of which are specified by more than one codon. Therefore, all degenerate nucleotide sequences are included in this disclosure as long as the amino acid sequence of the polypeptide encoded by the nucleotide sequence is unchanged.

DNA (Deoxyribonucleic Acid):

A long chain polymer which includes the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal (termination codon). The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a protein, or a fragment thereof, encompasses both the sense strand and its reverse complement. Thus, for instance, it is appropriate to generate probes or primers from the reverse complement sequence of the disclosed nucleic acid molecules.

Isolated:

An “isolated” biological component (such as a nucleic acid molecule, protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Linker and Linked:

The terms “conjugating,” “joining,” “bonding,” “labeling” or “linking” refer to making two molecules into one contiguous molecule; for example, linking two polypeptides into one contiguous polypeptide, or covalently attaching another molecule to a polypeptide. The linkage can be either by chemical or recombinant means. “Chemical means” refers to a reaction between the polypeptide and the other molecule such that there is a covalent bond formed between the two molecules to form one molecule.

In some embodiments, a linker is an amino acid sequence that covalently links two polypeptide domains. For example, such linkers can be included in the between the CRIF1 polypeptides disclosed herein and a nuclear localization sequence, to provide rotational freedom to the linked polypeptide domains and thereby to promote proper domain folding. By way of example, in a recombinant polypeptide comprising a CRIF1 polypeptide and a nuclear localization sequence, linker sequences can be provided between them, such as a polypeptide comprising CRIF1-linker-nuclear localization peptide. Linker sequences, which are generally between 2 and 25 amino acids in length, are well known in the art and include, but are not limited to, the glycine(4)-serine spacer (GGGGS×3) (SEQ ID NO: 8) described by Chaudhary et al., Nature 339:394-397, 1989.

Nuclear Localization Sequence:

An amino acid sequence that targets a protein for import into the cell nucleus by nuclear transport. Typically, this signal consists of one or more short sequences of positively charged lysines or arginines exposed on the protein surface. Different nuclear localized proteins may share the same NLS.

Ovarian Cancer:

Cancer that forms in tissues of the ovary (one of a pair of female reproductive glands in which the ova, or eggs, are formed). Most ovarian cancers are either ovarian epithelial carcinomas (cancer that begins in the cells on the surface of the ovary) or malignant germ cell tumors (cancer that begins in egg cells).

Poly (ADP-Ribose) Polymerase 1 (PARP1):

An enzyme that modifies nuclear proteins by ADP-ribosylation. The modification is dependent on DNA and is involved in the regulation of cellular processes such as differentiation, proliferation, and tumor transformation. PARP1 also plays a role in the regulation of the molecular events involved in the recovery of cell from DNA damage. An exemplary PARP1 nucleic acid sequence is disclosed in GENBANK® Accession No. NM_001618.3, May 18, 2014, incorporated herein by reference. An exemplary PARP1 protein sequence is disclosed in GENBANK® Accession No. NP_001609.2, incorporated herein by reference. A PARP inhibitor is an agent that significantly decreases the activity of the PARP1 enzyme as measured with decreased PARylation of different proteins, most commonly PARP1 protein itself. Detection of inhibition of PARylation can be detected using Western Blot or other assays such as ELISA.

Peptide Modifications:

Polypeptides include synthetic embodiments of peptides described herein. In addition, analogs (non-peptide organic molecules), derivatives (chemically functionalized peptide molecules obtained starting with the disclosed peptide sequences) and variants (homologs) of these peptides can be utilized in the methods described herein. Each peptide of this disclosure is comprised of a sequence of amino acids, which may be either L- and/or D-amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C₁-C₁₆ alkyl or dialkyl amino or further converted to an amide.

Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this invention to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of a polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165-174 and Principles of Pharmacology, Munson (ed.) 1995, Ch. 102, for descriptions of techniques used in CADD. Also included are mimetics prepared using such techniques.

Pharmaceutically Acceptable Carriers:

The pharmaceutically acceptable carriers provided herein are conventional. Remington's Pharmaceutical Sciences, by E. W. Martin, Mack Publishing Co., Easton, Pa., 15th Edition (1975), describes compositions and formulations suitable for pharmaceutical delivery of the therapeutic agents herein disclosed.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polynucleotide:

The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

Polypeptide or Peptide:

A polymer in which the monomers are amino acid residues that are joined together through amide bonds. The amino acids included in a polypeptide may be subject to post-translational modification (e.g., glycosylation or phosphorylation). A polypeptide or peptide can be between 3 and 52 amino acids in length. In one embodiment, a polypeptide or peptide is 30 to 52 amino acids in length. In several embodiments, a polypeptide or peptide is at most 52 amino acids in length, for example, 30, 35, 40, 45, or 50 amino acids in length.

Plurality:

Two or more of a molecule, such as 2, 3, 4, 5, 6, 7, 8, 9, 10, 11, 12 or more of a molecule.

Promoter:

An array of nucleic acid control sequences which direct transcription of a nucleic acid. A promoter includes necessary nucleic acid sequences near the start site of transcription, such as, in the case of a polymerase II type promoter, a TATA element. In one embodiment, a promoter includes an enhancer. In another embodiment, a promoter includes a repressor element. In these embodiments, a chimeric promoter is created (a promoter/enhancer chimera or a promoter/repressor chimera, respectively). Enhancer and repressor elements can be located adjacent to, or distal to the promoter, and can be located as much as several thousand base pairs from the start site of transcription. Examples of promoters include, but are not limited to the SV40 promoter, the CMV enhancer-promoter, and the CMV enhancer/β-actin promoter. Both constitutive and inducible promoters are included (see e.g., Bitter et al., Methods in Enzymology 153:516-544, 1987). Also included are those promoter elements which are sufficient to render promoter-dependent gene expression controllable for cell-type specific, tissue-specific, or inducible by external signals or agents; such elements may be located in the 5′ or 3′ regions of the gene. Promoters produced by recombinant DNA or synthetic techniques can also be used to provide for transcription of the nucleic acid sequences.

Recombinant:

A recombinant nucleic acid is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, e.g., by genetic engineering techniques.

Sequence Identity:

The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a CRIF1 polypeptide or Cdk12 will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide, such as a CRIF1 polypeptide, are typically characterized by possession of at least 75%, for example at least 80%, sequence identity counted over the full length alignment with the amino acid sequence of the polypeptide using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and can possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Tumor, Neoplasia, Malignancy or Cancer:

A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma and CNS tumors (such as a glioma, astrocytoma, medulloblastoma, craniopharyrgioma, ependymoma, pinealoma, hemangioblastoma, acoustic neuroma, oligodendroglioma, meningioma, neuroblastoma and retinoblastoma).

Tumor Burden:

The total volume, number, metastasis, or combinations thereof of tumor or tumors in a subject.

Therapeutically Effective Amount:

The amount of an agent (such as a polypeptide, a nucleic acid encoding the polypeptide) that alone, or together with one or more additional agents, induces the desired response, such as, for example, induction of an immune response and/or treatment of a tumor in a subject. Ideally, a therapeutically effective amount provides a therapeutic effect without causing a substantial side effects in the subject.

In one example, a desired response is to decrease the size, volume, or number (such as metastases) of a tumor in a subject. For example, the agent or agents can decrease the size, volume, or number of tumors by a desired amount, for example by at least 5%, at least 10%, at least 15%, at least 20%, at least 25%, at least 30%, at least 50%, at least 75%, at least 90%, or at least 95% as compared to a response in the absence of the agent.

Therapeutically effective amounts also can be determined through various in vitro, in vivo or in situ immunoassays. The disclosed agents can be administered in a single dose, or in several doses, as needed to obtain the desired response. However, the therapeutically effective amount can be dependent on the source applied, the subject being treated, the severity and type of the condition being treated, and the manner of administration.

Treating or Treatment:

A therapeutic intervention (e.g., administration of a therapeutically effective amount of a polypeptide, nucleic acid molecule, or composition including the polypeptide or nucleic acid molecule that ameliorates a sign or symptom of a disease or pathological condition related to a disease (such as a tumor). Treatment can also induce remission or cure of a condition, such as a tumor. In particular examples, treatment includes preventing a tumor, for example by inhibiting the full development of a tumor, such as preventing development of a metastasis or the development of a primary tumor. Prevention does not require a total absence of a tumor.

Reducing a sign or symptom associated with a tumor can be evidenced, for example, by a delayed onset of clinical symptoms of the disease in a susceptible subject (such as a subject having a tumor which has not yet metastasized), a reduction in severity of some or all clinical symptoms of the disease, a slower progression of the disease (for example by prolonging the life of a subject having tumor), a reduction in the number of relapses of the disease, an improvement in the overall health or well-being of the subject, or by other parameters well known in the art that are specific to the particular tumor.

Vector:

A nucleic acid molecule as introduced into a host cell, thereby producing a transformed host cell. A vector may include nucleic acid sequences that permit it to replicate in a host cell, such as an origin of replication. A vector may also include one or more selectable marker gene and other genetic elements known in the art. Vectors include plasmid vectors, including plasmids for expression in gram negative and gram positive bacterial cell. Exemplary vectors include those for expression in E. coli. Vectors also include viral vectors, such as, but are not limited to, retroviral, pox, adenoviral, herpes virus, alpha virus, baculovirus, Sindbis virus, vaccinia virus and poliovirus vectors.

Under Conditions Sufficient for:

A phrase that is used to describe any environment that permits a desired activity. In one example the desired activity is formation of an immune complex. In particular examples the desired activity is treatment of a tumor.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, “A or B” is intended to include “A” or “B” and both “A and B” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Overview

To better understand the mechanism of CDK12 action, it is important to define the composition of the native complex, identify other substrates for the kinase that are important for genome stability, and determine how CDK12 kinase activity is regulated. The S. cerevisiae homolog of CDK12, CTD Kinase I (CTDK-I), is a trimeric complex containing the catalytic subunit, CTK1, an activating cyclin, CTK2, and a stimulatory subunit, CTK3, which stabilizes and recruits the complex to target genes upon completion of the nascent mRNA cap structure. In yeast, CTK1 is not essential for growth in glucose, but has a metabolic role and is crucial for switching between nutrient sources. In addition to its role in transcription, yeast CTK1 can also affect mRNA translation initiation and elongation (Coordes et al., 2015; Rither and StrdBer, 2007). Human CDK12 is a large protein, and like other cyclin dependent kinases, contains a regulatory loop adjacent to the catalytic site that is essential for catalytic activity (Bosken et al., Nat Commun 5, 3505, 2014; Dixon-Clarke et al., Sci Rep 5, 17122, 2015). The CDK12 N-terminus contains multiple RS (Arginine- and Serine-rich) domains, indicating that it may also contact RNA, or RNA-interacting proteins, to affect splicing or 3′-end processing of nascent mRNAs.

It was determined that human CDK12 is essential for expression of the CHK1 protein kinase, which stabilizes the p53 tumor suppressor in response to DNA damage. Unexpectedly, the results indicated that CDK12 was required for mRNA translation, but not transcription, of the CHK1 gene. Moreover, it was observed that CDK12 phosphorylates the 4E-BP1 translation repressor (Qin et al., Cell Cycle 15, 781-786, 2016) at two Ser-Pro sites (S65 and T70), both in vivo and in vitro. In addition, CDK12 cooperated with mTORC1 to phosphorylate and release 4E-BP1 and promote binding of eIF4G to a subset of mRNAs important for translation and DNA damage repair. The analysis indicated that CDK12 can act at responsive gene promoters to facilitate the exchange of 4E-BP1 and eIF4G on nascent mRNAs.

Furthermore, the nuclear and mitochondrial OX-PHOS regulatory protein, CRIF1 (Chung et al., J Biol Chem 278, 28079-28088, 2003; Kim et al., Cell Metab 16, 274-283, 2012; Ryu et al., PLoS Genet 9, e1003356, 2013; Nagar et al., PLoS One 9, e98670, 2014; Ran et al., PLoS One 9, e85328, 2014), was identified as a near-stoichiometric regulatory subunit of human CDK12:CCNK complexes. CRIF1 selectively inhibits phosphorylation of CDK12 CTD and 4E-BP1, in vivo and in vitro, and sequestered a fraction of CDK12 in the mitochondria in U2OS cells. Furthermore, the interaction between CDK12 and CRIF1 was regulated by oxidative stress. These events provide a surprising coordination between RNAPII and 4E-BP1 phosphorylation that marks CHK1 and other responsive mRNAs for enhanced translation to ensure genome stability and cell survival under stress.

Based on these observations, CRIF1 polypeptides are provided that inhibit Cdk12 activity or nuclear localization in cells, and can be used use to treat or prevent cancer (for example to augment chemotherapy or immunotherapy).

### CRIF1 Polypeptides that Inhibit Cdk12 Activity

Methods are disclosed herein that utilize agents that inhibit Cdk12 activity and/or Cdk12 nuclear localization in cancer cells. In some embodiments, the methods utilize a Growth Arrest and DNA Damage-Inducible Proteins-Interacting Protein 1 (GADD45GIP1 or CRIF1) polypeptide. In other embodiments, the agent is a small molecule. Complete elimination of Cdk12 activity and/or nuclear localization not required. In some examples, the CRIF polypeptide reduces Cdk12 activity and/or nuclear localization by at least 25%, at least 30%, at least 40%, at least 50%, at least 75%, at least 90%, at least 95%, at least 98%, at least 99%, or 100%.

These agents mimic the interaction of CRIF1 with Cdk12 in a cell, and thus reduce Cdk12 activity. In some non-limiting examples the agents can bind the same region of Cdk12 bound by endogenous CRIF1 in the cell, and thus can inhibit binding of the endogenous CRIF1 polypeptide to Cdk12.

In some embodiments, the CRIF1 polypeptide includes the amino acid sequence set forth as

KKERKRL KEEKQKRKKE (SEQ ID NO: 1, amino acids 184-200 of CRIF1) wherein the polypeptide is at most 52 amino acids in length. Additional amino acids can be added to the polypeptide (in addition to SEQ ID NO: 1), for the polypeptide to function, specifically additional consecutive amino acids of CRIF1. Thus, in some embodiments, 1, 2, 3, 4, 5, 6, 7, 9, 10, 11, 12, 13, 14, 15, 16, 17, 18, 19, 20, 21 or 22 corresponding consecutive amino acids of CRIF1 are included at the C-terminus. In further embodiments, 1, 2, 3, 4, 5, 6, 7, 8, 9, 10 or more corresponding amino acids of CRIF1 are included at the N-terminus. In some embodiments, this short polypeptide is non-naturally occurring. In particular embodiments, additional heterologous amino acid sequences, not from CRIF1, are included in the polypeptide.

In some examples, the polypeptide includes or consists of an amino acid sequence having at least 80%, at least 85%, at least 90%, at least 92%, at least 95%, at least 98% or at least 99% sequence identity to SEQ ID NO: 3 or SEQ ID NO: 2. Thus, for example, SEQ ID NO: 1 can include at least 1, at least 5, or at least 10 conservative amino acid substitutions (such as 1, 2, 3, 4, 5, 6, 7, 9, 10, 11, 12, 13, 14, 15, 16, 17, 18, 19, or 20 conservative amino acid substitutions), and is in some examples 52 aa in length. In other embodiments, the polypeptide includes the amino acid sequence of SEQ ID NO: 1, and is about least at least 90%, at least 92%, at least 95%, at least 98% or at least 99% identical to SEQ ID NO: 3 or SEQ ID NO: 2. In other embodiments, the polypeptide consists of the amino acid sequence of SEQ ID NO: 1.

In some embodiments, the CRIF1 polypeptide includes the amino acid sequence set forth as

₁₈₄KKERKRL KEEKQKRKKE ARAAALAAAV AQDPAASGAP SS₂₂₂ (SEQ ID NO: 2, amino acids 184-222 of CRIF1), wherein the polypeptide is at most 52 amino acids in length. Additional amino acids can be added to the polypeptide (in addition to SEQ ID NO: 2), for the polypeptide to function, specifically additional consecutive amino acids of CRIF1. Thus, in some embodiments, 1, 2, 3, 4, 5, 6, 7, 9, 10, 11, 12, 13, 14, 15, 16, 17, 18, 19, 20, 21 or 22 corresponding consecutive amino acids of CRIF1 are included at the C-terminus. In further embodiments, 1, 2, 3, 4, 5, 6, 7, 8, 9, 10 or more corresponding amino acids of CRIF1 are included at the N-terminus. In some embodiments, this short polypeptide is non-naturally occurring. In particular embodiments, additional heterologous amino acid sequences, not from CRIF1, are included in the polypeptide. In other embodiments, the polypeptide is 39 amino acids in length. The CRIF1 polypeptide can consist of the amino acid sequence of SEQ ID NO: 2.

In additional embodiments, the polypeptide includes or consists of an amino acid sequence having at least 80%, at least 85%, at least 90%, at least 92%, at least 95%, at least 98% or at least 99% sequence identity to SEQ ID NO: 2, and is in some examples 39 amino acids in length. Thus, for example, SEQ ID NO: 2 can include at least 1, at least 5, or at least 10 conservative amino acid substitutions (such as 1, 2, 3, 4, 5, 6, 7, 9, 10, 11, 12, 13, 14, 15, 16, 17, 18, 19, or 20 conservative amino acid substitutions).

In further embodiments, the polypeptide can include the amino acid sequence:

PRSARFQELLQDLEKKERKLKEEKQKRKKEARAAALAAAVAQDPAASG APSS (SEQ ID NO: 3, amino acids 170-222 of CRIF1). In some examples, the polypeptide includes or consists of an amino acid sequence having at least 80%, at least 85%, at least 90%, at least 92%, at least 95%, at least 98% or at least 99% sequence identity to SEQ ID NO: 3. Thus, for example, SEQ ID NO: 3 can include at least 1, at least 5, or at least 10 conservative amino acid substitutions (such as 1, 2, 3, 4, 5, 6, 7, 9, 10, 11, 12, 13, 14, 15, 16, 17, 18, 19, or 20 conservative amino acid substitutions), and is in some examples 52 amino acids in length.

In some embodiments, the polypeptide is at most 52 amino acids in length. Thus, the polypeptide can be 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or 52 amino acids in length. In a specific non-limiting example, the polypeptide consists of the amino acid sequence of SEQ ID NO: 3.

Combinations of these polypeptides are also of use. Thus, in some embodiments, compositions are provided that include one or more of these polypeptides (such one two or all three of SEQ ID NOS: 1, 2, and 3) in a pharmaceutically acceptable excipient.

In some embodiments, the polypeptides do not include the full length CRIF1 amino acid sequence:

(SEQ ID NO: 4)

MAASVRQARS LLGVAATLAP GSRGYRARPP PRRRPGPRWP
DPEDLLTPRW QLGPRYAAKQ FARYGAASGV VPGSLWPSPE
QLRELEAEER EWYPSLATMQ ESLRVKQLAE EQKRREREQH
IAECMAKMPQ MIVNWQQQQR ENWEKAQADK ERRARLQAEA
QELLGYQVDP RSARFQELLQ DLEKKERKRL KEEKQKRKKE
ARAAALAAAV AQDPAASGAP SS

In vivo, CRIF1 is a component in the large subunit of mitoribosome, and plays a role in the translation of mitochondrial oxidative phosphorylation polypeptides in mammalian mitochondria. CRIF1 interacts with nascent OXPHOS polypeptides and the mitochondrial-specific chaperone Tid1. CRIF1 binds Cdk12, and inhibits Cdk12 activity. In some non-limiting examples, the disclosed polypeptides bind the same site on Cdk12 that interacts with endogenous CRIF1, and thereby inhibit Cdk12 activity.

In some embodiments, the polypeptides are modified to prevent degradation. For example, the polypeptides can be amidated or include non-naturally occurring amino acids. Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C₁-C₁₆ alkyl or dialkyl amino or further converted to an amide.

Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups.

The disclosed polypeptides can be fused to other molecules, including polypeptides. In some embodiments, and additional nuclear localization sequence linked to the CRIF1 polypeptide (such as any of SEQ ID NOS; 1, 2 and 3). The disclosed polypeptides can be fused to a cell penetrating polypeptide, see Sakhrani and Padh, Drug Des. Ther. 7: 585-599, incorporated herein by reference. The disclosed polypeptides can be linked to another nuclear localization sequence, such as small peptides from viruses that show nuclear localization, such as KKKRKV (SEQ ID NO: 5) peptide from SV40 large T antigen or KRPAAT KKAGQAKKKKL (SEQ ID NO: 6) from nucleoplasmin, or a REV-1 nuclear localization sequence. Nuclear localization polypeptides from the VP2 protein of chick anemia virus are of use, see U.S. Published Patent Application No. 2013/0023643. Thus, in some embodiments, the polypeptide can also be ligated to an aptamer, a tat protein, or a nuclear localization sequence. In another embodiment, the polypeptide can be ligated to Rev-1. A viral vector can be used to achieve nuclear delivery, see below.

The disclosed CRIF1 polypeptides inhibit Cdk12 activity. Without being bound by theory, the region of human CDK12 that interacts with CRIF1 includes amino acids 985-1132. This partially overlaps the CDK12 kinase active site that is required for kinase activity.

(SEQ ID NO: 7)
EFSFIPSAALDLLDHMLTLDPSKRCTAEQTLQSDFLKDVELSKMAPPDLP
HWQDCHELWSKKRRRQRQSGVVVEEPPPSKTSRKETTSGTSTEPVKNSSP
APPQPAPGKVESGAGDAIGLADITQQLNQSELAVLLNLLQSQTDLSIP.

Mimics of the CRIF1/Cdk12 interaction are of use in the methods disclosed herein. In some embodiments, a mimetics inhibits the binding of endogenous CRIF1 to Cdk12. The mimetics inhibit Cdk12 activity and/or nuclear localization.

Agents of use include molecules that are identified from large libraries of natural product or synthetic (or semi-synthetic) extracts or chemical libraries. The molecule can be small molecules, such as compounds of less than about a 900 dalton molecular weight, such as those with a size on the order of 10⁻⁹ m. The screening methods that detect a decrease in the interaction of wild-type CRIF1 and Cdk12 are useful for identifying compounds from a variety of sources for activity, see the examples section below. Screening methods for Cdk12 activity and/or nuclear localization are also of use. The initial screens may be performed using a diverse library of compounds, a variety of other compounds and compound libraries. Using these assays for a read-out, small molecules can be identified from combinatorial libraries, natural product libraries, or other small molecule libraries. In addition, small molecule antagonists can be identified as compounds from commercial sources, as well as commercially available analogs of identified inhibitors.

The precise source of test extracts or compounds is not critical to the identification of the agents of use. Accordingly, small molecules can be identified from virtually any number of chemical extracts or compounds. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N. H.) and Aldrich Chemical (Milwaukee, Wis.). Small molecules can be identified from synthetic compound libraries that are commercially available from a number of companies including Maybridge Chemical Co. (Trevillet, Cornwall, UK), Comgenex (Princeton, N. J.), Brandon Associates (Merrimack, N. H.), and Microsource (New Milford, Conn.). Small molecules can be identified from a rare chemical library, such as the library that is available from Aldrich (Milwaukee, Wis.). Small molecules can be identified in libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Natural and synthetically produced libraries and compounds are readily modified through conventional chemical, physical, and biochemical means.

Useful compounds may be found within numerous chemical classes, though typically they are organic compounds, including small organic compounds. In some embodiments, the small molecule antagonists are organic compounds have a molecular weight of more than 50 yet less than about 900 daltons, such as less than about 750 or less than about 350 daltons can be utilized in the methods disclosed herein. Exemplary classes include heterocycles, peptides, saccharides, steroids, and the like. The compounds may be modified to enhance efficacy, stability, pharmaceutical compatibility, and the like. In several embodiments, a compound of use has a Kd for Cdk12 of less than InM, less than 10 nm, less than 1 μM, less than 10 μM, or less than 1 mM.

### Nucleotides, Expression Vectors and Host Cells

Nucleic acids encoding one or more CRIF1 polypeptides are provided. These polynucleotides include DNA, cDNA and RNA sequences which encode the polypeptide(s) of interest. Nucleic acid molecules encoding these peptides can readily be produced, using the amino acid sequences provided herein, and the genetic code. In addition, one of skill can readily construct a variety of clones containing functionally equivalent nucleic acids, such as nucleic acids which differ in sequence but which encode the same effector molecule.

In some examples, a nucleic acid molecule encoding a CRIF1 polypeptide encodes a peptide having at least 80%, at least 85%, at least 90%, at least 92%, at least 95%, at least 98% or at least 99% sequence identity to SEQ ID NO: 2 or SEQ ID NO: 3.

Nucleic acid sequences encoding one or more CRIF1 polypeptides can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template.

Exemplary nucleic acids including sequences encoding a disclosed CRIF1 polypeptide can be prepared by cloning techniques. Examples of appropriate cloning and sequencing techniques, and instructions sufficient to direct persons of skill through cloning are found in Sambrook et al., supra, Berger and Kimmel (eds.), supra, and Ausubel, supra. Product information from manufacturers of biological reagents and experimental equipment also provide useful information. Such manufacturers include the SIGMA Chemical Company (Saint Louis, Mo.), R&D Systems (Minneapolis, Minn.), Pharmacia Amersham (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), Invitrogen (San Diego, Calif.), and Applied Biosystems (Foster City, Calif.), as well as many other commercial sources known to one of skill.

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

Once a nucleic acid molecule encoding a CRIF1 polypeptide can be used to express the corresponding protein in a recombinantly engineered cell such as bacteria, plant, yeast, insect or mammalian cell using a suitable expression vector. One or more DNA sequences encoding the CRIF1 polypeptide can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Polynucleotide sequences encoding a CRIF1 polypeptide can be operatively linked to expression control sequences (e.g., a promoter). An expression control sequence operatively linked to a coding sequence is ligated such that expression of the coding sequence is achieved under conditions compatible with the expression control sequences. The expression control sequences include, but are not limited to appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

The polynucleotide sequences encoding the CRIF1 polypeptide can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art.

Transformation of a host cell with recombinant DNA may be carried out by conventional techniques. Where the host is prokaryotic, such as E. coli, competent cells which are capable of DNA uptake can be prepared from cells harvested after exponential growth phase and subsequently treated by the CaCl₂ method. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with polynucleotide sequences encoding a CRIF1 polypeptide, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the one or more polypeptides (see for example, Eukaryotic Viral Vectors, Cold Spring Harbor Laboratory, Gluzman ed., 1982). One of skill in the art can readily use expression systems such as plasmids and vectors of use in producing proteins in cells including higher eukaryotic cells such as the COS, CHO, HeLa and myeloma cell lines.

In some embodiments, one or more polynucleotides encoding a CRIF1 polypeptide are included in one or more viral vectors. Examples of suitable viral vectors include retrovirus vectors, pox vectors, adenoviral vectors, herpes virus vectors, alpha virus vectors, baculovirus vectors, Sindbis virus vectors, vaccinia virus vectors lentivirus and poliovirus vectors. Basic techniques for preparing recombinant DNA viruses containing a heterologous DNA sequence are known in the art. Such techniques involve, for example, homologous recombination between the viral DNA sequences flanking the DNA sequence in a donor plasmid and homologous sequences present in the parental virus (Mackett et al., 1982, Proc. Natl. Acad. Sci. USA 79:7415-7419).

Viral vectors can be prepared encoding a CRIF1 polypeptide. A number of viral vectors have been constructed, including polyoma, SV40 (Madzak et al., J. Gen. Virol., 73:15331536, 1992), adenovirus (Berkner, Cur. Top. Microbiol. Immunol., 158:39-6, 1992; Berliner et al., Bio Techniques, 6:616-629, 1988; Gorziglia et al., J. Virol., 66:4407-4412, 1992; Quantin et al., Proc. Nad. Acad. Sci. USA, 89:2581-2584, 1992; Rosenfeld et al., Cell, 68:143-155 1992; Wilkinson et al., Nucl. Acids Res., 20:2233-2239, 1992; Stratford-Perricaudet et al., Hum. Gene Ther., 1:241-256, 1990), vaccinia virus (Mackett et al., Biotechnology, 24:495-499, 1991), adeno-associated virus (Muzyczka, Curr. Top. Microbiol. Immunol., 158:91-123, 1992; On et al., Gene, 89:279-282, 1990), herpes viruses including HSV and EBV (Margolskee, Curr. Top. Microbiol. Immunol., 158:67-90, 1992; Johnson et al., J. Virol., 66:29522965, 1992; Fink et al., Hum. Gene Ther., 3:11-19, 1992; Breakfield et al., Mol. Neurobiol., 1:337-371, 1987; Fresse et al., Biochem. Pharmacol., 40:2189-2199, 1990), Sindbis viruses (Herweijer et al., Human Gene Therapy, 6:1161-1167, 1995; U.S. Pat. Nos. 5,091,309 and 5,2217,879), alphaviruses (Schlesinger, Trends Biotechnol., 11:18-22, 1993; Frolov et al., Proc. Natl. Acad. Sci. USA, 93:11371-11377, 1996) and retroviruses of avian (Brandyopadhyay et al., Mol. Cell Biol., 4:749-754, 1984; Petropouplos et al., J. Virol., 66:3391-3397, 1992), murine (Miller, Curr. Top. Microbiol. Immunol., 158:1-24, 1992; Miller et al., Mol. Cell Biol., 5:431-437, 1985; Sorge et al., Mol. Cell Biol., 4:1730-1737, 1984; Mann et al., J. Virol., 54:401-407, 1985), and human origin (Page et al., J. Virol., 64:5370-5276, 1990; Buchschalcher et al., J. Virol., 66:2731-2739, 1992). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Viral vectors that encode a CRIF1 polypeptide typically include at least expression control element (e.g., a promoter) operationally linked to the nucleic acid sequence encoding the CRIF1 polypeptide. The at least on expression control element is inserted in the poxviral vector to control and regulate the expression of the nucleic acid sequence. Examples of expression control elements of use in these vectors include, but are not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the CRIF1 polypeptide in the host system. The expression vector can contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

Isolation and purification of recombinantly expressed polypeptide can be carried out by conventional means including preparative chromatography and immunological separations. Once expressed, the CRIF1 polypeptide can be purified according to standard procedures, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes. Once purified, partially or to homogeneity as desired, if to be used therapeutically, the polypeptides should be substantially free of endotoxin.

### Therapeutic Methods and Pharmaceutical Compositions

In some embodiments, compositions that include an agent that inhibits Cdk12 activity and/or nuclear localization, such as a CRIF1 polypeptide, a nucleic acid molecule encoding the CRIF1 polypeptide, or a small molecule, as disclosed herein, are administered to a subject having a disease, such as cancer. The cancer can be a solid tumor, such as, but not limited to, ovarian cancer or breast cancer. The breast cancer can be a basal breast carcinoma. In some embodiments, the basal breast carcinoma is negative for expression of estrogen receptor (ER) and negative for expression of HER2 (erbB2) and progesterone receptor (PR), and thus is a triple-negative breast cancer. Subjects with the cancer, such as the ovarian cancer or the breast cancer, can be selected for treatment.

In some embodiments, a subject is selected for treatment that has cancer. In specific non-limiting examples, the method reduces tumor volume and/or metastasis of the cancer. In other embodiments, a subject is selected that is at risk for developing cancer, such as a subject with a specific BRCA1 and/or BRCA2 gene. In some embodiments, cells of the cancer expresses wild-type BRCA1 and/or wild-type BRCA2.

Amounts effective for this use will depend upon the vascularization of the cancer, the general state of the patient's health, the robustness of the patient's immune system, and the particular therapeutic agent used. In one example, a therapeutically effective amount of the composition is that which provides either subjective relief of a symptom(s) or an objectively identifiable improvement, such as a decrease in tumor size or metastasis. In other embodiments, the methods render the cancer more susceptible for treatment with other agents, such, but not limited to, a chemotherapeutic agent that affects base excision repair, a PARP1 inhibitor, a mammalian target of rapamycin (mTOR) inhibitor, a Cdk inhibitor, a checkpoint kinase (CHK1) inhibitor, or an immunotherapeutic agent, such as a PD-1 antagonist. In additional embodiments, one or more of these agents can also be administered to the subject.

In exemplary applications, compositions are administered to a subject having a disease, such as cancer (for example, ovarian or breast cancer), in an amount sufficient to treat the cancer. Administration is sufficient to inhibit cancer growth, reduce metastasis, render the cancer sensitive to other chemotherapeutic agents, or to reduce a sign or a symptom of the tumor. Amounts effective for this use will depend upon the severity of the disease, the general state of the patient's health, and the robustness of the patient's immune system. In one example, a therapeutically effective amount of the compound is that which provides either subjective relief of a symptom(s) or an objectively identifiable improvement as noted by the clinician or other qualified observer.

One or more CRIF1 polypeptides or one or more polynucleotides encoding these peptides, and/or a small molecule that mimics the interaction of CRIF1 and Cdk12, can be administered by any means (see Banga, A., “Parenteral Controlled Delivery of Therapeutic Peptides and Proteins,” in Therapeutic Peptides and Proteins, Technomic Publishing Co., Inc., Lancaster, Pa., 1995) either locally or systemically, such as by intramuscular, subcutaneous, intraperitoneal, intratumoral, or intravenous injection, but even oral, nasal, transdermal, vaginal, or anal administration is contemplated. In one embodiment, administration is by subcutaneous or intramuscular injection. To extend the time during which the polypeptide, small molecule or polynucleotide is available to stimulate an anti-tumor response, the peptide, protein or polynucleotide can be provided as an implant, an oily injection, or as a particulate system. The particulate system can be a microparticle, a microcapsule, a microsphere, a nanocapsule, or similar particle. (see, e.g., Banga, supra).

Controlled release parenteral formulations can be made as implants, oily injections, or as particulate systems. For a broad overview of protein delivery systems, see Banga, Therapeutic Peptides and Proteins: Formulation, Processing, and Delivery Systems, Technomic Publishing Company, Inc., Lancaster, Pa., 1995. Particulate systems include microspheres, microparticles, microcapsules, nanocapsules, nanospheres, and nanoparticles. Microcapsules contain the therapeutic protein as a central core. In microspheres, the therapeutic agent is dispersed throughout the particle. Particles, microspheres, and microcapsules smaller than about 1 μm are generally referred to as nanoparticles, nanospheres, and nanocapsules, respectively. Capillaries have a diameter of approximately 5 m so that only nanoparticles are administered intravenously. Microparticles are typically around 100 m in diameter and are administered subcutaneously or intramuscularly (see Kreuter, Colloidal Drug Delivery Systems, J. Kreuter, ed., Marcel Dekker, Inc., New York, N.Y., pp. 219-342, 1994; Tice & Tabibi, Treatise on Controlled Drug Delivery, A. Kydonieus, ed., Marcel Dekker, Inc. New York, N.Y., pp. 315-339, 1992).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

In another embodiment, provided is a pharmaceutical composition including one or more polynucleotides encoding a CRIF1 polypeptide (such as 1, 2 or 3 of SEQ ID NO: 1, 2 or 3), or vectors including these polypeptides. A therapeutically effective amount of polynucleotide can be administered to a subject in order to treat a tumor, or to render the tumor susceptible to treatment with other chemotherapeutic agents. In one specific, non-limiting example, a therapeutically effective amount of the polynucleotide is administered to a subject to treat ovarian cancer or breast cancer. In other specific non-limiting examples, a therapeutically effective amount of the polynucleotide is administered to a subject to cells of the expresses wild-type BRCA1 and/or wild-type BRCA2. Optionally, the subject can be administered additional chemotherapeutic agents, such as an agent that affects base excision repair, a PARP1 inhibitor, and mTOR inhibitor, and/or a CHK1 inhibitor.

As described above, any nucleotide sequence encoding a polypeptide can be placed under the control of a promoter to increase expression of the molecule. One approach to administration of nucleic acids is direct immunization with plasmid DNA, such as with a mammalian expression plasmid. U.S. Pat. No. 5,880,103 describes several methods of delivery of nucleic acids encoding peptides or other antigens to an organism. The methods include liposomal delivery of the nucleic acids (or of the synthetic peptides themselves.

In another approach to using nucleic acids, a nucleic acid encoding a CRIF1 polypeptide can also be expressed by attenuated viral hosts or vectors or bacterial vectors. Recombinant vaccinia virus, poxvirus, adenovirus, lentivirus, adeno-associated virus (AAV), herpes virus, retrovirus, or other viral vectors can be used to express one or more polypeptides. When a viral vector is utilized, it is desirable to provide the recipient with a dosage of each recombinant virus in the composition in the range of from about 10⁵ to about 10¹⁰ plaque forming units/mg mammal, although a lower or higher dose can be administered.

The composition of recombinant viral vectors can be introduced into a mammal either prior to any evidence of a cancer, or to mediate regression of the disease in a mammal afflicted with the cancer. Examples of methods for administering the composition into mammals include, but are not limited to, exposure of cells to the recombinant virus ex vivo, or injection of the composition into the affected tissue or intravenous, subcutaneous, intradermal or intramuscular administration of the virus. Alternatively the recombinant viral vector or combination of recombinant viral vectors may be administered locally by direct injection into the cancerous lesion in a pharmaceutically acceptable carrier. Generally, the quantity of recombinant viral vector carrying the nucleic acid sequence encoding the CRIF1 polypeptide that is administered is based on the titer of virus particles. An exemplary range to be administered is 10⁵ to 10¹⁰ virus particles per mammal, such as a human.

In one embodiment, a nucleic acid encoding the CRIF1 polypeptide is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites, including tissues in proximity to metastases. Dosages for injection are usually around 0.5 μg/kg to about 50 mg/kg, and typically are about 0.005 mg/kg to about 5 mg/kg (see, for example, U.S. Pat. No. 5,589,466).

In one specific, non-limiting example, a pharmaceutical composition for intravenous administration would include about 0.1 μg to 10 mg of a CRIF1 polypeptide per patient per day. Dosages from 0.1 up to about 100 mg per patient per day can be used. Actual methods for preparing administrable compositions are known or apparent to those skilled in the art and are described in more detail in such publications as Remingtons Pharmaceutical Sciences, 19^{th} Ed., Mack Publishing Company, Easton, Pa., 1995.

Single or multiple administrations of the compositions are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be used.

In some embodiments, the method of treating a subject can be include the use of anti-cancer agents or other therapeutic treatments (such as immunotherapeutic agents). For example, the subject can receive additional therapies (a) prior to, during, or following administration of a therapeutic amount of an agent that mimics the interaction of CRIF1/Cdk12, such as a CRIF1 polypeptide, or (b) prior to, during, or following administration of a therapeutic amount of the agent that mimics the interaction of CRIF1/Cdk12, such as a CRIF1 polypeptide. In some embodiments, the additional agents acts synergistically with the agent that mimics the interaction of CRIF1/Cdk12, such as a CRIF1 polypeptide.

In one example, the subject receives one or more treatments to remove or reduce the tumor prior to administration of a therapeutic amount of one or more agents for treatment of the tumor. For example, the additional agent may include, but is not limited to, a chemotherapeutic agent, an anti-angiogenic agent, or a combination thereof. In another example, at least part of the tumor is surgically or otherwise excised or reduced in size or volume prior to administering the therapeutically effective amount of the antibody or conjugate.

In some embodiments, the subject is administered a therapeutically effective amount of a PARP1 inhibitor. The PARP1 inhibitor can be any compound of interest. In some embodiments, the PARP1 inhibitor is a 4-carboxamido-isoindolinone derivative, see Published U.S. Patent Application No. 2015/0274662, incorporated herein by reference. Additional PARP1 inhibitors are 4-hydroxyquinazoline and its derivatives, Carboxamino-benzimidazole and its derivatives, 4-aminonaphtalimide and its derivatives, PJ34 homologues, and tetracycline derivatives (see Published U.S. Patent Application No. 2011/0098255, incorporated herein by reference). Additional PARP1 inhibitors are tetraaza phenalen-3-one compounds, see Published U.S. Patent Application No. 2009/0098084, incorporated herein by reference. An exemplary PARP1 inhibitors are 4-iodo-3-nitrobenzamide. The synthesis of BA (4-iodo-3-nitrobenzamide) is described in U.S. Pat. No. 5,464,871, which is incorporated herein by reference.

In some embodiments, the PARP1 inhibitor is

olaparib (AZD-2281, LYNPARZA®),

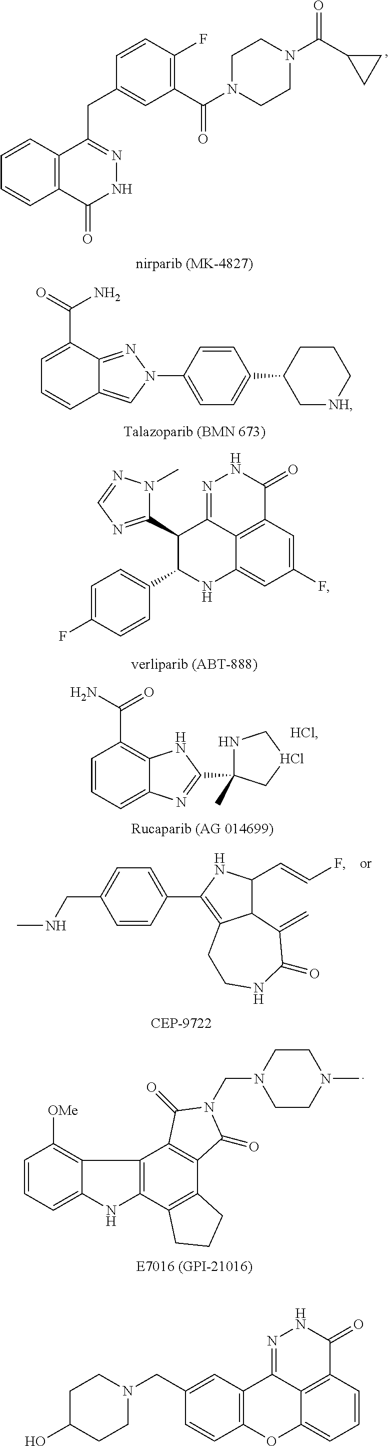

In some non-limiting examples, the PARP1 inhibitor reduces PARP1 activity by at least 90% in a cell with a wild-type PARP1 gene. Clinical development of PARP1 inhibitors follows two distinct approaches: A. targeting cells that are genetically predisposed to die when PARP1 activity is lost; and B. combining PARP1 inhibition with DNA-damaging agents (Rouleau et al., Nature Reviews (2010) 10, 293-301).

In additional embodiments, the methods include administering to the subject a therapeutically effective amount of an mTOR inhibitor or a CHK1 inhibitor. In other embodiments, the methods include administering to the subject a therapeutically effective amount of a Cdk inhibitor. In some embodiments, the methods include administering to the subject a therapeutically effective amount of an mTOR inhibitor such as rapamycin, sirolimus, temsirolimus, everolimus, ridaforolimus, NVPBEZ235, BGT226, XL765, GDC0980, SF1126, PKI587, PFO04691502, GSK2126458, INK128, TORKiCC223, OSI027, AZD8055, AZD2014, and Palomid 529. Illustrative indirect mTOR inhibitors include metformin and AICAR (5-amino-1-˜-D-ribofuranosyl-imidazole-4-carboxamide).

In some embodiments, the methods include administering to the subject a therapeutically effective amount of a CHK1 inhibitor (examples listed in Table 1). In other embodiments, the methods include administering to the subject a therapeutically effective amount of a Cdk inhibitor (examples listed in Table 2).

TABLE 1
Selected Chk1 inhibitors in preclinical or clinical development
(from Br J Clin Pharmacol. 2013 September; 76(3): 358-369.)

| | | | Phase of |
|---|---|---|---|
| Compound name | Company | Other targets | development |
| AZD7762 | AstraZeneca | CDK1, Chk2, CAMK, | Discontinued |
| | | SRC-like kinase | |
| SCH900776/ | Merck | Pim1 | Phase II |
| MK-8776 | | | |
| IC83/LY2603618 | Ely Lilly | Undisclosed | Phase I/II with |
| | | | pemetredex and |
| | | | cisplatin |
| LY2606368 | Ely Lilly | Chk2 | Phase I |
| GDC-0425 | Genentech | Undisclosed | Phase I |
| PF-00477736 | Pfizer | Chk2, VEGFR2, Fms, | Discontinued |
| | | Yes, Flt3, Ret | |
| XL844 | Exelixis | Chk2 | Discontinued |
| CEP-3891 | Cephalon | Undisclosed | Preclinical |
| SAR-020106 | Sareum | Undisclosed | Preclinical |
| CCT-244747 | Sareum | FLT3, Chk2, CDK1 | Preclinical |
| Arry-575 | Array | Undisclosed | Preclinical |

TABLE 2
Exemplary CDK inhibitors.
(from Cancers (Basel). 2014 December; 6(4): 2224-2242)

| | | | In Clinical |
|---|---|---|---|
| | Alternative | Kinases | Development |
| Inhibitor | Names | Inhibited | Yes/No |
| 3α-Amino-5α- | | CDK5 | No |
| androstane | | | |
| 7x | | CDK4, | No |
| | | ARK5 | |
| AG-024322 | | CDK1, | Yes |
| | | CDK2, | |
| | | CDK4 | |
| AMG 925 | | CDK4, | No |
| | | FLT3 | |
| AT7519 | | CDK1, | Yes |
| | | CDK2 | |
| AZD5438 | | CDK1, | Yes |
| | | CDK2, | |
| | | CDK4, | |
| | | CDK5, | |
| | | CDK6, | |
| | | CDK9 | |
| BAY 1000394 | | CDK1, | No |
| | | CDK2, | |
| | | CDK3, | |
| | | CDK4, | |
| | | CDK7, | |
| | | CDK9 | |
| BML-259 | | CDK2, | No |
| | | CDK5 | |
| Compound 1 | | CDK4, | No |
| | | ABL, | |
| | | FGFR1, | |
| | | FYN, KDR, | |
| | | LCK, LYN, | |
| | | SRC | |
| Compound | | CDK4, | No |
| 530 | | CDK4 | |
| CR8 | | CDK2, | No |
| | | CDK5 | |
| Dinaciclib | MK-7965, SCH | CDK1, | Yes |
| | 727965 | CDK2, | |
| | | CDK5, | |
| | | CDK9 | |
| F07#13 | | CDK2, | No |
| | | CDK9 | |
| Fascaplysin | | CDK4, | No |
| | | CDK6 | |
| Flavopiridol | L-868275, HMR- | CDK1, | Yes |
| | 1275, alvocidib, | CDK2, | |
| | NSC-649890 | CDK4, | |
| | | CDK7 | |
| Kenpaullone | NSC 664704, 9- | CDK1, | No |
| | bromopaullone | CDK2, | |
| | | CDK5 | |
| LY2835219 | abemaciclib | CDK4, | Yes |
| | | CDK6 | |
| NBI1 | | CDK2 | No |
| NU2058 | | CDK1, | No |
| | | CDK2 | |
| Olomoucine | | CDK1, | No |
| | | CDK2, | |
| | | CDK5 | |
| P276-00 | | CDK1 | Yes |
| PD-0332991 | | CDK4, | Yes |
| | | CDK6 | |
| PHA-793887 | | CDK1, | Yes (Stopped) |
| | | CDK2, | |
| | | CDK4 | |
| Purvalanol | | CDK1, | No |
| A/B | | CDK2, | |
| | | CDK5 | |
| R547 | Ro-4584820 | CDK1, | Yes |
| | | CDK2, | |
| | | CDK4 | |
| RGB-286638 | | Pan-CDK | No |
| Roscovitine | CY-202, (R)- | CDK2, | Yes |
| | roscovitine, | CDK5 | |
| | seliciclib | | |
| Ryuvidine | | CDK4 | No |
| SNS-032 | BMS-387032 | CDK2, | Yes |
| | | CDK7, | |
| | | CDK9 | |
| SU 9516 | | CDK1, | No |
| | | CDK2 | |
| VMY-1-101 | | CDK2, | No |
| | | CDK5, | |
| | | CDK7 | |
| VMY-1-103 | | CDK2, | No |
| | | CDK5, | |
| | | CDK7 | |

In further embodiments, the methods include administering to the subject a therapeutically effective amount of another chemotherapeutic agent. Examples of chemotherapeutic agents are alkylating agents, antimetabolites, natural products, or hormones and their antagonists. Examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine). Specific non-limiting examples of alkylating agents are temozolomide and dacarbazine. Examples of antimetabolites include folic acid analogs (such as methotrexate), pyrimidine analogs (such as 5-FU or cytarabine), and purine analogs, such as mercaptopurine or thioguanine. Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide). Examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of the most commonly used chemotherapy drugs include ADRIAMYCIN™, ALKERAN™ Ara-C, BiCNU, Busulfan, CCNU, Carboplatinum, Cisplatinum, CYTOXAN™, Daunorubicin, DTIC, 5-FU, Fludarabine, HYDREA™, Idarubicin, Ifosfamide, Methotrexate, Mithramycin, Mitomycin, Mitoxantrone, Nitrogen Mustard, TAXOL™ (or other taxanes, such as docetaxel), Velban, Vincristine, VP-16, while some more newer drugs include Gemcitabine (GEMZAR™), HERCEPTIN™, Irinotecan (CAMPTOSAR™, CPT-11), Leustatin, NAVELBINE™, RITUXAN™, STI-571, TAXOTERE™, Topotecan (HYCAMTIN™), XELODA™ (Capecitabine), Zevelin and calcitriol. Non-limiting examples of immunomodulators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG® (from Imreg of New Orleans, La.), SK&F 106528, and TNF (tumor necrosis factor; Genentech).

Additional therapeutic agents that can be used include microtubule binding agents, DNA intercalators or cross-linkers, DNA synthesis inhibitors, DNA and/or RNA transcription inhibitors, antibodies, enzymes, enzyme inhibitors, gene regulators, angiogenesis inhibitors. These agents (which are administered at a therapeutically effective amount) and treatments can be used alone or in combination. Methods and therapeutic dosages of such agents are known to those skilled in the art, and can be determined by a skilled clinician.

Microtubule binding agent refers to an agent that interacts with tubulin to stabilize or destabilize microtubule formation thereby inhibiting cell division. Examples of microtubule binding agents that can be used in conjunction with the disclosed therapy include, without limitation, paclitaxel, docetaxel, vinblastine, vindesine, vinorelbine (NAVELBINE®), the epothilones, colchicine, dolastatin 15, nocodazole, podophyllotoxin and rhizoxin. Analogs and derivatives of such compounds also can be used and are known to those of ordinary skill in the art. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

Suitable DNA and/or RNA transcription regulators, including, without limitation, actinomycin D, daunorubicin, doxorubicin and derivatives and analogs thereof also are suitable for use in combination with the disclosed therapies. DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof. DNA synthesis inhibitors suitable for use as therapeutic agents include, without limitation, methotrexate, 5-fluoro-5′-deoxyuridine, 5-fluorouracil and analogs thereof. Examples of suitable enzyme inhibitors include, without limitation, camptothecin, etoposide, formestane, trichostatin and derivatives and analogs thereof. Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof.

The method can include administering to the subject a therapeutically effective amount of an immunotherapy. Non-limiting examples of immunomodulators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech). The immunotherpautic agent can be a PD-1 antagonist or a PD-L1 antagonist, such as an antibody that specifically binds PD-1 or PD-L, such as Atezolizumab, MPDL3280A, BNS-936558 (Nivolumab), Pembrolizumab, Pidilizumab, CT011, AMP-224, AMP-514, MEDI-0680, BMS-936559, BMS935559, MEDI-4736, MPDL-3280A, MSB-0010718C. The immunotherpautic agent can also be a CTLA-4, LAG-3, or B7-H3 antagonist, such as Tremelimumab, BMS-986016, and MGA271.

Non-limiting examples of anti-angiogenic agents include molecules, such as proteins, enzymes, polysaccharides, oligonucleotides, DNA, RNA, and recombinant vectors, and small molecules that function to reduce or even inhibit blood vessel growth. Examples of suitable angiogenesis inhibitors include, without limitation, angiostatin K1-3, staurosporine, genistein, fumagillin, medroxyprogesterone, suramin, interferon-alpha, metalloproteinase inhibitors, platelet factor 4, somatostatin, thromobospondin, endostatin, thalidomide, and derivatives and analogs thereof. For example, in some embodiments the anti-angiogenesis agent is an antibody that specifically binds to VEGF (e.g., AVASTIN®, Roche) or a VEGF receptor (e.g., a VEGFR2 antibody). In one example the anti-angiogenic agent includes a VEGFR2 antibody, or DMXAA (also known as Vadimezan or ASA404; available commercially, e.g., from Sigma Corp., St. Louis, Mo.) or both. The anti-angiogenic agent can be bevacizumab, sunitinib, an anti-angiogenic tyrosine kinase inhibitors (TKI), such as sunitinib, xitinib and dasatinib. These can be used individually or in any combination.

Exemplary kinase inhibitors include GLEEVAC®, IRESSA®, and TARCEVA®, sunitinib, sorafenib, anitinib, and dasatinib that prevent phosphorylation and activation of growth factors. Antibodies that can be used include HERCEPTIN® and AVASTIN® that block growth factors and the angiogenic pathway. These can be used individually or in combination.

In some examples, the additional agent is a monoclonal antibody, for example, 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab.

Another common treatment for some types of cancer is surgical treatment, for example surgical resection of the cancer or a portion of it. Another example of a treatment is radiotherapy, for example administration of radioactive material or energy (such as external beam therapy) to the tumor site to help eradicate the tumor or shrink it prior to surgical resection.

The disclosure is illustrated by the following non-limiting Examples.

### EXAMPLES

### Example 1

### Material & Methods

Cell Culture and DNA Transfection:

HEK293 and U2OS cells were cultured in DMEM supplemented with 2 mM L-Glutamine and 10% Fetal Bovine Serum in a CO₂ (5%) incubator at 37° C. G418 selection for stable expression in HEK293 was carried out by addition of G418 at final concentration of 500 μg/mL. For ectopic expression of vector DNA, 5 μg of DNA was transfected into HEK293 cell using Turbo DNAFECTIN™ 3000 (Lamda Biotech) following instructions from the supplier. Cell fractionation, immunoprecipitation and immunoblot procedures are described below.

siRNA Transfection:

The factor-specific siRNAs used were from Life Technologies, and were transfected into cells using the LIPOFECTAMINE® RNAiMAX Transfection Reagent. Experimental analyses were performed 48 h after transfection. For negative control siRNA, the Silencer® Negative Control #1 siRNA was used (Life Technologies). The siRNA sequences used in this study are provided below.

Reverse Transcription and Quantitative RT-PCR:

Total RNA was isolated using the Trizol reagent (Life Technologies), and reverse transcription was performed using 3 μg of RNA extracted using SUPERSCRIPTIII® (Life Technologies) with oligo-dT. PCR measurements to cDNA were performed in triplicate using SYBR® Green master mixes (Life Technologies). Amplification was carried out in the ABI7300 (Applied Biosystems) with a 10 min DNA denaturation step at 95° C., followed by 40 cycles of 15 s at 95° C., 60 s at 60° C. The average of the technical replicates was normalized to GAPDH levels using the comparative CT method. Averages and standard deviation are the result of at least 3 independent experiments. The qRT-PCR primers are listed below.

Affinity Purification of FLAG-Tagged Protein Complexes and MudPIT Analysis:

Stable expression of HA-tagged full-length human CDK12 in HEK293 cells was established using G418 selection. Cells from fifteen 150 mm dishes were extracted using IP buffer (50 mM HEPES-NaOH pH 7.9, 300 mM NaCl, 1% NP-40, 10 mM MgCl₂, 15% glycerol) with protease inhibitors to a final volume of 15 ml, and homogenized by dounce. Extracts were clarified by centrifugation (14,000×g for 15 min at 4° C.). FLAG-M2 beads (Sigma) were incubated with the supernatant in a ratio of 80 μslurry (50% beads in slurry) to 6 mg of total protein for 4 h on a roller at 4° C. The beads were washed in IP-wash buffer (25 mM HEPES-NaOH pH 7.9, 300 mM NaCl, 0.2% NP-40 four times for 3 min at 4° C. and then twice in FLAG-elution buffer (25 mM TRIS®-HCl, pH 7.5, 50 mM NaCl). Finally, the sample was incubated with FLAG peptide (200 μg/ml; Sigma) in elution buffer for 30 min at room temperature with rotation. The MudPIT protein identification methods and analysis are provided below

Determination of Protein Half-Life:

To measure the half-life of endogenous CHK1 and p53 proteins, U20S cells in a 6-well dish were transfected with siRNAs, as indicated in each figure. After 48 h, CHX (cycloheximide, 50 μg/ml at the final) was directly added to cells and incubated for the times indicated in each figure. Cells were washed twice with PBS and lysed in RIPA buffer (50 mM Tris-Cl, pH 7.7, 150 mM NaCl, 1 mM EDTA, 1% NP-40 (v/v), 0.1% SDS(w/v), 0.1% NaDeoxycholate (w/v) to prepare cell extracts. After centrifugation (14,000×g for 15 min at 4° C.), the soluble fraction was subject to SDS-PAGE and immunoblot analysis. The immunoblot signals were analyzed and quantified by ImageJ to obtain the half-life.

Chromatin Immunoprecipitation (ChIP) Experiments:

Briefly, 5×10⁶ cells were twice washed with ice-cold PBS and serially cross-linked with 2 mM DSG (Disuccinimidylglutarate) for 20 min and 1% (v/v) formaldehyde for 10 min at room temperature. The cross-linking reaction was stopped by adding glycine to a final concentration of 0.125 M for 10 min at room temperature. Cells were washed twice with cold PBS. Cell pellets were lysed in 0.5 mL of Lysis buffer (50 mM Tris-HCl, pH 8.0, 10 mM EDTA, 1% SDS (v/v) supplemented with Protease Inhibitor Cocktail (Calbiochem). Chromatin was sonicated to generate DNA-fragments to an average size of 300-500 bp using a Model 505 Sonic Dismembrator (Fisher Scientific) with 5 cycles of 10 s/on and 60 s/off. After centrifugation at 13,000 g for 20 min, immunoprecipitation was performed using 40 μg of chromatin and 2-3 μg of antibody in ChIP buffer (20 mM Tris-HCl pH 8.0, 150 mM NaCl, 2 mM EDTA, and 1% Triton X-100) combined with the Protease Inhibitor Cocktail. After overnight incubation at 4° C., 60 μl slurry of protein A or G agarose was added for 1 hr with rotation. After stepwise wash with wash buffers, immune complexes were eluted in the buffer containing 1% SDS and 100 mM NaHCO₃. Crosslinking was reversed by heating at 65° C. for 4 hr with addition of proteinase K (Sigma). Eluted DNA and 10% of input chromatin were purified using phenol-chloroform extraction followed by isopropanol precipitation or using QIAQUICK® PCR purification (Qiagen), according to the manufacturer instructions. ChIP DNA is analyzed by SYBR® Green master mixes (Life Technologies) using described primer sets. qPCR is carried out in the ABI7300 (Applied Biosystems) with a 10 min DNA denaturation step at 95° C., followed by 40 cycles of 15 s at 95° C., 60 s at 60° C. PCR measurements were performed in triplicate. The average of the technical replicates was normalized to input DNA per set of primer using the comparative CT method. Antibodies were used to precipitate chromatin and ChIP primers were used for qPCR of ChIP DNA.

RNA Immunoprecipitation (RIP) Assay:

All steps were conducted at 4° C. in RNase-free conditions. U20S cells (5×10⁶) were transfected with the indicated siRNAs and washed twice with cold PBS. Cells were collected in 1.5 ml of RNA lysis buffer (25 mM Tris-HCl pH 7.5, 150 mM KCl, 5 mM EDTA, 1% NP-40 (v/v), and freshly added 0.5 mM DTT and 400 U/ml RNase inhibitor (NEB) and homogenized by dounce. After centrifugation at 13,000×g for 20 min, 0.7 ml of clear lysate was transferred to new tubes in addition with antisera (5 μg/IP) for immunoprecipitation. Antibody and lysate mixtures were incubated overnight with rotation. Aliquots of 60 al of protein A or G (50% slurry) were added in the mixture and incubated for additional 2 hr. The agarose slurry was collected and washed three times in 1 ml of RNA Lysis buffer followed by centrifugation at 1,000×g for 2 min. After the final wash, 1 ml of Trizol was used to extract captured RNAs from the immune complexes. After DNA digestion by RNase-free DNase I (NEB), RNA was precipitated by isopropyl alcohol or concentrated using the RNA Clean and Concentrator kit (Zymo Research). Reverse transcription and qRT-PCR for quantitative RNA analysis was carried out as described above.

Cell Culture, DNA Transfection:

HEK293 and U20S cells were cultured in DMEM supplemented with 2 mM L-Glutamine and 10% Fetal Bovine Serum in a CO₂ (5%) incubator at 37° C. G418 selection for stable expression in HEK293 was carried out by addition of G418 at the final concentration of 500 μg/mL or puromycin at the final concentration of 1 μg/ml. For ectopic expression of vector DNA, 5 μg of DNA was transfected into HEK293 cell using Turbo DNAFECTIN™ 3000 (Lamda Biotech) following instructions from the supplier.

Immunoblot and Antibodies:

Etoposide (20 μM) was treated in U20S cells (5×10⁵) for 12 hr to induce DNA damage. After ice-cold PBS wash (2×), cells were lysated in RIPA buffer. After centrifugation (12,000×g, 15 min) at 4° C., clear lysates were prepared with SDS-sample buffer is added then heat denatured for SDS-PAGE and immunoblots. The following primary antibodies were used.

| Antibody | Reference | Catalog number |
|---|---|---|
| FLAG | Sigma | F7425 |
| HA | Sigma | H6908 |
| CHK1 | Cell Signaling | #2360 |
| Phospho-CHK1 (S317) | Cell Signaling | #12302 |
| CHK2 | Cell Signaling | #6334 |
| HDM2 | Santa Cruz | #11973 |
| CDK12 | Cell Signaling | sc-7894 |
| CCNK | Abcam | Ab85854 |
| GAPDH | Cell Signaling | #5174 |
| p53 | Santa Cruz | sc-126 |
| 4E-BP1 | Cell Signaling | #9644 |
| Phospho-4E-BP1 (Thr37/46) | Cell Signaling | #2855 |
| Phospho-4E-BP1 (Ser65) | Cell Signaling | #9451 |
| Phospho-4E-BP1 (Thr70) | Cell Siganling | #9455 |
| CDK9 | Santa Cruz | sc-8338 |
| CCNT1 | Santa Cruz | sc-10750 |
| RNAPII Ser2-P | Bethyl | A300-654A |
| MTCO1 | Abcam | ab14705 |
| MTCO1 (COX1) | Santa Cruz | sc-23982 |
| MTCO2 (COX2) | Santa Cruz | sc-514489 |
| ND1 | Proteintech | 19703-1-AP |
| ATP5A1 | Proteintech | 14676-1-AP |
| CRIF1 | Santa Cruz | sc-374122 |
| VDAC1 | Santa Cruz | sc-390996 |
| Myc | Santa Cruz | sc-764 |
| NRF2 | R&D Systems | AF3925 |
| PAR/pADPr | R&D Systems | 4336-APC-050 |
| PARP1 | Santa Cruz | sc-7150 |
| CDK2 | Santa Cruz | sc-163 |
| CDK7 | Santa Cruz | sc-529 |
| CDK8 | Santa Cruz | sc-1521 |
| RNAPII Ser5-P | Bethyl | A304-408A |
| RNAPII Ser7-P | Cell Signaling | #13780 |
| MYCL | R&D Systems | AF4050 |
| MTSS1 | Cell Signaling | #43855 |
| MAP4K3 | Cell Signaling | #96135 |
| SMARCA4 (Brg1) | Cell Signaling | #3508 |
| SMARCB1 (SNF5) | Cell Signaling | #8745 |

Metabolic Labeling and De Novo Protein Synthesis Analysis:

Rapidly growing U2OS cells were incubated with methionine-free DMEM for 2 hr to deplete methionine. L-aziohomoalanine (Click-IT AHA; Thermo Fisher, C10102) was directly added to the media at the final concentration of 5 mM. The cells were incubated for 1 hr to incorporate AHA into newly synthesized proteins. For detection of newly synthesized Chk1 protein from the metabolic labeling, immunoprecipitation was carried out with Chk1 antibody (Santa Cruz, sc-8404) for 12 hr. After washing, immunoprecipitant was coupled with biotin-conjugates (biotin alkyne; Thermo Fisher, B10185) using Click-IT Protein Reaction Buffer Kit (Thermo Fisher C10276) according to the manufacturer's protocol. Immunoblot from SDS-PAGE analysis of total labeled protein and immunoprecpitant was carried out with NEUTRAVIDIN® Horseradish Peroxidase conjugate Kit (Thermo Fisher, A2664) according to the manufacturer's instruction.

Sucrose Density Gradient Assay:

To separate polysomes, RNA samples (0.5 ml) were loaded on the op of step-wise sucrose solutions (RNase-free 15%, 20%, 25% sucrose each 1 ml, and 30%, 35%, 40% each 0.5 ml). The polyallomer tubes was centrifuged in a swinging bucket SW 55 Ti (Beckman) at 40,000 rpm for 8 hours at 4° C. with an acceleration profile of 5 and deceleration profile of 5. Fractions were collected from the bottom of the tube and subjected for denature RNA agarose gel analysis or acid phenol/chloroform extraction followed qRT-PCR analysis.

siRNA Transfection:

The factor-specific siRNAs used in these studies were obtained from Life Technologies, and were transfected into cells using the LIPOFECTAMINE® RNAiMAX Transfection Reagent. Experimental analyses were performed 48 h after transfection. For negative control siRNA, the SILENCER® Negative Control #1 siRNA was used (Life Technologies). The siRNA information is listed below.

| GENE | Supplier | Cat # |
|---|---|---|
| CDK12 | Thermo Fisher | s28623 |
| CCNK | Thermo Fisher | s16800 |
| CDK9 | Thermo Fisher | s2835 |
| CCNT1 | Thermo Fisher | s2541 |
| CRIF1 | Thermo Fisher | s195549 (#1), s19551 (#2) |

Reverse Transcription and Quantitative RT-PCR:

Total RNA was isolated using the Trizol reagent (Life Technologies), and reverse transcription was performed using 3 μg of RNA extracted using SUPERSCRIPTIII® (Life Technologies) with oligo-dT. PCR measurements to cDNA were performed in triplicate using SYBR® Green master mixes (Life Technologies). Amplification was carried out in the ABI7300 (Applied Biosystems) with a 10 min DNA denaturation step at 95° C., followed by 40 cycles of 15 s at 95° C., 60 s at 60° C. The average of the technical replicates was normalized to GAPDH levels using the comparative CT method. Averages and standard deviation are the result of at least 3 independent experiments.

The qRT-PCR primers (SEQ ID NOs: 13-68) are shown below.

| GENE | FORWARD | REVERSE |
|---|---|---|
| GAPDH | TGTCATCAATGGAAATCCCATC (SEQ ID NO: 13) | AAAGTTGTCATGGATGACCTTG (SEQ ID NO: 41) |
| CDK12 | TACCGACCTCCAGAACTACTGC (SEQ ID NO: 14) | TAACATCAGGCCACACAGCTG (SEQ ID NO: 42) |
| CCNK | TCTCAAAGCTCCGAACCATCC (SEQ ID NO: 15) | CAACGGTGGATGAGTGGTCTC (SEQ ID NO: 43) |
| ATM | GAACTTTCAAGAACACTCAGCTCC (SEQ ID NO: 16) | GCTGGCATCCAACTTCTTGATC (SEQ ID NO: 44) |
| ATR | GGATGCCACTGCTTGTTATGAC (SEQ ID NO: 17) | GCTGCTTCCACTCTGTACGTG (SEQ ID NO: 45) |
| BRCA1 | TCTGGAATCAGCCTCTTCTCTG (SEQ ID NO: 18) | GTATCAGTAGTATGAGCAGCAGC (SEQ ID NO: 46) |
| FANCD2 | GTGCTCACTCGGTTAAAGCAC (SEQ ID NO: 19) | AGGATGTCTTGCTGCCATCTG (SEQ ID NO: 47) |
| FANC1 | GCTAAAGGAAACAGGGCATGTG (SEQ ID NO: 20) | CAAGCACAGCAGTGAGATGCTC (SEQ ID NO: 48) |
| CHK2 | GTTCAGCAAGAGAGGCAGAC (SEQ ID NO: 21) | GCTTCTTTCAGGCGTTTATTCC (SEQ ID NO: 49) |
| TP53 | TTGCGTGTGGAGTATTTGGATG (SEQ ID NO: 22) | AGTGTGATGATGGTGAGGATGG (SEQ ID NO: 50) |
| FOS | GTGAAGACCATGACAGGAGG (SEQ ID NO: 23) | TGTCTCCGCTTGGAGTGTATC (SEQ ID NO: 51) |
| CHK1 | ACTTCAGGTGGTGTGTCAGAG (SEQ ID NO: 24) | GCTGGTATCCCATAAGGAAAGA (SEQ ID NO: 52) |
| KLF17 | AGCGTGGTATGAGCTACTGC (SEQ ID NO: 25) | GCATCCTTAGATTCCCACCG (SEQ ID NO: 53) |
| SRP19 | TAAGAAGACCATCGCAGAGG (SEQ ID NO: 26) | CCTCTGTATTGGACATCACGATTC (SEQ ID NO: 54) |
| TMA7 | CGAAGGTGGCAAGAAGAAGC (SEQ ID NO: 27) | GATTTCTTAATTCCACCTGTGGC (SEQ ID NO: 55) |
| SLIRP | GCGCTGCGTAGAAGTATCAATC (SEQ ID NO: 28) | CTGAACCCAACCCAAACCTC (SEQ ID NO: 56) |
| RPL26 | CAGAAGTACAACGTGCGATCC (SEQ ID NO: 29) | CCTACGTGGACAGTTGTGCC (SEQ ID NO: 57) |
| CRIF1 | CTACGCGGCTAAGCAGTTCG (SEQ ID NO: 30) | GTACCATTCGCGTTCTTCGG (SEQ ID NO: 58) |
| YY2 | TACCAGGCATTGATCTCTCAG (SEQ ID NO: 31) | CCACATTCTGCACATACGTG (SEQ ID NO: 59) |
| PEG3 | ACCTCACTGAGCACCAGAAG (SEQ ID NO: 32) | GAGGTCTTCGCTGGTAGCAA (SEQ ID NO: 60) |
| FTH1 | GCTGAGAAACTGATGAAGCTGC (SEQ ID NO: 33) | CCAGTTTGTGCAGTTCCAGT (SEQ ID NO: 61) |
| RABGEF1 | ATCACGCGCTTCTGCAATCC (SEQ ID NO: 34) | TTGCTCCTGGGAGAGGTCT (SEQ ID NO: 62) |
| SBNO1 | CCAGCTAACAGTAACACCAAC (SEQ ID NO: 35) | CTCAGGGCCACCAAGTTCAT (SEQ ID NO: 63) |
| CISL | TACTGGTGCTCTTGAAGGAC (SEQ ID NO: 36) | GATTCCTCAGAGTCCAGGCC (SEQ ID NO: 64) |
| MAGEH1 | GCCAAGGAAGCTCTGGTCTG (SEQ ID NO: 37) | CATACTCCACCGGACTGCTA (SEQ ID NO: 65) |
| PFDN5 | GTCCCACTGACGAGTTCTATG (SEQ ID NO: 38) | CATGGCGTGCTTCTCCTGAAG (SEQ ID NO: 66) |
| PSMA4 | TACATTGGCTGGGATAAGCAC (SEQ ID NO: 39) | CAAGTGCTGACTTCAAGGTC (SEQ ID NO: 67) |
| ENY2 | CTGGAGAAAGAGAACGCCTCAAAG (SEQ ID NO: 40) | GGAGTGATTTCAGCCACCAA (SEQ ID NO: 68) |

Affinity Purification of FLAG-Tagged Protein Complexes and MudPIT Analysis:

Stable expression of HA-tagged full-length human CDK12 in HEK293 cells was established using G418 selection. Cells from fifteen 150 mm dishes were extracted using IP buffer (50 mM HEPES-NaOH pH 7.9, 300 mM NaCl, 1% NP-40, 10 mM MgCl₂, 15% glycerol) with protease inhibitors to a final volume of 15 ml, and homogenized by dounce. Extracts were clarified by centrifugation (14,000×g for 15 min at 4° C.). FLAG-M2 beads (Sigma) were incubated with the supernatant in a ratio of 80 μl slurry (50% beads in slurry) to 6 mg of total protein for 4 h on a roller at 4° C. The beads were washed in IP-wash buffer (25 mM HEPES-NaOH pH 7.9, 300 mM NaCl, 0.2% NP-40 four times for 3 min at 4° C. and then twice in FLAG-elution buffer (25 mM Tris-HCl, pH 7.5, 50 mM NaCl). Finally, the sample was incubated with FLAG peptide (200 μg/ml; Sigma) in elution buffer for 30 min at room temperature with rotation.

Determination of Protein Half-Life:

To measure the half-life of endogenous CHK1 and p53 proteins, U20S cells in a 6-well dish were transfected with siRNAs, as indicated in each figure. After 48 h, CHX (cycloheximide, 50 μg/ml at the final) was directly added to cells and incubated for the times indicated in each figure. Cells were washed twice with PBS and lysed in RIPA buffer (50 mM Tris-Cl, pH 7.7, 150 mM NaCl, 1 mM EDTA, 1% NP-40 (v/v), 0.1% SDS(w/v), 0.1% NaDeoxycholate (w/v) to prepare cell extracts. After centrifugation (14,000×g for 15 min at 4° C.), the soluble fraction was subject to SDS-PAGE and immunoblot analysis. The immunoblot signals were analyzed and quantified by ImageJ to obtain the half-life.

Chromatin Immunoprecipitation (ChIP) Experiments:

ChIP experiments were carried out as described in the previous report. Briefly, 5×10⁶ cells were twice washed with ice-cold PBS and serially cross-linked with 2 mM DSG (Disuccinimidylglutarate) for 20 min and 1% (v/v) formaldehyde for 10 min at room temperature. The cross-linking reaction was stopped by adding glycine to a final concentration of 0.125 M for 10 min at room temperature. Cells were washed twice with cold PBS. Cell pellets were lysed in 0.5 mL of Lysis buffer (50 mM Tris-HCl, pH 8.0, 10 mM EDTA, 1% SDS (v/v) supplemented with Protease Inhibitor Cocktail (Calbiochem). Chromatin was sonicated to generate DNA-fragments to an average size of 300-500 bp using a Model 505 Sonic Dismembrator (Fisher Scientific) with 5 cycles of 10 s/on and 60 s/off. After centrifugation at 13,000 g for 20 min, immunoprecipitation was performed using 40 μg of chromatin and 2-3 μg of antibody in ChIP buffer (20 mM Tris-HCl pH 8.0, 150 mM NaCl, 2 mM EDTA, and 1% Triton X-100) combined with the Protease Inhibitor Cocktail. After overnight incubation at 4° C., 60 μl slurry of protein A or G agarose was added for 1 hr with rotation. After stepwise wash with wash buffers, immune complexes were eluted in the buffer containing 1% SDS and 100 mM NaHCO₃. Crosslinking was reversed by heating at 65° C. for 4 hr with addition of proteinase K (Sigma). Eluted DNA and 10% of input chromatin were purified using phenol-chloroform extraction followed by isopropanol precipitation or using QIAQUICK® PCR purification (Qiagen), according to the manufacturer instructions. ChIP DNA is analyzed by SYBR® Green master mixes (Life Technologies) using described primer sets. qPCR is carried out in the ABI7300 (Applied Biosystems) with a 10 min DNA denaturation step at 95° C., followed by 40 cycles of 15 s at 95° C., 60 s at 60° C. PCR measurements were performed in triplicate. The average of the technical replicates was normalized to input DNA per set of primer using the comparative CT method. Following antibodies were used to precipitate chromatin and ChIP primers were used for qPCR of ChIP DNA.

| Antibody | Reference | Catalog number |
|---|---|---|
| CDK12 | Abcam | ab57311 |
| RNAPII Ser2-P | Cell Signaling | #13499 |
| eIF4E | Santa Cruz | sc-9976 |
| 4E-BP1 | Santa Cruz | sc-514073 |
| P-4E-BP1 (Thr 70) | Santa Cruz | sc-18092-R |

SEQ ID NOs: 69-80 are shown below:

| GENE | FORWARD | REVERSE |
|---|---|---|
| CHK1 + 85 | TGTCATCAATGGAAATCCCATC (SEQ ID NO: 69) | CACGCAGTCAAGTGTGTGTG (SEQ ID NO: 75) |
| CHK1 + 109 | AAGACCGGGCTGAAGTAAAGC (SEQ ID NO: 70) | AGGCTTCGAAAGAACTGGCC (SEQ ID NO: 76) |
| CHK1 + 3327 | GTAGATATGAAGCGTGCCGTAG (SEQ ID NO: 71) | GCTCTCCTCCACTACAGTAC (SEQ ID NO: 77) |
| CHK1 + 38506 | TGCAGGCTCAAGCAATCCTC (SEQ ID NO: 72) | CTGGGCAACATGGTGAGACC (SEQ ID NO: 78) |
| RPL26 | AGCCACTAGGTGACACTAGC (SEQ ID NO: 73) | CTACACGCTGCTTCCGGTTC (SEQ ID NO: 79) |
| TMA7 | CATGCCCTGCTGTCTACCTG (SEQ ID NO: 74) | GAGTGGCAGACGATTGGTCG (SEQ ID NO: 80) |

RNA Immunoprecipitation (RIP) Assay:

All steps were conducted at 4° C. in RNase-free conditions. U20S cells (5×10⁶) were transfected with the indicated siRNAs and washed twice with cold PBS. Cells were collected in 1.5 ml of RNA lysis buffer (25 mM Tris-HCl pH 7.5, 150 mM KCl, 5 mM EDTA, 1% NP-40 (v/v), and freshly added 0.5 mM DTT and 400 U/ml RNase inhibitor (NEB) and homogenized by dounce. After centrifugation at 13,000×g for 20 min, 0.7 ml of clear lysate was transferred to new tubes in addition with antisera (5 μg/IP) for immunoprecipitation. Antibody and lysate mixtures were incubated overnight with rotation. Aliquots of 60 μl of protein A or G (50% slurry) were added in the mixture and incubated for additional 2 hr. The agarose slurry was collected and washed three times in 1 ml of RNA Lysis buffer followed by centrifugation at 1,000×g for 2 min. After the final wash, 1 ml of TRIZOL® was used to extract captured RNAs from the immune complexes. After DNA digestion by RNase-free DNase I (NEB), RNA was precipitated by isopropyl alcohol or concentrated using the RNA Clean and Concentrator kit [IS THIS A TRADEMARK?] (Zymo Research). Reverse transcription and qRT-PCR for quantitative RNA analysis was carried out as described above.

| Antibody | Reference | Catalog number |
|---|---|---|
| eIF4E | Santa Cruz | sc-9976 |
| eIF4G | Santa Cruz | sc-11373 |
| control IgG | Santa Cruz | sc-2027 |

RNA-Seq and RIP-Seq Analysis:

High throughput sequencing was performed. For detail of analysis, sequenced reads were quality-tested using FASTQC and aligned to the hg19 human genome using the STAR aligner version 2.4.0 k (Dobin et al., Bioinformatics. 2013; 29(1):15-21. doi: 10.1093/bioinformatics/bts635. pmid:23104886). Mapping was carried out using default parameters (up to 10 mismatches per read, and up to 9 multi-mapping locations per read). The genome index was constructed using the gene annotation supplied with the hg19 Illumina iGenomes collection and overhang value of 100. Raw gene expression was quantified across all gene exons (RNA-Seq) or across gene bodies (RIP-Seq), using the top-expressed isoform as proxy for gene expression, and differential gene expression was carried out using the edgeR package version 3.6.8 (Robinson et al. (2010) Bioinformatics, 26, pp. 1) using duplicates to compute within-group dispersion. Differentially expressed genes were defined as having a false discovery rate (FDR)<0.05 and a log 2 fold change >0.5 when comparing two experimental conditions. Genes showing mTOR-specific binding and CDK12-specific binding were defined as those significantly downregulated in the Rapamycin RIP or siCDK12 RIP compared to the control RIP, while genes that showed mTOR-specific or CDK12-specific transcription were defined as those significantly differential between control and Rapamycin or siCDK12 total mRNA-Seq conditions.

Annotation and Enrichment Analyses:

GO term and KEGG pathway enrichment analysis, motif enrichment analysis, and genome annotation was carried out on gene sets using the HOMER analysis package (Heinz et al. (2010) Mol Cell 38: 576-589) and the Benjamini and Yekutieli general correction for multiple testing (Benjamini and Hochberg (1995) Journal of the Royal Statistical Society, Series B 57 (1): 289-300). Specifically, pathway enrichment analysis was carried out by checking for significant overlap in KEGG, Wikipathways, Reactome, and GO Biological Process databases. Motif enrichment analysis was carried out for the region −50 to 150 bp around gene TSS, searching for motif lengths of 8, 10, and 12, or using a set of all vertebrate motifs known to HOMER.

### Example 2

### CDK12 Regulates Gene-Specific Translation of the DNA Damage Response Kinase, CHK1

It was determined whether CDK12 is required for the activation of p53 target genes in response to DNA damage. Surprisingly, p53 could not be induced in response to DNA damage (etoposide treatment) in U20S bone osteosarcoma epithelial cells depleted of either CDK12 or CCNK (FIG. 1A). The failure to induce p53 was accompanied by a dramatic decline in steady-state levels of CHK1 and the catalytically active S317-phosphorylated CHK1 proteins, which phosphorylates and stabilizes p53 upon DNA damage (FIG. 1B). The loss of CHK1 in these cells was readily observed even in the absence of DNA damage. Expression of both CDK12 and CHK1 was greatly impaired in cells treated with CCNK siRNA, which indicates that each subunit is required for the stability of the CDK12 complex. In contrast, levels of the related CHK2 kinase and the HDM2 E3 ligase, which controls p53 stability, were unaffected by depletion of CDK12 or CCNK. These results indicate that CHK1, but not CHK2, is regulated by CDK12. Identical results were obtained in U20S cells treated with a different genotoxic agent, hydroxyurea (HU) (FIG. 1C). As expected, siRNA-mediated depletion of CHK1 was sufficient to prevent the induction of p53 by DNA damage in U20S cells (FIG. 8B). Protein half-life measurements further confirmed that p53 stability was unaffected in response to DNA damage in CDK12-knockdown cells (FIG. 8C). Unlike typical CDK12 target genes such as BRCA1, steady state levels of CHK1 mRNA were unaffected at reduced levels of CDK12 or CCNK1 (FIGS. 1D and 8A). It was concluded that CDK12:CCNK is required for the expression of CHK1, and for activation of p53 by DNA damage, but acts through an unexpected mechanism that does not involve transcription or mRNA processing.

Further investigation revealed that CDK12 depletion had no effect on CHK1 protein stability (FIGS. 1E and 8D), nor did it affect 3′-end cleavage or transport of CHK1 mRNA to the cytoplasm (FIGS. 8E and 8F). However, knockdown of CDK12 profoundly reduced de novo protein synthesis of CHK1, as determined by pulse-chase metabolic labeling experiments (FIG. 1F). This effect was highly selective for CHK1 because global protein synthesis was unaffected by the reduction of CDK12 or CCNK expression. These findings indicated a role for CDK12 in translation of CHK1 mRNA. To examine this possibility, the binding of CHK1 mRNA to ribosomes following polysome fractionation was analyzed on sucrose gradients. As shown in FIG. 1G, the sedimentation pattern of CHK1 mRNA was greatly affected in the CDK12-knockdown cells, indicating that CDK12 is required for efficient association of CHK1 mRNA with polysomes. In contrast, CDK12 knockdown had no effect on GAPDH mRNA distribution on polysomes, nor did it affect steady-state levels of GAPDH protein (FIG. 1A). These data indicated that CDK12 functions selectively to facilitate ribosome loading of CHK1 mRNA, and that the subsequent loss of CHK1 protein in CDK12-depleted cells prevents p53 induction in response to different types of DNA damage.

### Example 3

### CDK12 Regulates Translation and Binding of eIF4G to a Subset of mRNAs

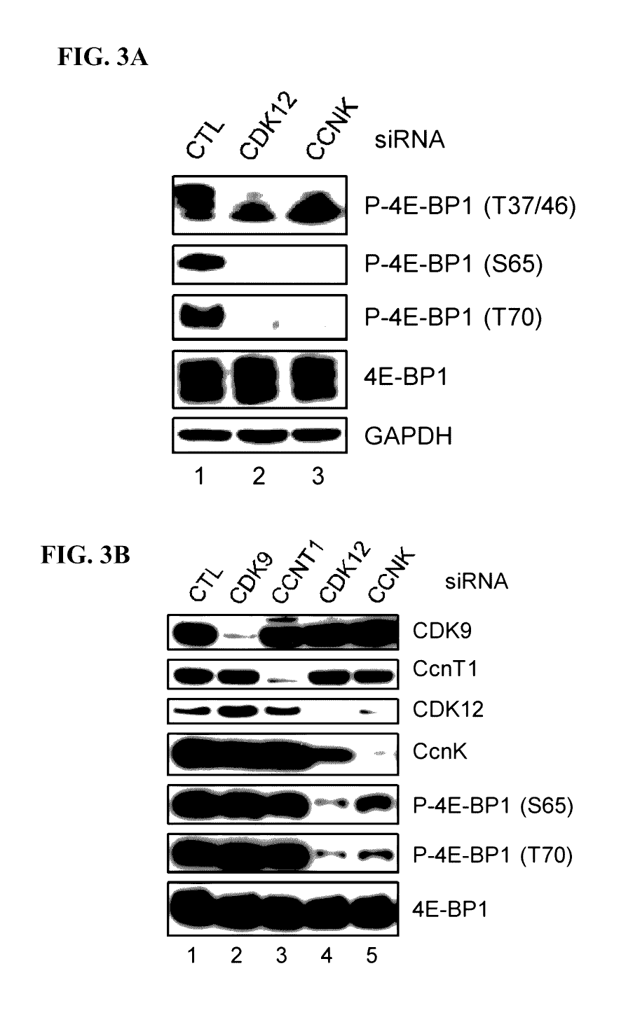

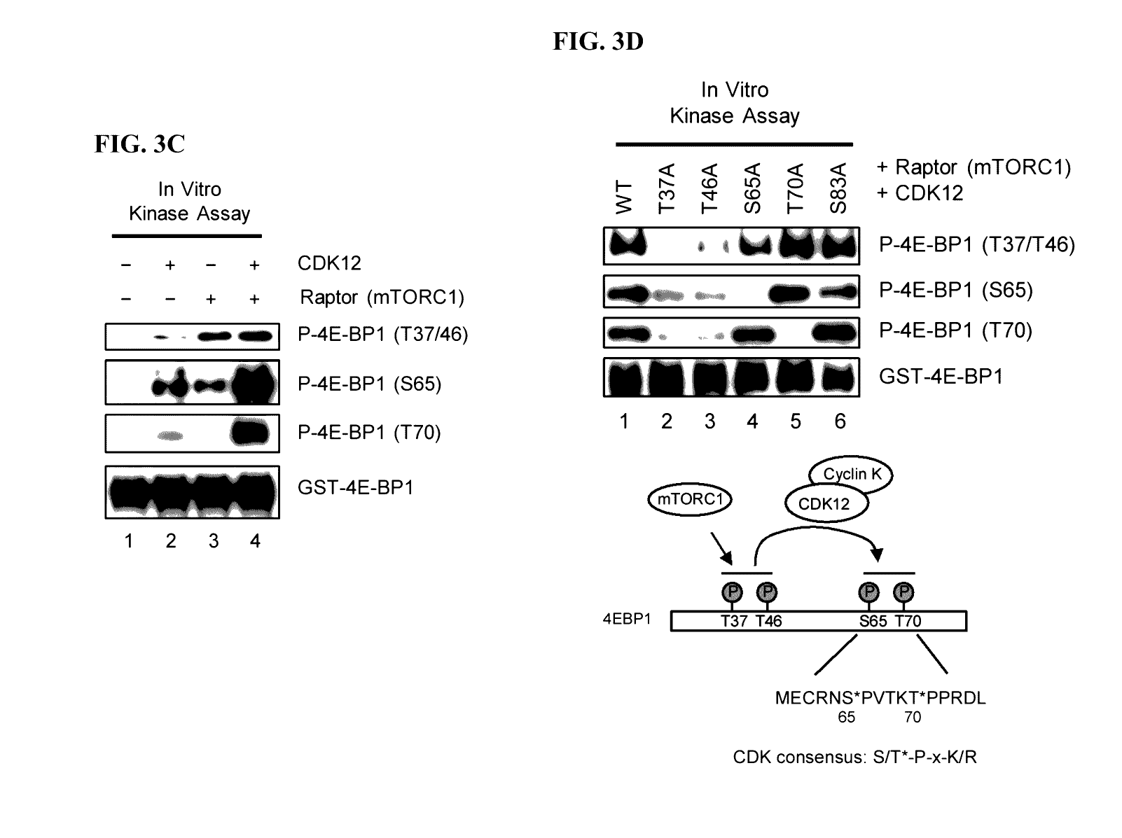

The loading of ribosomes onto mRNAs often requires the release of a translational repressor, 4E-BP1, which binds to eIF4E at the 5′-end mRNA cap. The release of 4E-BP1 depends on phosphorylation by the mTORC1 kinase, which then enables binding of eIF4G and translation of cap-dependent mRNAs (FIG. 2A, left panel). It was determined that CDK12 knockdown does not affect steady-state levels of eIF4E, eFI4G, or 4E-BP1 (FIGS. 9A and 3A). Consequently, RNA immunoprecipitation (RIP) experiments were carried out to assess whether CDK12 selectively facilitates binding of eIF4G to CHK1 mRNA. As shown in FIG. 2A (right panel), CDK12 depletion did not affect the association of the cap-binding protein eIF4E with CHK1 mRNA, indicating that CDK12 does not affect capping of CHK1 mRNA. In contrast, binding of eIF4G to CHK1 mRNA to eIF4G was significantly impaired. Consistent with the specific effects of CDK12 on CHK1 expression, CDK12 had no effect on binding of eIF4G or eIF4E to GAPDH mRNA. It was concluded that CDK12 discriminately controls the association of eIF4G with CHK1, but not GAPDH, mRNAs.

To identify other mRNAs that can be translated in a CDK12-dependent manner, high-throughput RNA sequencing (RNA-seq) analysis were performed of the eIF4G-bound mRNAs (RIP-seq) isolated from U2OS cells that express normal or reduced levels of CDK12 (FIG. 2B). In parallel studies, an RIP-seq analysis was carried out of eIF4G-bound mRNAs isolated from U2OS cells treated with the mTORC1 inhibitor Rapamycin, which blocks cap-dependent translation (FIG. 2C). To distinguish between transcription and translation targets of CDK12, total RNA-seq experiments were also carried out in control and CDK12-knockdown cells (FIG. 9B). Genes that were affected transcriptionally by CDK12 (1>log 2Δ; FIGS. 9B and 9C) were then excluded, in order to focus on primary translation targets. Collectively, this approach identified 1,001 mRNAs, including the top fifty hits listed in FIG. 2B, that bind to eIF4G in a CDK12-dependent manner. Similarly, this approach identified 2,961 rapamycin-sensitive mRNAs that require mTORC1 to efficiently bind eIF4G. The top fifty rapamycin/mTORC1-dependent mRNAs are listed in FIG. 9D. Group ontology analysis revealed that CDK12 affects the translation of many genes required for translation and RNA processing (FIG. 9F). Approximately 391 (39%) of the 1,001 CDK12-dependent mRNAs were also found to be rapamycin-sensitive, including 32 of the top 50 CDK12-dependent mRNAs (FIGS. 2B and 2D). Similarly, 29 of the top 50 rapamycin-sensitive genes were also sensitive to CDK12 (FIGS. 2D and 9D). Thus, translation of a specific subset of mRNAs very strongly depends on both CDK12 and mTORC1. Further analysis of the RIP-seq data revealed two other groups of mRNAs that are dependent on either CDK12 or mTORC1, but not both (FIG. 9E). For these mRNAs, CDK12 or mTORC1 presumably act in concert with other 4E-BP1 kinases (Qin et al., Cell Cycle 15, 781-786, 2016). The RIP-seq results were verified by normalized qRT-PCR for a subset of individual genes representative of each of the three categories (FIG. 2E), and validate the genome-wide results. These results indicate d that CDK12 controls eIF4G binding and translation initiation for a select subset of mRNAs, including CHK1 and many translation and RNA processing factors.

### Example 4

### CDK12 Directly Phosphorylates 4E-BP1 at S65 and T70

Previous studies demonstrated that release of the 4E-BP1 translation repressor from target mRNAs requires phosphorylation at multiple sites, including two sites (T37 and T46) that are phosphorylated by mTORC1, and two Ser/Thr-Pro phosphorylation sites (S65 and T70), which have been linked to various other kinases (Qin et al., Cell Cycle 15, 781-786, 2016). The observation that CDK12 regulates the stable association of a subset of mRNAs with eIF4G indicated that it might directly affect the phosphorylation and release of 4E-BP1 at target mRNAs. To assess this possibility, the phosphorylation status of total 4E-BP1 was analyzed by immunoblot analysis using phospho-specific antisera in CDK12- or CCNK-knockdown cells. As shown in FIG. 3A, global phosphorylation of 4E-BP1 at S65 and T70 was greatly impaired in cells depleted of CDK12 or CCNK. Moreover, reduced levels of CDK12:CCNK increased the migration of bulk 4E-BP1, consistent with a significant loss of overall phosphorylation, but did not affect the mTORC1-specific (T37, T46) phosphorylation sites. To assess whether 4E-BP1 phosphorylation is specific to CDK12, it was determined whether it might also be phosphorylated by the highly-related CDK9:CCNT1 (P-TEFb) complex. Knockdown of P-TEFb subunits had no effect on 4E-BP1 phosphorylation levels in vivo (FIG. 3B), indicating that this phosphorylation is CDK12-specific.

It was next addressed whether CDK12 can directly phosphorylate purified recombinant 4E-BP1 in vitro, alone or in combination with the mTORC1 kinase complex, which was immunopurified through the Raptor subunit. Previous studies indicated that 4E-BP1 phosphorylation at T37, T46 by mTORC1 primes subsequent phosphorylation at the Ser/Pro S65 and T70 sites. As shown in FIG. 3C, affinity-purified CDK12 weakly phosphorylated the 4E-BP1 substrate at all tested sites, whereas mTORC1 robustly phosphorylated 4E-BP1 at T37 and T46, but only weakly at S65, and not at T70. Overall, the combination of CDK12 and mTORC1 led to high levels of S65 and T70 phosphorylation of 4E-BP1, whereas phosphorylation at the T37 and T46 sites remained unchanged from the levels seen in reactions containing mTORC1 alone. To test whether phosphorylation at T37 and T46 potentiates phosphorylation at the Ser/Thr-Pro S65 and T70 sites in vitro, a series of point mutations in the 4E-BP1 substrate was analyzed in reactions containing both CDK12 and mTORC/Raptor kinases (FIG. 3D). Consistent with the observed cooperativity between these two kinases, point mutation of either T37 or T46 both eliminated phosphorylation by mTORC1/Raptor and strikingly reduced CDK12-mediated phosphorylation at S65 and T70 in vitro (FIG. 3D, lanes 2 and 3). These data indicate that CDK12-mediated phosphorylation of 4E-BP1 at S65 and T70 is enhanced by prior phosphorylation at T37 and T46 by mTORC1/Raptor. Thus, CDK12:CCNK directly phosphorylates 4E-BP1 at S65 and T70, especially when primed by prior phosphorylation by mTORC1 at T37, T46, and that 4E-BP1 is a major substrate for CDK12:CCNK, but not CDK9/P-TEFb, in vivo and in vitro (FIG. 3D, bottom).

### Example 5

### CDK12 Controls Binding of 4E-BP1 to Nascent mRNAs at CHK1 and Other Target Gene Promoters In Vivo

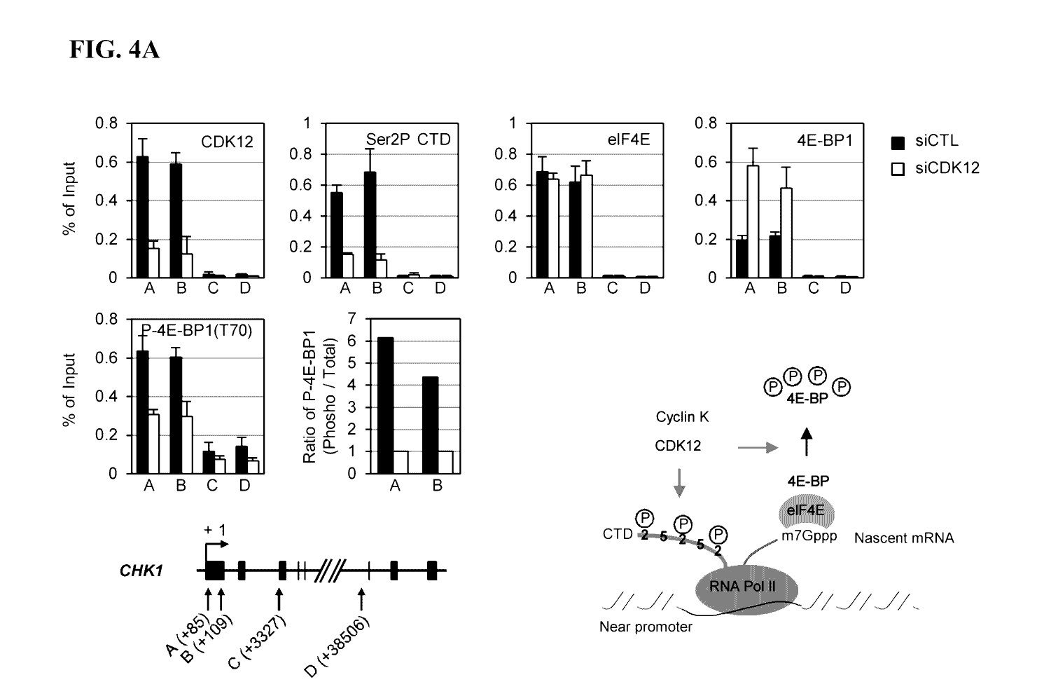

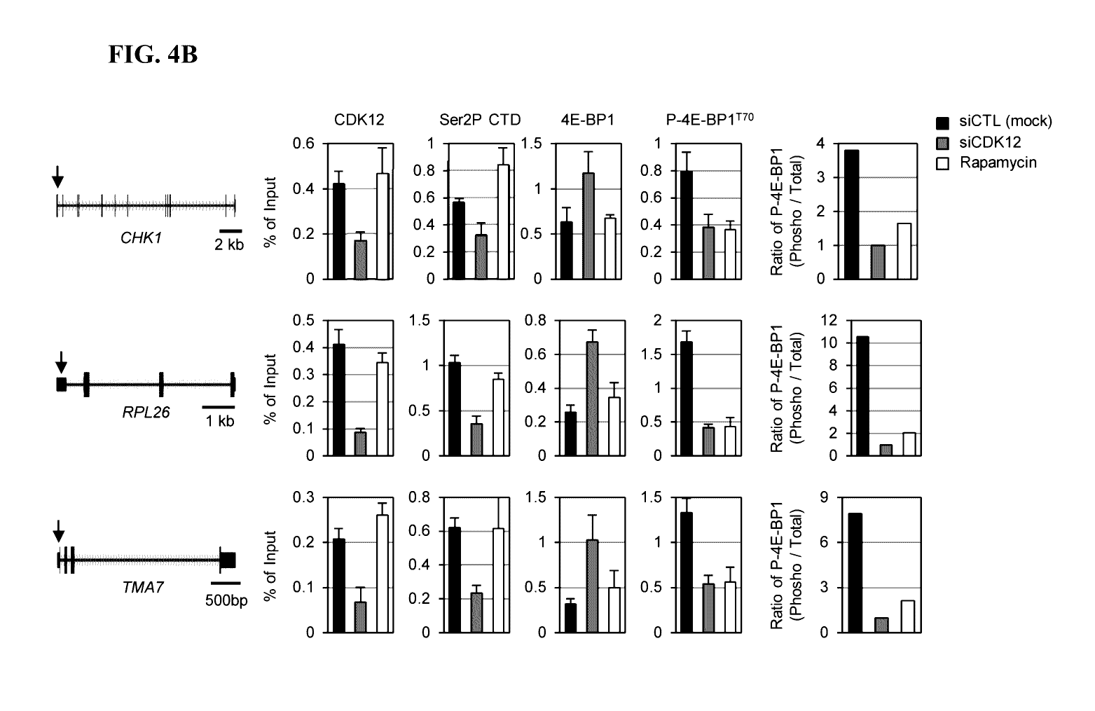

Because CDK12 can travel with RNAPII at target genes (Hsin et al., Genes Dev 26, 2119-2137, 2012), it was next determined whether it is present at the active CHK1 gene in U20S cells. Chromatin immunoprecipitation (ChIP) analysis indicated that CDK12 and Ser2P-RNAPII were both present at the CHK1 promoter in U20S cells (FIG. 4A). Knockdown of CDK12 reduced its level at the gene, and also lowered RNAPII CTD-Ser2P levels at the CHK1 promoter. Because eIF4E and 4E-BP1 have been detected in the nucleus (Rong et al., RNA 14, 1318-1327, 2008), it was questioned whether the eIF4E:4E-BP1 complex might be bound to nascent mRNA at the CHK1 gene promoter. Intriguingly, both 4E-BP1 and eIF4E were detected specifically by ChIP at the CHK1 promoter (FIG. 4A). Notably, depletion of CDK12 increased 4E-BP1 occupancy at the CHK1 promoter, whereas levels of eIF4E were unaffected. To assess whether CDK12 might phosphorylate 4E-BP1 to release it from CHK1 mRNA at the gene, the levels of T70-phosphorylated 4E-BP1 was examined. In contrast with total level of 4E-BP1, the amount of T70-phosphorylated 4E-BP1 at the CHK1 promoter decreased in CDK12-knockdown cells, indicating that reduced CDK12 phosphorylation of 4E-BP1 causes it to accumulate at the gene promoter.

To test whether the eIF4E:4E-BP1 complex detected at the CHK1 gene is bound to nascent mRNA, the ChIP experiments were repeated in the presence of α-amanitin, an inhibitor of RNAPII transcription. Consistent with this possibility, α-amanitin treatment largely abolished the association of both eIF4E and 4E-BP1 at the promoter, without affecting the occupancy of RNAPII or CDK12 (FIG. 10A). To test whether promoter-localized RNAPII-Ser2P and CDK12 might be a conserved feature of CDK12-regulated translation targets, ChIP-seq experiments were performed in U2OS cells using Ser2P and CDK12 specific anti-sera. Although the CDK12 ChIP-seq signal was limited by the relatively weak antibody, five genes containing high levels of both CDK12 and RNAPII CTD-Ser2P at the promoter region were selected (FIG. 10B) for further analysis. Interestingly, RNA immunoprecipitation revealed that CDK12 was required for binding of eIF4G to each of these five mRNAs (FIG. 10C). Moreover, the expression of each of these genes at the protein level was significantly reduced in CDK12 knockdown cells (FIG. 10D). Similar to the ChIP results at the CHK1 promoter, occupancy of 4E-BP1, but not eIF4E, at the RPL26 and TMA7 genes was increased in CDK12-depleted genes (FIG. 4B). In particular, it was noted that both RNAPII-Ser2P and levels of 4E-BP1 and T70P-4E-BP1 were sensitive to CDK12 levels. In contrast, rapamycin treatment affected 4E-BP1 phosphorylation and occupancy, without affecting RNAPII-Ser2P levels. These data indicate that CDK12 can control the release of 4E-BP1 from nascent mRNAs co-transcriptionally to mark target mRNAs for efficient translation in the cytoplasm.

### Example 6

### CRIF1 is a Potent and Selective Inhibitor of CDK12 Kinase Activity

The yeast homolog of CDK12, CTDK-I, is a ternary complex, consisting of the cyclin-kinase pair and a third subunit, CTK3, which stabilizes the complex (Hautbergue and Goguel, J Biol Chem 276, 8005-8013, 2001). To evaluate whether the human CDK12:CCNK complex might also contain unknown regulatory subunits, the CDK12 complex was affinity purified from HEK293 cells engineered to stably express the HA-tagged full-length CDK12. SDS-PAGE followed by silver stain analysis of the affinity-purified CDK12 complex revealed the presence of several unidentified protein bands, in addition to CDK12 and CCNK (FIG. 5A). MudPIT proteomics analysis of the CDK12 fraction revealed that several of these are heat shock chaperone proteins known to facilitate the assembly of CDK complexes (FIG. 5B). Most prevalent among the unidentified interacting proteins was CRIF1 (GADD45GIP1), which had a very high coverage rate (30.2%) in the MudPIT analysis. Co-immunoprecipitation analysis confirmed that high levels of endogenous CRIF1 are present in native CDK12 complexes isolated from HEK293 cells (FIG. 5C). Moreover, CRIF1 was not present in CDK2, CDK7, CDK8 or CDK9 complexes (FIGS. 5D and 11A), indicating that the association of CRIF1 with CDK12 is highly selective.

### Example 7

### CRIF1 Binds to a Regulatory Loop Adjacent to the CDK12 Kinase Domain

To determine the role of CRIF1 within the CDK12 complex, a series of truncated mutants were generated to map the domains that mediate the interaction between these two proteins. Analysis of ectopically expressed HA-CDK12 truncation mutants in HEK293 cells revealed that the C-terminal region of CDK12 (aa985-1490) is critical for binding to full-length FLAG-CRIF1 protein (FIG. 11B). The N-terminal domain of CDK12 (aal-787) mediates binding to CCNK (Bösken et al., Nat Commun 5, 3505, 2015). Further analysis with additional mutants revealed that a C-terminal regulatory loop adjacent to the CDK12 kinase domain (aa985-1113) is critical for binding to CRIF1 in vivo (FIG. 5E).

It was next evaluated how CRIF1 targets CDK12. Deletion of the C-terminal end (aal-183) of CRIF1 destroyed binding to a CDK12 fragment (aa985-1490; FIG. 5F, upper panel). This region of CRIF1 contains a motif conserved with the yeast CTK3 subunit of the CTDK-I complex (FIG. 5F, bottom panel), indicating that this region may be functionally conserved among CDK12/CTK1-interacting proteins.

### Example 8

### CRIF1, but not CDK12, Controls Mitochondrial MTCO-1 Expression

The association of CRIF1 with CDK12 was unexpected because it is known to reside in both the nucleus and mitochondria, where it associates with mitoribosomes and facilitates the synthesis and insertion of nascent OXPHOS proteins into the mitochondrial membrane (Chung et al., J Biol Chem 278, 28079-28088, 2003; Kim et al., Cell Metab 16, 274-283, 2012; Ryu et al., PLoS Genet 9, e1003356, 2013; Nagar et al., PLoS One 9, e98670, 2014; Ran et al., PLoS One 9, e85328, 2014). To assess whether CDK12, like CRIF1, is important for mitochondrial function, the effects of CDK12 or CRIF1 knockdown was evaluated on cell growth, expression of mitochondrial OXPHOS proteins, and the sub-cellular localization of each factor. Growth of cancer cells in high glucose under typical cell culture conditions produces ATP through glycolysis and lactic acid fermentation, rather than oxidation of pyruvate in mitochondria, but the latter become essential for growth on galactose. Consequently, the effects of CRIF1 or CDK12 knockdown was first analyzed on U2OS cell growth in each medium.

As shown in FIG. 5G, CRIF1 depletion had no effect on cell growth in glucose, but led to massive apoptosis in cells grown in galactose. In contrast, lowered CDK12 or CCNK levels (FIG. 11C) had no differential effect on growth on these media, indicating that they are not essential for switching between nutrient growth conditions. Moreover, knockdown of CRIF1, but not CDK12 or CCNK, potently and specifically inhibited the expression of the key mitochondrial OXPHOS protein MTCO-1 (FIG. 5H). Reproducibly, ablation of CDK12 or CCNK reduced global RNAPII-Ser2P levels, whereas depletion of CRIF1 had the opposite effect and markedly increased RNAPII-Ser2 phosphorylation. These data indicate that CRIF1 can inhibit global CDK12 CTD kinase activity in vivo, and that CRIF1, but not CDK12, is critical for MTCO-1 expression in the mitochondria and oxidative phosphorylation.

The subcellular localization of CDK12 and CRIF1 was assessed in fractions from U2OS cells. As shown in FIG. 5I, CRIF1 was detected mostly in the mitochondria-containing organelle fraction (M) in U2OS cells grown in high glucose, whereas CDK12 was predominantly nuclear. However, approximately 20-30% of native CDK12 could be detected in the organelle (mitochondrial) and cytoplasmic fractions (FIG. 11D). Knockdown of CRIF1 reduced the level of CDK12 in the organelle fraction, indicating it may be transported by CRIF1 to the mitochondria. These data indicate that CRIF1 inhibits CDK12 kinase activity and can sequester it in the mitochondria in vivo.

### Example 9

### CRIF1 is a Potent and Selective Inhibitor of CDK12 In Vivo and In Vitro

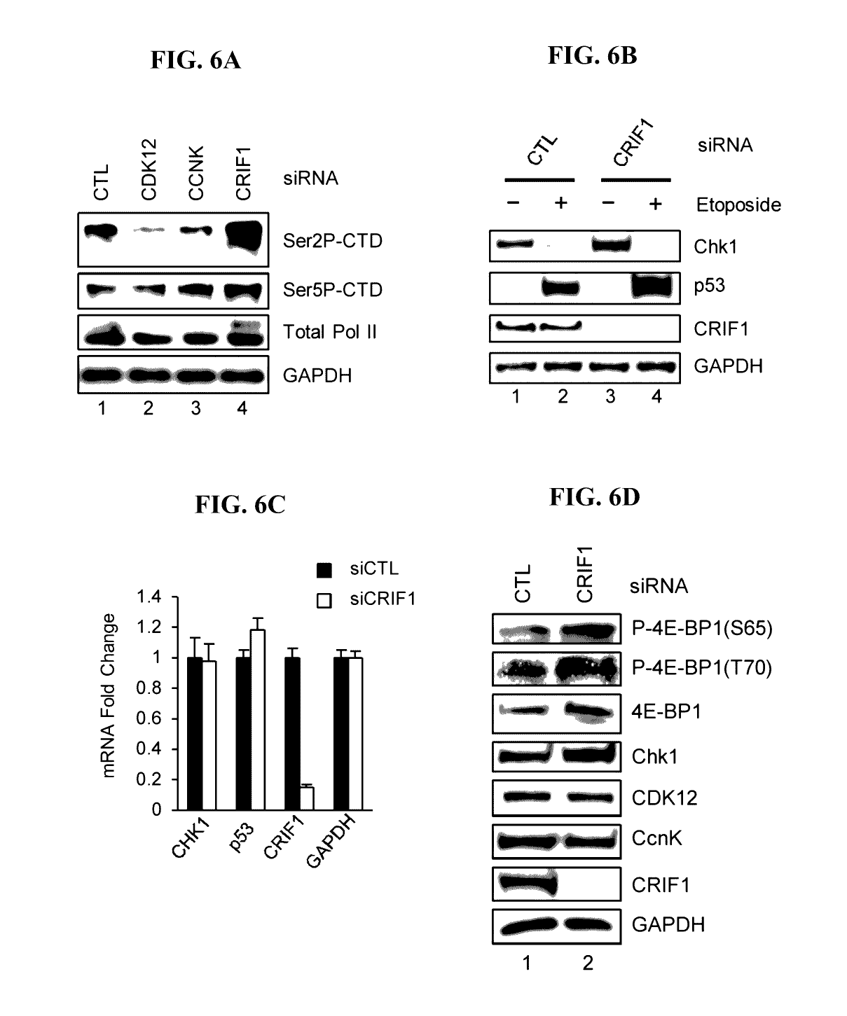

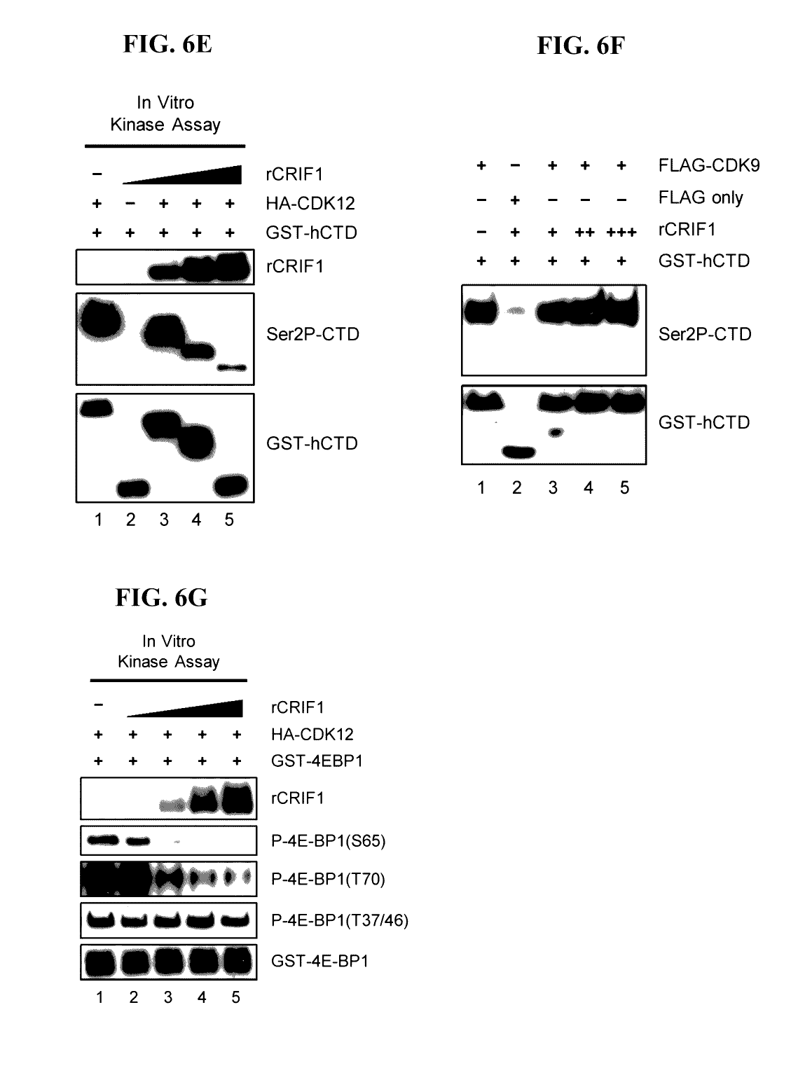

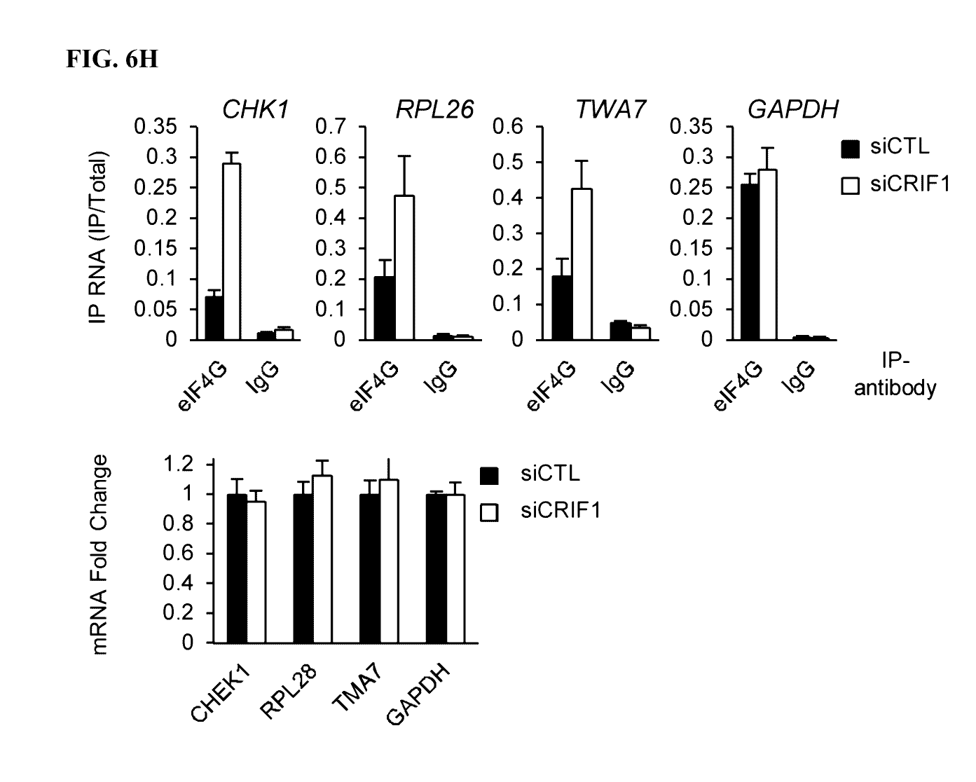

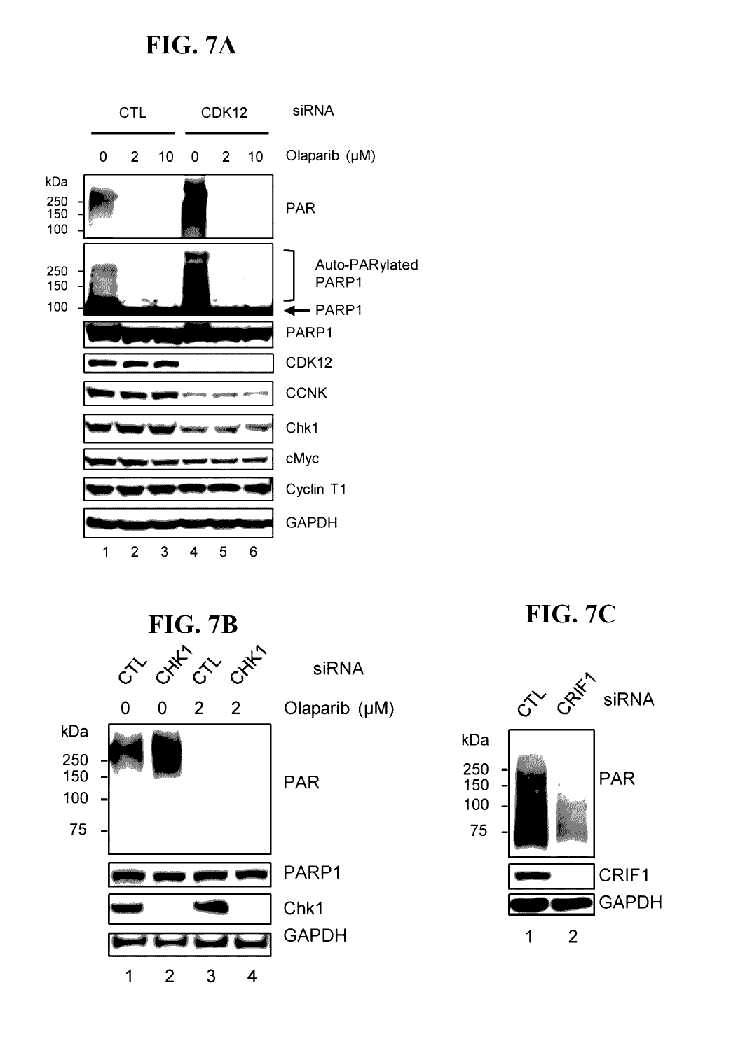

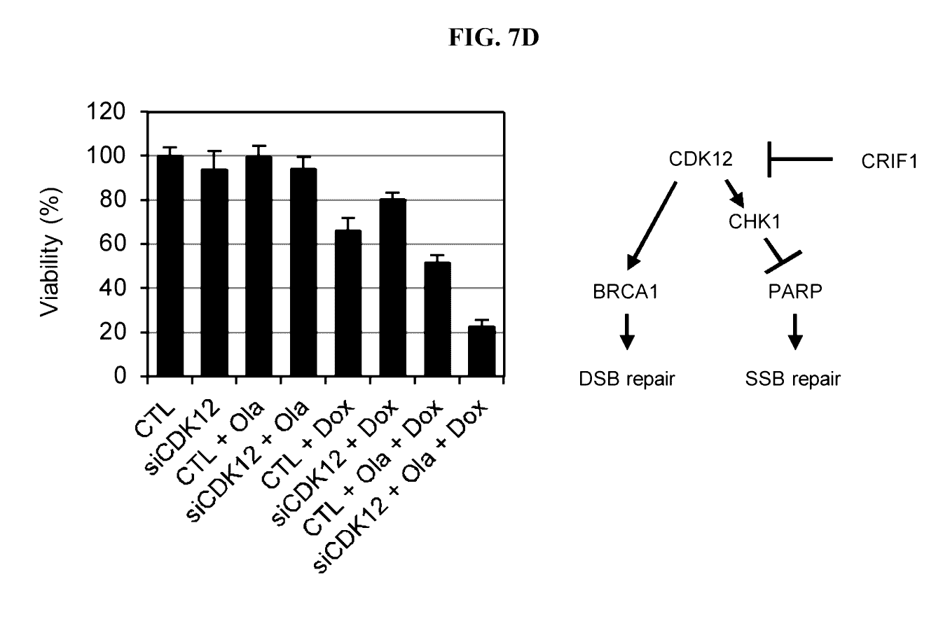

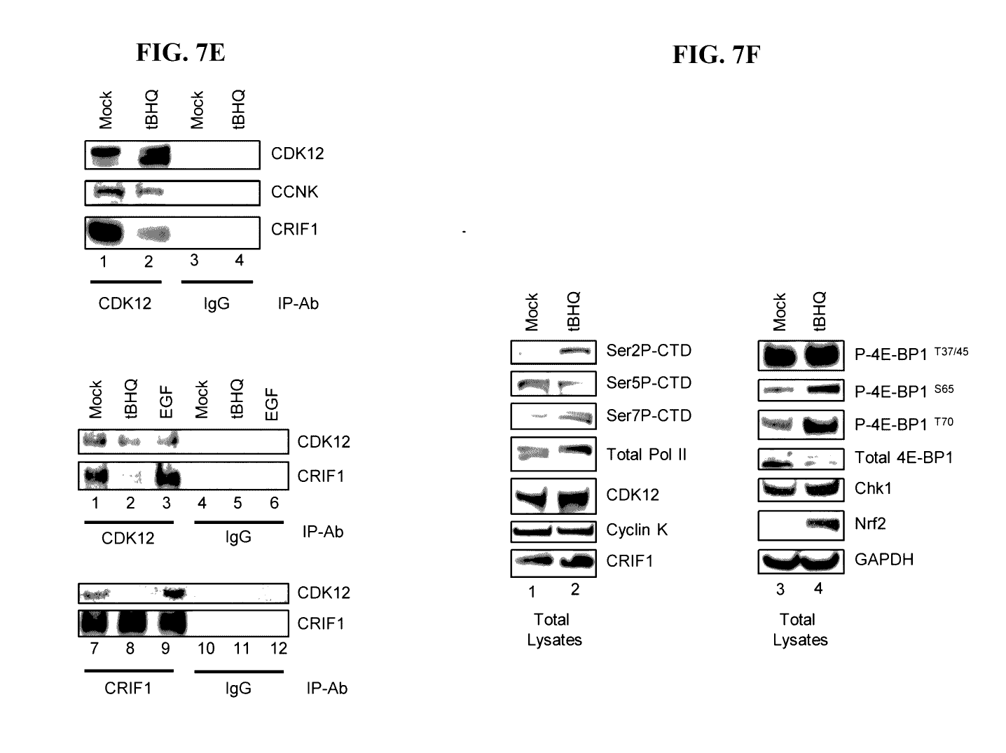

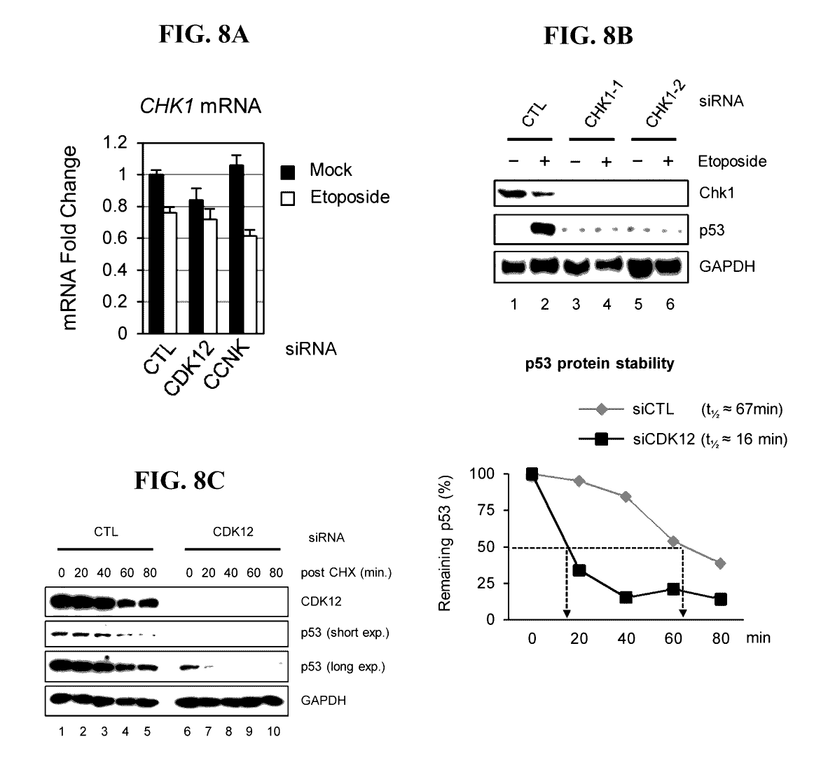

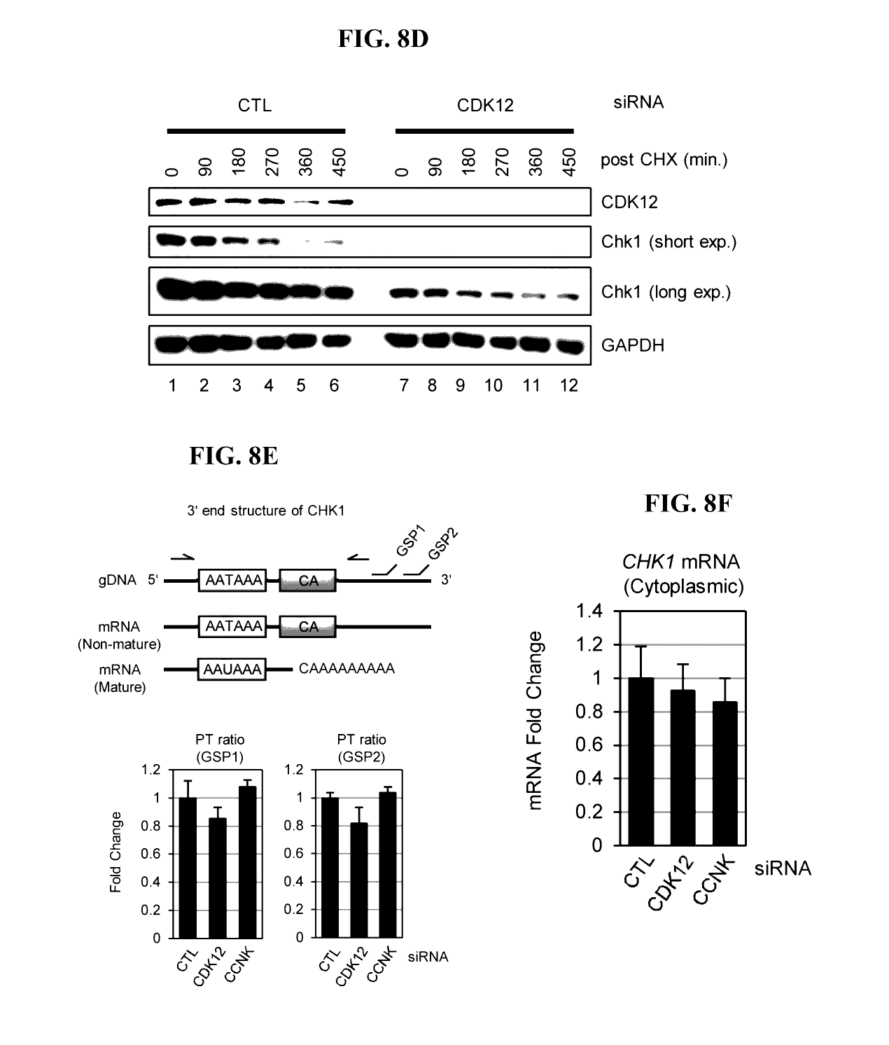

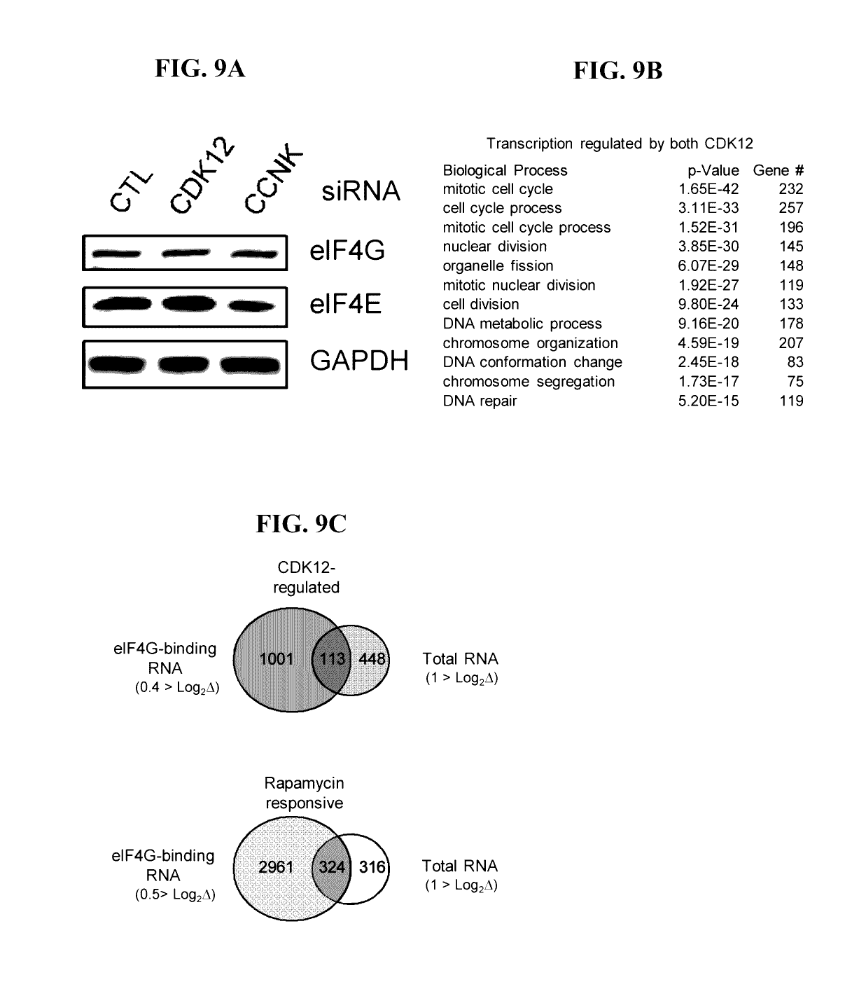

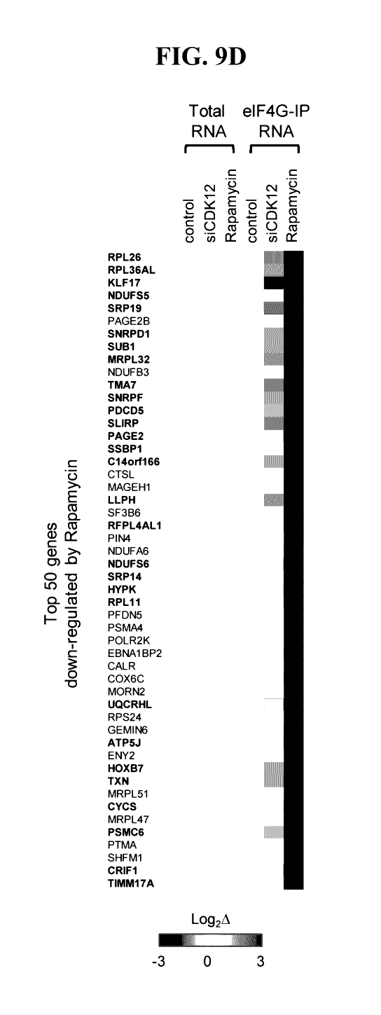

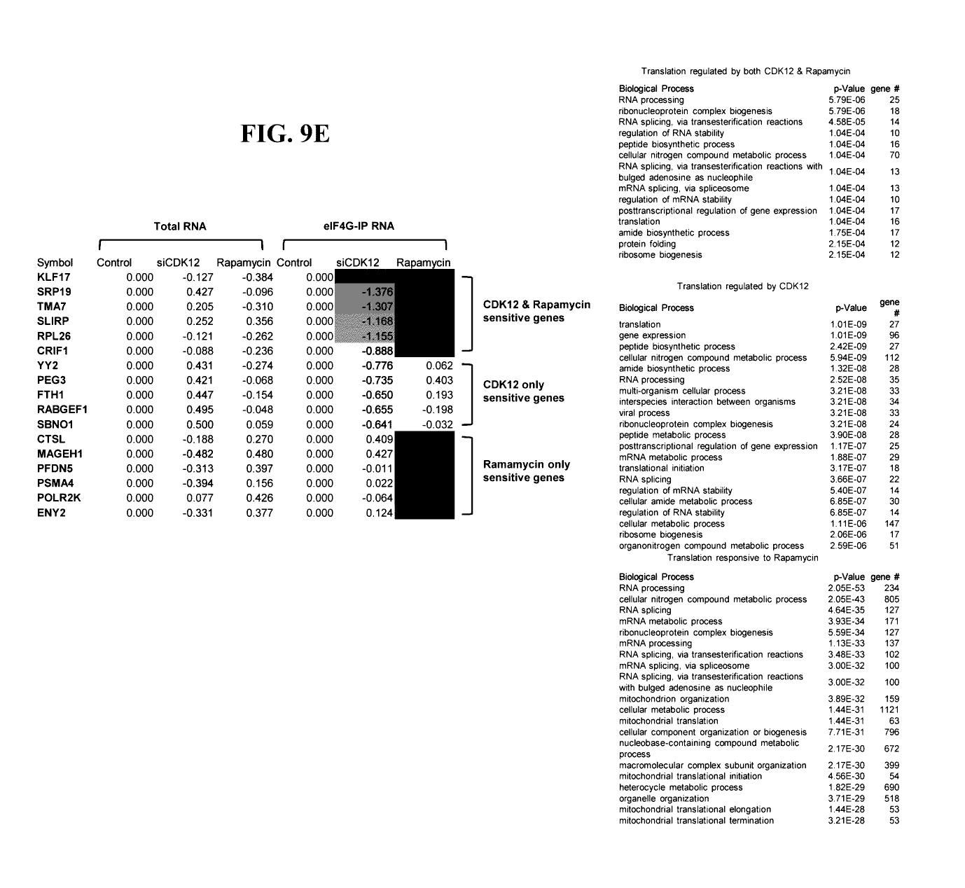

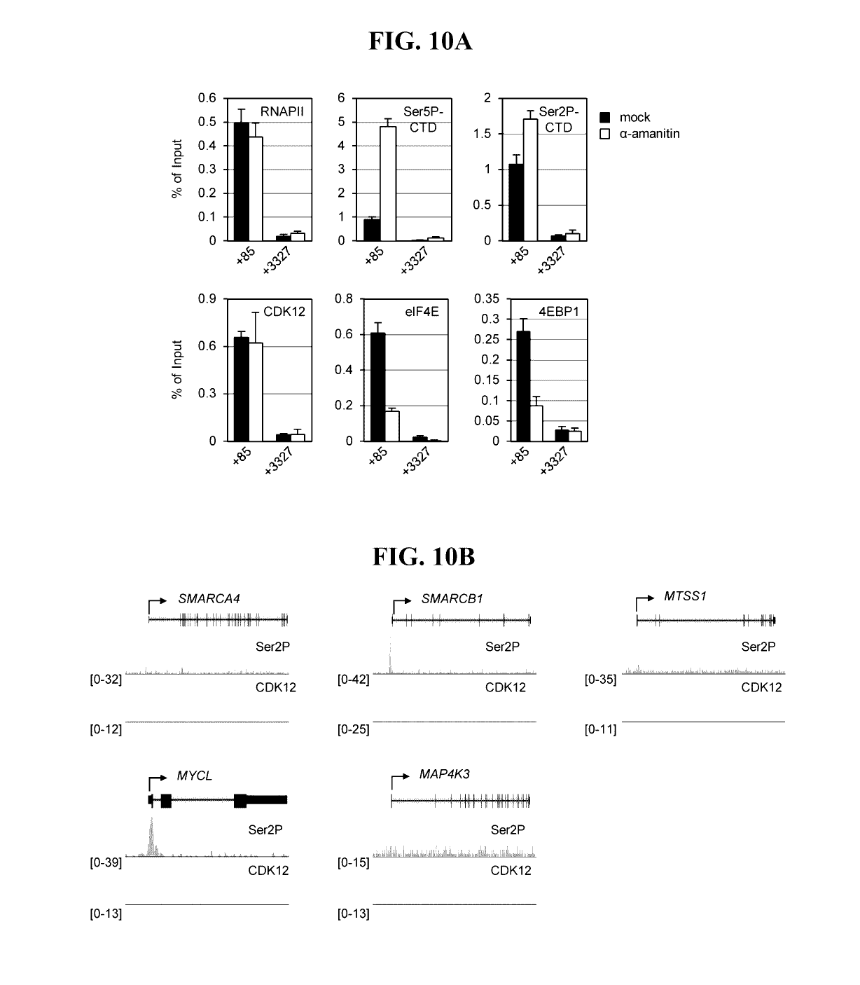

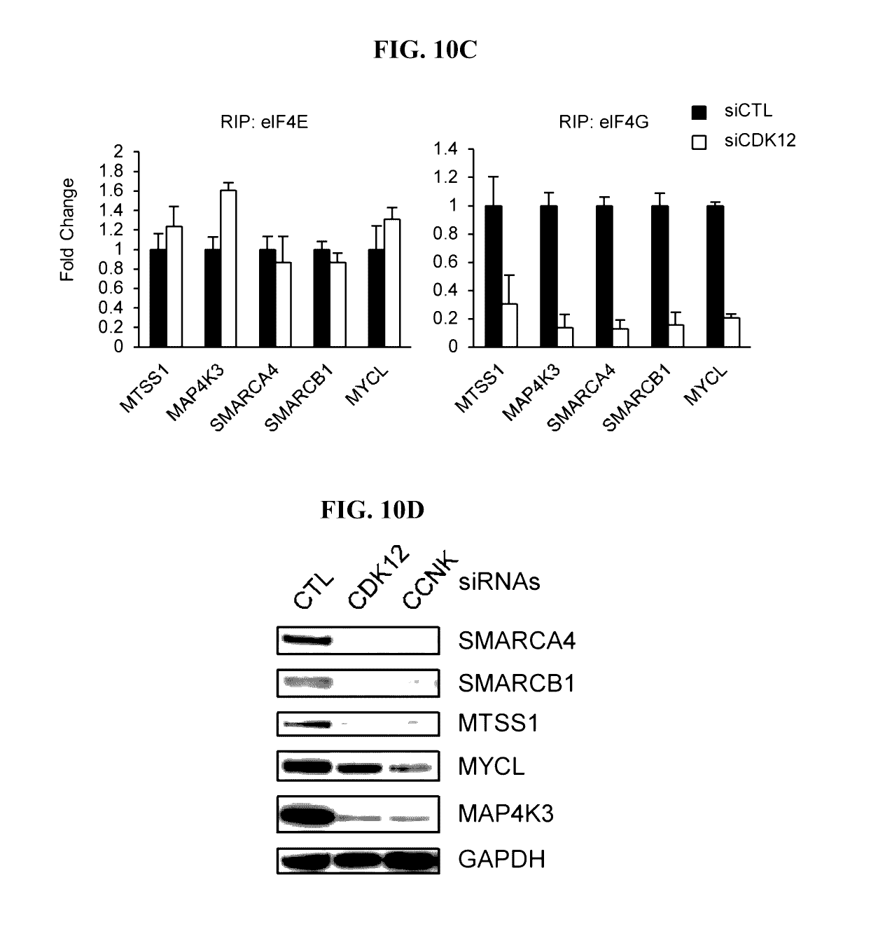

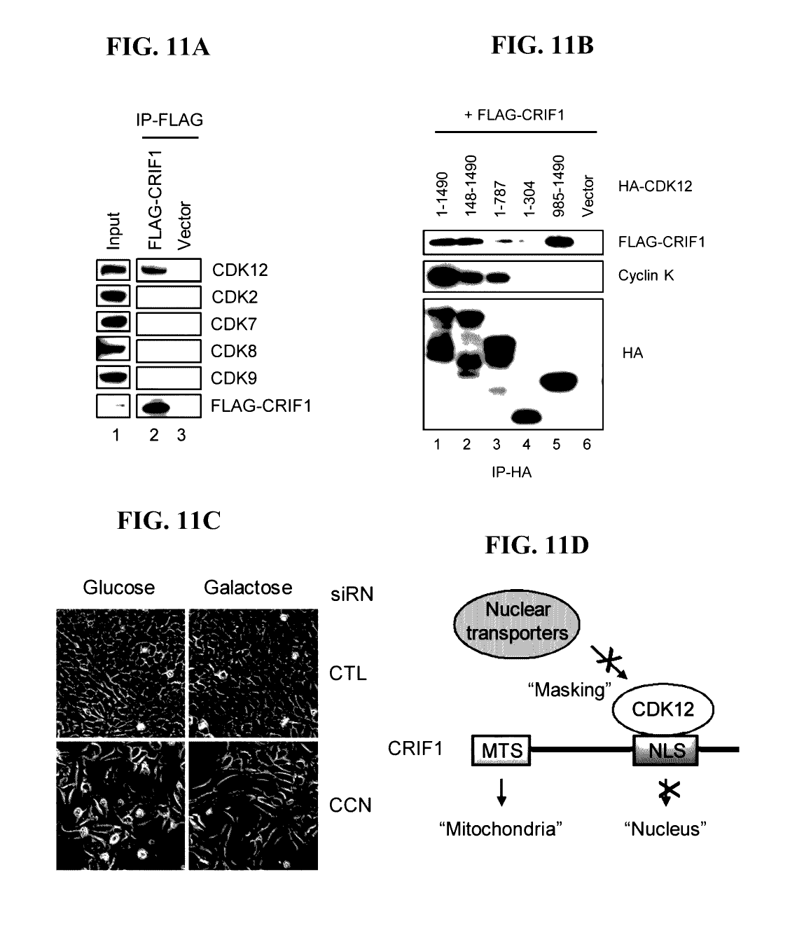

To further evaluate the relationship between CRIF1 and CDK12, the effects of CRIF1 depletion on CDK12 substrate phosphorylation was assessed. Knockdown of CRIF1 strongly increased global RNAPII-CTD Ser2P, without affecting RNAPII-CTD Ser5P levels (FIG. 6A). Moreover, depletion of CRIF1 increased CHK1 protein levels and S65, T70 phosphorylation of 4E-BP1 (FIGS. 6B and 6D respectively), without affecting transcription at the CHK1 gene (FIG. 6C) or CDK12:CCNK levels (FIG. 6D). These data indicate that CRIF1 antagonizes the effects of CDK12 on translation.

To assess whether CRIF1 can directly inhibit CDK12 kinase activity, in vitro kinase experiments were performed using purified recombinant CRIF1 (rCRIF1) protein and the human RNAPII GST-CTD (52 repeat) proteins as substrates, and the affinity-purified CDK12 kinase complex. As shown in FIG. 6E, recombinant CRIF1 potently inhibited CDK12-mediated phosphorylation of the human GST-CTD substrate in vitro, in a dose-dependent manner. Inhibition of GST-CTD phosphorylation increased the migration of the substrate in the SDS-PAGE, indicating that it affects the bulk of the GST-CTD substrate in the reaction. In contrast, rCRIF had no effect on the ability of the affinity-purified CDK9/P-TEFb complex to phosphorylate the GST-CTD substrate in vitro (FIG. 6F), indicating that inhibition by CRIF is highly selective for the CDK12.

It was determined whether CRIF1 also inhibits CDK12 phosphorylation of purified recombinant 4E-BP1 in vitro. To enable priming of 4E-BP1 by mTORC1, these reactions contained both affinity-purified CDK12 and the mTORC1/Raptor kinase complexes. As shown in FIG. 6G, rCRIF1 potently and selectively inhibited S65 and T70 phosphorylation of 4E-BP1 in vitro, in a dose-dependent manner, without affecting phosphorylation of the T37/46 sites by mTORC1. Consistent with a gain-of-function for CDK12 kinase activity in CRIF1 knockdown cells, enhanced binding of eIF4G to target mRNAs was observed (FIG. 6H, upper panel) that were identified previously by RIP-seq, with no effects on total mRNA levels (FIG. 6H, bottom panel). Thus, CRIF1 selectively inhibits CDK12-directed phosphorylation of the RNAPII CTD and 4E-BP1 substrates in vitro, and blocks translation of CDK12-dependent mRNAs in vivo.

### Example 10

### Oxidative Stress Disrupts Binding of CRIF1 to CDK12:CCNK

It was determined whether the effects of CDK12 and CRIF1 on DNA repair influence global poly-ADP-ribosylation (PARylation). CDK12 depletion disrupts double strand break (DSB) DNA repair and increases PARP activity, resulting in increased global PARylation and auto-PARylation of PARP1, as well as a reduction of CHK1 protein levels (FIG. 7A), and sensitizes cancer cells to PARP inhibitors (Joshi et al., J Biol Chem. 289(13):9247-53, 2014). PARP activity also increased in cells depleted of CHK1, consistent with its role in PARP-mediated single-stranded break repair, indicating that it contributes to this effect (FIG. 7B). In contrast, CIRF1 depletion decreased global PARylation levels (FIG. 7C), consistent with its ability to inhibit CDK12 activity, suggesting that reduced levels of CDK12 up-regulate PARP1 activity. Thus CDK12 may affect double strand break (DBS) repair via BRCA1 and other DNA response factors as well as PARP-mediated single strand break (SSB) repair via CHK1 (FIG. 7D, left panel). Consistent with this model, depletion of CDK12 sensitizes U2OS cells to the PARP inhibitor, Olaparib, in cell survival experiments (FIG. 7D, right panel).

PARP1 activity is also up-regulated in cells exposed to oxidative stress, accompanied by DNA-binding of the stress-responsive transcriptional activator, NRF2, and up-regulation of NRF2-dependent target genes (Wu et al., Free Radic Biol Med. 2014 February; 67:69-80, 2014). CDK12 was also recently shown to be essential for NRF2-dependent transcription (Li et al., Sci Rep 6, 21455, 2016). To examine whether CDK12 activity is regulated globally by oxidative stress in human cells, U2OS cells were treated with tBHQ (tert-butylhydroquinone) to induce mitochondrial oxidative stress. Under these conditions, a global increase in RNAPII CTD-Ser2 and 4E-BP1 S65, T70 phosphorylation was observed (FIG. 7F), consistent with increased CDK12 kinase activity. In contrast, the mTORC1-dependent 4E-BP1 T37/46 phosphorylation levels were high and only modestly increased in tBHQ-treated cells. Reciprocal co-immunoprecipitation experiments revealed that the association of CRIF1 with the CDK12:CCNK complex was greatly reduced in tBHQ-treated cells (FIG. 7E). In contrast, oxidative stress had no effect on the interaction of CCNK with CDK12. These data indicate that the interaction between CRIF1 and CDK12 is redox-sensitive, and that mitochondrial oxidative stress induces the selective release of CRIF1 from the CDK12 complex, which upregulates NRF2 pathways required for cell stress survival.

Thus, the CDK12:CCNK Ser/Thr-Pro kinase, which controls global levels of RNAPII CTD-Ser2 phosphorylation, also has an essential mRNA-specific role in translation and is regulated in a stress-specific manner by CRIF1, a mitoribosome-associated translation factor essential for mitochondrial oxidative phosphorylation. Previous studies have shown that human CDK12 controls transcription, mRNA processing, and 3′-end polyadenylation-termination through its effects on RNAPII CTD-Ser2 phosphorylation at a small number of genes. Notable targets include many DNA damage response genes, including BRCA1, 2, FANC1, FANCD2, and ATR. Consistent with these findings, inhibition or loss of CDK12 dramatically enhances cell killing when cancer cells are exposed to genotoxic stress, such as doxorubicin. Moreover, inhibition of CDK12 enables PARP1 inhibitors to inhibit the growth of cancer cells that expressing wild-type BRCA1, whereas normally these drugs act preferentially on BRCA1-mutant cells. The observations shown herein demonstrate that CDK12 also cooperates with mTORC1 to enhance translation in an mRNA-selective manner, and is upregulated in stress conditions through binding to a redox-sensitive inhibitor that controls respiratory growth, providing a new link between RNAPII phosphorylation and metabolic networks that affect translation in normal and stressed environments.

CDK12 Cooperates with mTORC1 to Phosophorylate 4E-BP1 and Regulate mRNA-Specific Translation:

The analysis of the role of CDK12 in expression of the CHK1 kinase and stabilization of p53 uncovered its unexpected role in translation. Several observations suggested the mRNA-selective role of CDK12 in translation. First, knockdown of CDK12 had a profound effect on de novo CHK1 protein biosynthesis and loading onto polysomes, without affecting mRNA levels or 3′end polyadenylation. Second, CDK12 was necessary for eIF4G to associate with CHK1 mRNA in RIP experiments. Furthermore, CDK12 directly phosphorylates the translation repressor, 4E-BP1, at S65 and T70, both in vivo and in vitro. Phosphorylation of 4E-BP1 by mTORC1 at T37, T46 has been shown to potentiate subsequent phosphorylation at S65 and T70, and consistent with these findings, affinity-purified mTORC1/Raptor strongly stimulated CDK12 phosphorylation at S65, T70 sites in vitro. Moreover, CDK12 phosphorylation of 4E-BP1 in vitro is diminished by point mutation of the mTORC1 phosphorylation sites. Lastly, knockdown of CDK12 largely eliminated phosphorylation of bulk 4E-BP1 (S65, T70), without affecting mTORC1 (T37, T46) phosphorylation in vivo.

RNA-immunoprecipitation in combination with high-throughput sequencing (RIP-seq) in U2OS cells revealed nearly 1000 mRNAs, most notably those involved in translation control and DNA damage response, that rely on CDK12 to release 4E-BP1 and associate with eIF4G. A significant subset of these are also sensitive to Rapamycin, an inhibitor of mTORC1. Other genes that bind eIF4G independently of mTORC1 or CDK12 were also identified, and presumably 4E-BP1 is phosphorylated by different kinases at these genes. Nearly half of the top fifty most sensitive CDK12-dependent genes were also strongly inhibited by Rapamycin, indicating that CDK12 and mTORC1 coordinately regulate translation of these mRNAs. ChIP data further indicate that 4E-BP1 associates with nascent RNA at the CHK1 promoter and other CDK12-regulated genes. Moreover, levels of 4E-BP1 and T70P-4E-BP1 at these genes are regulated by CDK12, indicating that 4E-BP1 bound to eIF4E at the mRNA cap may be phosphorylated and released cotranscriptionally, to enable these mRNAs to bind eIF4G for translation in the cytoplasm. The yeast homolog of CDK12, CTDK-I was also previously reported to affect translation, with effects on both initiation and elongation. Without being bound be theory, translation regulation may depend on targeting of CDK12, RNAPII CTD-Ser2P, or cap-binding translation factors to the nascent mRNAs at target genes. CDK12 also contains tandem RS domains that may interact directly with RNA or factors bound to the cap of nascent transcripts.

Selective Inhibition of CDK12 by the CRIF1 Mitochondrial OXPHOS Regulator:

Activation of CDK12 serves to protect cells from damage by genotoxic, thermal or oxidative stress, and increases transcription or translation of many DNA response genes. In yeast, CTDK-I/CDK12 is required for RNAPII CTD-Ser2P during the diauxic shift, when cells deplete the available glucose supply and shift from fermentation growth to oxygen-driven respiration. The kinase activity of CTDK-I is up-regulated by nutrient depletion and required for switching between nutrient growth conditions. In Drosophila, CDK12 is critical for transcription of oxidative stress-induced genes, including many antioxidants and oxidoreductants, and directly or indirectly blocks the expression of metabolic genes involved in amino acid, lipid and carbohydrate biosynthesis. Human CDK12 is required for induction of c-Fos transcription in EGFR signaling cells, and for self-renewal of human embryonic stem cells.

Thus it was demonstrated that CRIF1, a mitoribosome-associated translation factor required for expression of key mitochondrial oxidative phosphorylation proteins, is a highly selective inhibitor of CDK12. CDK12 binds to a regulatory loop near the CDK12 kinase active site, which has been shown previously to be required for kinase activity. This domain contains Cys residues, which are the target of small molecule inhibitors that can selectively block CDK12 activity. It was found that CRIF1 is displaced from CDK12 by oxidative stress, which upregulates CDK12 activity and its transcription and translation gene targets. Consistent with these findings, CDK12 is a coregulator of NRF2-induced genes, and that the NRF2 transcription factor additionally has an important role in translation. Moreover, CRIF1 inhibitsNRF2 stability, independently of KEAP1. Without being bound by theory, upon oxidative stress the loss of CRIF1 from the CDK12 complex both upregulates CDK12 kinase activity and stabilizes NRF2, leading the upregulation of antioxidant and DNA repair genes required for cells to survive stress.

CRIF1 itself is a top target gene for translation control by CDK12 and mTORC1. Thus CDK12 and mTORC1 control CRIF1 levels through a feedback loop, and analysis of GO categories indicates that translation control factors are a common target of this pathway. Without being bound by theory, CDK12 and CRIF constitute an important connection between the nucleus and mitochondrial for the regulation of networks that sense and respond to stress.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

1-4. (canceled)

5. An isolated nucleic acid molecule encoding a CRIF1 polypeptide comprising:
a) the amino acid sequence of SEQ ID NO: 2; or
b) the amino acid sequence of SEQ ID NO: 3,
wherein the polypeptide is at most 52 amino acids in length, and
wherein the nucleic acid molecule is operably linked to a heterologous promoter.

6. A vector comprising the nucleic acid molecule of claim 5.

7. The vector of claim 6, wherein the vector is a viral vector.

8. The vector of claim 7, wherein the viral vector is a poxviral vector, an adenoviral vector, an adeno-associated viral vector, or a lentiviral vector.

9. A composition comprising an effective amount of the vector of claim 6, and a pharmaceutically acceptable carrier.

10. The composition of claim 9, further comprising a chemotherapeutic agent.

11. The composition of claim 10, wherein the chemotherapeutic agent affects base excision repair.

12. The composition of claim 10, wherein the chemotherapeutic agent is an mTOR inhibitor, a PARP inhibitor, a Cdk inhibitor, a CHK1 inhibitor, or combinations thereof.

13. The composition of claim 9, further comprising an immunotherapeutic agent.

14. The composition of claim 13, wherein the immunotherapeutic agent is a PD-1 antagonist or a PD-L1 antagonist.

15. The composition of claim 14, wherein the PD-1 antagonist or the PD-L1 antagonist is an antibody or a siRNA.

16. A method for treating a subject with cancer, comprising administering to the subject a therapeutically effective amount of the composition of claim 9, thereby treating the cancer in the subject.

17. The method of claim 16, wherein the cancer is breast cancer, and wherein cells of the cancer expresses wild-type BRCA1 and/or wild-type BRCA2.

18. The method of claim 16, wherein the cancer is a breast cancer or ovarian cancer.

19-20. (canceled)

21. The method of claim 16, further comprising administering a therapeutically effective amount of a chemotherapeutic agent, wherein the chemotherapeutic agent affects base excision repair.

22. The method of claim 21, wherein the chemotherapeutic agent is an mTOR inhibitor, a PARP inhibitor, a CDK12 inhibitor, a CHK1 inhibitor, or combinations thereof.

23. The method of claim 16, further comprising administering to the subject a therapeutically effective amount of an immunotherapeutic agent.

24. The method of claim 23, wherein the immunotherapeutic agent is a PD-1 antagonist or a PD-L1 antagonist.

25. The nucleic acid molecule of claim 5, further encoding a tat protein or a nuclear localization sequence.

26. The nucleic acid molecule of claim 25, wherein the nuclear localization sequence is a Rev-1 nuclear localization sequence.

27. The nucleic acid molecule of claim 25, wherein the polypeptide consists of SEQ ID NO: 3.

28. The nucleic acid molecule of claim 25, wherein the polypeptide consists of SEQ ID NO: 2.
